(19) United States
(12) Patent Application Publication
(10) Pub. No.: US 2016/0195554 A1
(43) Pub. Date: Jul. 7, 2016

(54) Factors

(71) Applicant: OXFORD BIOMEDICA (UK) LIMITED, Oxfordshire (GB)

(72) Inventors: Richard Harrop, Oxfordshire (GB); Peter Treasure, Oxfordshire (GB)

(21) Appl. No.: 14/818,968

(22) Filed: Aug. 5, 2015

Related U.S. Application Data
(63) Continuation of application No. 13/286,868, filed on Nov. 1, 2011, now abandoned.

(30) Foreign Application Priority Data
Nov. 2, 2010 (GB) 1018480.2

(51) Int. Cl. G01N 33/80; G01N 33/68

(52) U.S. Cl. CPC G01N 33/80; G01N 33/6893; G01N 2800/52

(57) ABSTRACT

A method for determining a prognosis for benefit for a cancer patient receiving immunotherapy treatment involving (a) measuring a level of haematocrit and haemoglobin in a sample from the cancer patient, and (b) comparing the level of haematocrit in the sample to a reference level of platelets and comparing the level of haemoglobin in the sample to a reference level of haemoglobin, wherein a lower level of haematocrit and higher level of haemoglobin in the sample correlates with increased benefit to the patient.

### CROSS-REFERENCE TO RELATED APPLICATIONS

This is the a continuation of U.S. patent application Ser. No. 13/286,868, filed Nov. 1, 2011, which claims the benefit of GB Patent Application No. 1018480.2, filed Nov. 2, 2010, incorporated herein by reference in its entirety.

### FIELD OF THE INVENTION

The present invention relates to a method of cancer therapy which employs a factor or a set of factors to predict whether a patient will benefit from treatment with an immunotherapeutic agent.

In particular, the method predicts the clinical benefit to a potential patient of an MVA vector expressing a human 5T4 gene, such as TroVax®. More particularly, the method relates to those patients with renal, colorectal or prostate cancer.

### BACKGROUND TO THE INVENTION

Tumour cells are notoriously poor immunogens despite the fact that many antigens that are over-expressed or unique to tumour cells (tumour-associated antigens) have been identified. The reasons for this apparent lack of immunogenicity may be that cancer antigens are generally not presented to the immune system in a micro-environment that favours the activation of immune cells which would lead to the killing of the tumour cells; indeed, many tumour associated antigens are “self-antigens” and as such are subject to active immune tolerance mechanisms. Although no single known mechanism can explain poor tumour immunogenicity in all experimental models studied, the molecular basis can be separated conceptually into distinct groupings: i) lack of expression of co-stimulatory molecules essential for effective immune induction, ii) production of immuno-inhibitory substances and iii) variability in the expression of antigen by tumours.

Much progress has been made in the identification of tumour-associated antigens (TAA) that are potentially useful in the development of recombinant anti-cancer vaccines. TAAs can be divided into three major categories: i) non-self viral antigens e.g. E6/E7 from human papilloma virus (HPV), ii) altered self-antigens e.g. MUC-1 and iii) non-mutated self-antigens e.g. 5T4 and carcinoembryonic antigen (CEA).

Vaccinia virus (VV), a member of the poxvirus family, has been developed as a recombinant expression vector for the genetic delivery of antigens. Animals injected with a recombinant VV (rVV) have been shown to produce both antibody and CTL responses to the exogenous proteins. In contrast to tumour cells VV infection appears to create an optimal environment for the induction of an efficacious immune response. Recombinant VV expressing murine homologues of TAA, which, in murine models, are classed as self-antigens, have also been shown to induce TAA specific immune responses in murine models, illustrating that such constructs are potentially able to overcome immune tolerance to self-antigens. In vivo models demonstrate that the immune responses generated are able to prevent tumour establishment and in some cases are able to actively treat established tumours. These data also indicate that it is possible to turn an anti-viral response into an anti-cancer response by presenting a TAA in the context of viral antigens.

Recombinant VV vectors expressing the self-antigen CEA have been constructed and have been evaluated for toxicity and to a lesser extent efficacy in late stage colorectal cancer. Such rVV vectors were well tolerated and both antibody and cell mediated immune responses to the self-antigen CEA were reported. Lack of tumour response data in these trials may be due to the patient population which had very advanced tumours and had already failed prior chemotherapy. To date many people have been vaccinated with rVV and other poxviruses expressing TAAs in numerous cancer immunotherapy clinical trials. There have been no reports of toxicity either from the virus itself or as a result of the immune response induced to the TAA beyond local injection site reactions and transient pyrexia.

Suitable methods and suitable clinical markers, however, that can guide such immunotherapeutic methods would be extremely beneficial.

Renal cell carcinoma (RCC) has been reported to be the tenth most common cancer in the US and studies suggest a continued rise in RCC incidence. Although most patients with early stage RCC can be cured surgically, approximately 33% of patients present with metastatic disease for which the treatment is usually not curative. In addition, approximately 50% of patients who undergo potentially curative surgery for less advanced disease can be expected to develop a recurrence with distant metastases. Five-year survival for patients with de novo metastatic or recurrent disease ranges between 0% and 20%.

Clinical factors associated with prognosis of patients with metastatic RCC when they are treated with cytokines (interferon, and interleukin), chemotherapy or a variety of historic therapies have been reported to include tumour-, patient-, and disease-related factors, such as performance status (PS), time from diagnosis to therapy, number of metastatic sites, visceral metastasis, haemoglobin, calcium, lactate dehydrogenase, inflammation markers, and others.

Choueriri et al, Cancer (2007), 110(3): 543-550 reviewed the records of patients with metastatic renal cell carcinoma (RCC) who were treated with anti-VEGF agents—bevacizumab, sunitinib, sorafenib and axitinib—with a view to identifying patients who are more likely to benefit from these agents. The article reports that although many factors were associated individually with progression-free survival (PFS) on univariate analysis, only 5 factors were identified as independent predictors of a poor outcome on subsequent multivariate analysis. With the least favourable feature listed first, the following factors were identified: initial Eastern Cooperative Oncology Group performance status (ECOG PS)≧1 vs 0, time from diagnosis to current treatment <2 years vs years, abnormal baseline corrected serum calcium <8.5 mg/dL or >10 mg/dL vs 8.5-10 mg/dL, high platelet count >300 K/μL vs≦300 K/μL, and higher absolute neutrophil count (ANC) >4.5 K/μL vs ≦4.5 K/μL.

Choueriri et al however only teaches that these factors were associated with PFS for patients with metastatic RCC who received four specific VEGF-targeted therapies. It does not teach a skilled worker what factors may or may not be important for other therapies and other cancers. It is unclear whether the same factors reported previously are relevant to patients who are treated with, for example, immunotherapies.

Colorectal carcinoma (CRC) is one of the most common cancers in Western societies, being second only to lung cancer as a cause of death from malignancy. It is the second most common cancer in England and Wales. Approximately 24,000 men and women develop the disease each year, and over half of these die from it.

Fusek et al, World J Gastroenterol (2004), 10(13): 1890-1892 aimed to examine the calcium metabolism in patients with CRC and control patients. Seventy newly diagnosed CRC patients were included. The healthy control group was age and gender matched. They conclude that their results further strengthen the possibility that serum calcium might be a pathogenic and prognostic factor in the development of CRC. They say that their data draw attention to the possibility that by increasing calcium intake, the multi-levelled pathogenic process leading to tumourigenesis might be influenced. They go on to state that in order to prove this, further studies are necessary.

Fusek et al, however, does not indicate whether or not serum calcium might be a pathogenic and prognostic factor for any of the drug therapies used to treat CRC and other cancers.

Thus, there remains a need for suitable methods and suitable clinical markers that can guide immunotherapeutic methods.

### SUMMARY

The invention provides materials and methods that address one or more needs in the fields of cancer therapy, immunotherapy, or related fields.

Some aspects of the invention relate to materials and methods for identifying patients likely to benefit from an immunotherapy.

### STATEMENTS OF THE INVENTION

We have identified a number of pre-treatment factors which correlate with both antibody response to the immunotherapy and treatment benefit. The invention thus has important implications for the selection of patients for treatment. In particular we have found that in combination baseline haemoglobin and haematocrit levels, or other baseline factors associated with anaemia of chronic disease, optionally also in combination with baseline levels of antibody to a tumour associated antigen, are a significant predictor of treatment benefit.

Thus in a first aspect of the present invention there is provided a method for determining a prognosis for benefit for a cancer patient receiving immunotherapy treatment involving (a) measuring a level of haemoglobin and haematocrit in a sample from the cancer patient, and (b) comparing the levels of haemoglobin and haematocrit in the sample to respective reference levels of haemoglobin and haematocrit, wherein in combination a higher level of haemoglobin and a lower level of haematocrit in the sample correlates with increased benefit to the patient from immunotherapy treatment.

In a second aspect of the present invention there is provided a method for determining a prognosis for benefit for a cancer patient receiving immunotherapy treatment involving (a) measuring a level of mean corpuscular haemoglobin concentration (MCHC) in a sample from the cancer patient, and (b) comparing the level of MCHC in the sample to a reference level of MCHC, wherein a higher level of MCHC in the sample correlates with increased benefit to the patient from immunotherapy treatment.

In one embodiment the method according to and the first and/or second aspects involves additionally (c) measuring a level of red blood cell number in a sample from a cancer patient, and (d) comparing the level of red blood cell number in a sample to a reference level of red blood cell number, wherein a high level of red blood cell number correlates with increased benefit to the patient from immunotherapy treatment

According to a third aspect of the present invention there is provided a method for determining a prognosis for benefit for a cancer patient receiving immunotherapy treatment involving (a) measuring a level of mean corpuscular volume (MCV) in a sample from the cancer patient, and (b) comparing the level of MCV in the sample to a reference level of MCV, wherein a higher level of MCV in the sample correlates with increased benefit to the patient from immunotherapy treatment.

According to a fourth aspect of the present invention there is provided a method for determining a prognosis for benefit for a cancer patient receiving immunotherapy treatment involving (a) measuring a level of mean cell haemoglobin (MCH) in a sample from the cancer patient, and (b) comparing the level of MCH in the sample to a reference level of MCH, wherein a higher level of MCH in the sample correlates with increased benefit to the patient from immunotherapy treatment.

In one embodiment the method of the present invention involves additionally (c) or (d) measuring a level of total calcium in a sample from the cancer patient, and (d) or (e) comparing the level of total calcium in the sample to a reference level of total calcium, wherein a lower level of total calcium in the sample correlates with increased benefit to the patient from immunotherapy treatment.

In one embodiment the method of the present invention involves additionally (c) or (d) measuring a level of aspartamine transaminase (ASAT) in a sample from the cancer patient, and (d) or (e) comparing the level of ASAT in the sample to a reference level of ASAT, wherein a lower level of ASAT in the sample correlates with increased benefit to the patient from immunotherapy treatment.

In one embodiment the method of the present invention involves additionally (c) or (d) measuring a level of chloride in a sample from the cancer patient, and (d) or (e) comparing the level of chloride in the sample to a reference level of chloride, wherein a higher level of chloride in the sample correlates with increased benefit to the patient from immunotherapy treatment.

In one embodiment the method of the present invention involves additionally (c) or (d) measuring a level of sodium in a sample from the cancer patient, and (d) or (e) comparing the level of sodium in the sample to a reference level of sodium, wherein a higher level of sodium in the sample correlates with increased benefit to the patient from immunotherapy treatment.

In one embodiment the method of the present invention involves additionally (c) or (d) measuring a level of alanine transaminase (ALAT) in a sample from the cancer patient, and (d) or (e) comparing the level of ALAT in the sample to a reference level of ALAT, wherein a lower level of ALAT in the sample correlates with increased benefit to the patient from immunotherapy treatment.

In one embodiment the method of the present invention involves additionally (c) or (d) measuring a baseline level of an antibody to a tumor associated antigen in a sample from the cancer patient, and (d) or (e) comparing the baseline level of the an antibody to a tumor associated antigen in the sample to a reference level of antibody to a tumor associated antigen, wherein a lower baseline level of antibody to a tumor associated antigen in the sample correlates with increased benefit to the patient from immunotherapy treatment.

In one embodiment the method of the present invention additionally involves (c) or (d) measuring a level of at least one factor selected from the group consisting of: iron, ferritin, transferrin saturation, soluble transferrin receptor, total iron binding capacity, transferrin, zinc protoporphyrin, reticulocyte haemoglobin, bone marrow iron, hepcidin, C-reactive protein, interleukin 6, interleukin 10, vascular endothelial growth factor, interleukin 1, tumour necrosis factor alpha, and interferon gamma in a sample from the cancer patient receiving immunotherapy treatment, and (d) or (e) comparing the levels of said at least one factor to respective reference levels, wherein a higher level of iron, transferrin saturation, reticulocyte haemoglobin, or bone marrow iron or a lower level of ferritin, soluble transferrin receptor, zinc protoporphyrin, hepcidin, C-reactive protein, interleukin 6, interleukin 10, vascular endothelial growth factor, interleukin 1, tumour necrosis factor alpha, or interferon gamma or normal levels of total iron binding capacity (262-474 μg/dL) or transferrin (204-360 mg/dL) in the sample correlates with increased benefit to the patient from immunotherapy treatment.

According to a fifth aspect of the present invention there is provided a method for determining a prognosis for benefit for a cancer patient receiving immunotherapy treatment involving measuring the following factor in a sample from the cancer patient:

$$Factor = - 0.292 \times ( natural log of baseline level of an antibody to a tumor associated antigen ) + 0.0224 \times ( haemoglobin level in g \text{/} L ) + - 6.16 \times ( haematocrit as a fraction )$$

wherein a higher factor correlates with increased benefit to the patient from immunotherapy treatment.

According to a sixth aspect of the present invention there is provided a method for determining a prognosis for benefit for a cancer patient receiving immunotherapy treatment involving (a) measuring the factor of the present invention in a sample from the cancer patient, and (b) classifying the patient as belonging to either a first or second group of patients, wherein the first group of patients having a higher level of the factor is classified as having an increased likelihood of benefit than the second group of patients having a lower level of the factor.

In one embodiment the method of the present invention is for determining a prognosis for benefit for a cancer patient prior to receiving immunotherapy.

Although it is the combination of the levels, factors and/or measurements mentioned both above and below in relation to other aspects and embodiments of the invention that is useful in determining benefit, these do not need to be derived at the same time, i.e. whilst it may sometimes be convenient to carry out the method using the levels, factors and/or measurements from a single sample, this will not always be convenient. Thus, in one embodiment the method of the present invention a measurement or measurements is taken from one or more samples.

According to a seventh aspect of the present invention there is provided a method of predicting the responsiveness of a patient or patient population with cancer to treatment with immunotherapy, or for selecting patients or patient populations that will respond to immunotherapy, comprising comparing the differential levels of the factors as defined in any preceding aspect.

In one embodiment the tumor associated antibody useful in the present invention is selected from the group consisting of: 5T4, WT1, MUC1, LMP2, HPV E6 E7, EGFR vIII, Her-2/neu, Idiotype, MAGE A3, p53, NY-ESO-1, PSMA, GD2, CEA, MelanA.MART1, Ras mutant, gp100, Proteinase 3, bcr-abl, Tyrosinase, Survivin, PSA, hTERT, EphA2, PAP, ML-IAP, AFP, EpCAM, ERG (TMPRSS2 ETS) fusion, NA17, PAX3, ALK, Androgen receptor, Cyclin B1, Polysialic acid, MYCN, RhoC, TRP-2, GD3, Fucosyl GM1, Mesothelin, PSCA, MAGE A1, sLE, CYP1B1, PLAC1, GM3, BORIS, Tn, GloboH, ETV6-AML, NY-BR-1, RGS5, SART3, STn, Carbonic Anhydrase IX, PAX5, OY-TES1, Sperm protein 17, LCK, HMWMAA, AKAP-4, SSX2, XAGE1, B7H3, Legumain, Tie2, Page4, VEGFR2, MAD-CT-1, FAP, PDGFR-β, MAD-CT-2, Fos-related antigen 1.

In one embodiment the cancer for which benefit is predicted is bladder, bone, brain, breast, cervix, colorectal, eye, gallbladder, Hodgkin's lymphoma, Kaposi's sarcoma, kidney, larynx, leukemia, liver, lung, melanoma, mesothelioma, multiple myeloma, nasopharyngeal, non-Hodgkin lymphoma, oesophagous, oral, ovary, pancreas, penis, prostate, salivary gland, small intestine, stomach, testis, thyroid, uterus, vagina or vulva. More particularly the cancer is renal, prostate, breast, ovarian, colorectal cancer or mesothelioma.

In one embodiment the immunotherapy comprises use of a poxvirus vector.

In one embodiment the immunotherapy comprises use of 5T4 tumour associated antigen.

In methods involving the baseline level of antibody to a tumour, the associated antigen may be the baseline level of 5T4 antibody.

In one embodiment the immunotherapy comprises use of a Modified Vaccinia Ankara viral vector expressing the 5T4 tumour associated antigen.

In one embodiment the immunotherapy treatment comprises the use of a Modified Vaccinia Ankara viral vector expressing the human 5T4 tumour associated antigen gene under the regulatory control of a modified mH5 promoter.

Various features and embodiments of the invention will now be described by way of example.

### Measurement of Antibody Response

For ease of reference the measurement and determination of reference levels or baseline levels of an antibody to a tumour associated antigen will be discussed below by way of example only with reference to the method as carried out in the Examples and with reference to a Modified Vaccinia Ankara viral vector expressing (MVA) expressing the 5T4 tumour associated antigen; however it will be readily appreciated that similar methods can be used in relation to other tumour associated antigen antibody levels. Thus, in one embodiment, 5T4 and MVA-specific antibody responses were determined using a validated semi-quantitative ELISA. Polyclonal plasma, known to be positive for both 5T4 and MVA antibodies were used as a standard curve for each assay. The standard curves for each ELISA were assigned a nominal value of 5T4 or MVA antibody relative units (RU) and were titrated from 200 to 1.56 RU. A cut-point was established for each assay by analyzing 5T4 and MVA-specific antibody levels in plasma recovered from 50 healthy donors. Cut-points of 12.77RU and 5.20RU were established for 5T4 and MVA respectively by setting the false positive rate to be 5%. Variation in the level of 5T4 and MVA antibody levels was assessed in cancer patients who had not received any 5T4 or MVA targeted therapies. A 1.54 fold increase in 5T4 antibody and a 1.76 fold increase in MVA antibody was established as the level at which a 1% false positive rate could be expected.

All plasma test samples were analyzed, in a blinded manner, at a dilution of 1:50 for 5T4 or 1:2000 for MVA and results reported as relative units (RU) of 5T4 or MVA-specific antibodies. A positive response at baseline was reported if the pre-treatment antibody levels exceeded the cut-point. If necessary further determinations during immunotherapy can be conducted in which case a positive response following vaccination was reported if the antibody levels exceeded the cut-point and the increase, relative to the baseline, exceeded the pre-determined fold increase for each antigen (1.54 fold for 5T4 and 1.76 fold for MVA). In the Examples described below, samples were un-blinded once all analyses had been completed and the study had finished.

We have also developed a surrogate factor as a prognostic indicator for survival of patients who are receiving immunotherapy.

The present invention is in one embodiment based on a combination of baseline levels of haematocrit, haemaglobin and antibody which give rise to a surrogate for immune response. In more detail the surrogate factor was constructed as a linear combination of pre-treatment haemoglobin, haematocrit and antibody levels and was shown to be a significant predictor of treatment benefit.

Thus the present invention relates to a surrogate factor with formula:

$$IRS = - 0.292 \times ( natural log of baseline antibody level ) + 0.0224 \times ( haemoglobin level in g \text{/} L ) + - 6.16 \times ( haematocrit as a fraction )$$

where for ease of reference this surrogate factor is referred to as the “immune response surrogate” or “IRS”.
In one preferred embodiment the present invention relates to a surrogate factor with formula:

$$IRS = - 0.29184633 \times ( natural log of baseline antibody level ) + 0.022403243 \times ( haemoglobin level in g \text{/} L ) + - 6.159908434 \times ( haematocrit as a fraction )$$

In general terms the IRS can be expressed as:

$$IRS = - 0.291 to - 0.292 \times ( natural log of baseline antibody level ) + 0.0223 to 0.0224 \times ( haemoglobin level in g \text{/} L ) + - 6.15 to - 6.16 \times ( haematocrit as a fraction )$$

It is noteworthy that the sign associated with haematocrit is negative in the IRS despite being positive when the model just contained haematocrit. The form of the IRS is indicating that, for a given level of haemoglobin, response is negatively associated with haematocrit.

More generally in a further aspect of the present invention there is provided a method for determining a prognosis for benefit for a cancer patient receiving immunotherapy treatment involving (a) measuring an IRS as defined above in a sample from the cancer patient, and (b) classifying the patient as belonging to either a first or second group of patients, wherein the first group of patients having a higher level of the IRS is classified as having an increased likelihood of benefit than the second group of patients having a lower level of the IRS.

By way of example, in a relevant potential patient population, a suitable reference level of IRS may be a patient group within the 3^{rd} or 4^{th} quartile, or a suitable reference level may be an IRS level which include greater than or equal to 20%, 25%, 30%, 35%, 40%, 45%, 50%, 55%, 60%, 65%, 70% or 75% of the potential patient population whose IRS is measured.

### Anaemia

Sufficiently low haemoglobin (HGB) or haematocrit (HCT) value is characteristic of anaemia. Anaemia is a decrease in number of red blood cells (RBCs) or less than the normal quantity of haemoglobin in the blood. Anaemia can be caused by various conditions, most importantly through iron-deficiency (iron deficiency anaemia (IDA)) or as a result of chronic disease and/or inflammation, such as in the case of cancer, (anaemia of chronic disease (ACD)). Thus, the present invention also provides that as well as baseline haemoglobin/haematocrit levels, baseline iron levels may be a predictor of which cancer patients perform better with immunotherapy. Alternatively, other measures of iron levels, such as ferritin, soluble transferrin receptor levels or the level of transferrin saturation may be used. For example, iron deficiency can be determined by tests which can measure a low serum ferritin, a low serum iron level, an elevated serum transferrin level, serum transferrin saturation levels, zinc protoporphyrin, reticulocyte haemoglobin, bone marrow iron and a high total iron binding capacity (TIBC). Serum ferritin is the most sensitive laboratory test for iron deficiency anaemia. Thus, although we refer to the use of iron baseline count as being useful as a prognostic indicator, the measure may equally well be expressed as ferritin, serum transferrin or another factor which is used to determine iron levels. In addition, factors associated with the anaemia of chronic disease may also predict which cancer patients perform better with immunotherapy. These factors include ferritin, hepcidin, C-reactive protein (CRP), interleukin 6 (IL-6), interleukin 10 (IL-10), vascular endothelial growth factor (VEGF), interleukin 1 (IL-1), tumour necrosis factor alpha (TNF-α), and interferon gamma (IFN-γ). For example, anaemia of chronic disease can be indicated by tests which can measure a normal or high serum ferritin, an elevated serum hepcidin, serum CRP, serum IL-1, serum IL-6, serum IL-10, serum TNF-α, serum IFN-γ, or serum VEGF. Thus, although we refer to anaemia of chronic disease as being useful as a prognostic indicator, the measure may equally well be expressed as ferritin, hepcidin or another factor which is used to determine anaemia of chronic disease.

Anaemia is often first shown by routine blood tests, which generally include a complete blood count (CBC), a high red blood cell distribution width (RDW), reflecting a varied size distribution of red blood cells (RBCs), a low MCV, MCH or MCHC and these or other related levels can also be used in the present invention. Iron deficiency anaemia can be discriminated from anaemia of chronic disease by assessment of serum ferritin levels, with below normal levels being associated with IDA and normal or elevated levels being associated with ACD.

Thus in a further aspect of the present invention there is provided a method for determining a prognosis for benefit for a cancer patient receiving immunotherapy treatment involving (a) measuring a baseline level of an antibody to a tumour associated antigen, and at least one factor selected from the group consisting of iron, ferritin, transferrin saturation, soluble transferrin receptor, total iron binding capacity, transferrin, zinc protoporphyrin, reticulocyte haemoglobin, bone marrow iron, hepcidin, C-reactive protein, interleukin 6, interleukin 10, vascular endothelial growth factor, interleukin 1, tumour necrosis factor alpha, and interferon gamma, mean corpuscular volume, mean corpuscular haemoglobin concentration, and red blood cells in a sample from the cancer patient immunotherapy treatment, and (b) comparing the levels of the tumour associated antigen antibody, and said at least one factor to respective reference levels, wherein a lower level of tumour associated antibody, and a higher level of iron, transferrin saturation, mean corpuscular volume, mean corpuscular haemoglobin concentration, red blood cells, reticulocyte haemoglobin, or bone marrow iron, or a lower level of ferritin, soluble transferrin receptor, zinc protoporphyrin, hepcidin, C-reactive protein, interleukin 6, interleukin 10, vascular endothelial growth factor, interleukin 1, tumour necrosis factor alpha, or interferon gamma or normal levels of total iron binding capacity (262-474 μg/dL) or transferrin (204-360 mg/dL) in the sample correlates with increased benefit to the patient from immunotherapy treatment.

The present invention thus involves classifying patients according to differential levels or levels in relation to commonly used reference levels. Examples of such levels are given below by way of example only. In general terms the interpretation of any clinical laboratory test involves comparing the patient's results with the test's reference range, which are commonly known or published. In general terms the first step in determining a reference range is to define the population to which the range will apply. A large number of individuals from a group who are thought to represent a “normal” population, will be tested for a particular laboratory test. The reference range is then derived mathematically by taking the average value for the group and allowing for natural variation around that value (plus or minus 2 standard deviations from the average). In this way, ranges quoted by labs will represent the values found in 95% of individuals in the chosen ‘reference’ group. In other words, even in a “normal” population, a test result will lie outside the reference range in 5% of cases (1 in 20). This is why the term “reference range” is preferred over “normal range”. Whether or not the test result is within the laboratory reference range, the result must be considered within the context of the patient's personal circumstances, and with the benefit of knowledge of the patient's past medical history, current medication and the results of any other investigations. However, the present invention has an advantage in that it utilises the results of commonly carried out tests. It will also be appreciated that measuring levels of these factors can be carried out by a skilled worker in any suitable laboratory.

As previously mention iron levels are generally detected indirectly, for example with reference to ferritin levels; however, the following reference levels may also be applied:
Total Serum Iron (TSI) 76-198 μg/dL (Male)
   26-170 μg/dL (Female)
Total Iron-Binding Capacity (TIBC) 262-474 μg/dL

### Haematocrit Levels

It has previously been reported that a higher haematocrit level was associated with increased immunotherapy benefit. However, surprisingly we have now identified that immunotherapy performs better in cancer patients with a lower baseline haematocrit level, in the context of haemoglobin level and optionally also baseline antibody levels.

By “lower level” we include patients who have a level of baseline haematocrit below the median for a patient in need of immunotherapy or below or towards the lower end of reference levels.

Thus the present invention provides a method for determining a prognosis for benefit for a cancer patient receiving immunotherapy treatment involves (a) measuring a level of haematocrit in a sample from the cancer patient, and (b) classifying the patient as belonging to either a first or second group of patients, wherein the first group of patients having low levels of haematocrit is classified as having an increased likelihood of benefit than the second group of patients having high levels of haematocrit when considered in the context of haemoglobin and baseline antibody levels.

By “receiving immunotherapy” here and elsewhere we particularly mean patients who are being assessed for immunotherapy, i.e. patients who could potentially benefit from treatment for cancer.

In other words the present invention provides a method of predicting the responsiveness of a patient or patient population with cancer to treatment with immunotherapy, or for selecting patients or patient populations that may respond to immunotherapy, which comprises comparing the differential levels of haematocrit wherein a haematocrit level below a reference level is associated with benefit, when considered in the context of haemoglobin and baseline antibody levels.

By “lower level” we include a patient or patient population who has a level of haematocrit either below a reference level for a patient or patient population who have been diagnosed with cancer and are therefore in need of treatment, such as immunotherapy, or below a reference level for a normal individual or population. By “reference level” we include a level which represents a level below which the administration of immunotherapy will confer a clinical benefit to the patient or patient population, such as improved overall survival, increased progression-free survival, or decreased risk of tumour recurrence or spread.

In one embodiment the lower level will include a patient or patient population who have a level of haematocrit below the median for a patient or patient population who have been diagnosed with cancer and are therefore in need of treatment, such as immunotherapy; it will also include a patient or patient population who have a level of haematocrit which is below the median for a normal individual or patient population.

In relation to haematocrit, the haematocrit levels in a normal patient population has been reported (given as a percentage of 1.0) as 0.410-0.500 (males 18-64 years), 0.360-0.490 (males 65+ years), 0.350-0.460 (females 18-64 years), and 0.330-0.460 (65+ years), and references to a high or higher amount can be determined accordingly and with reference to the afore-mentioned definitions.

As mentioned above if we just look at haematocrit in general we would like higher values of haematocrits. However, surprising lower values of haematocrit are good in the context of haemoglobin and optionally also baseline antibody levels. This means in practice that the relevant reference level for haematocrits will depend on the observed haemoglobin levels as will be appreciated by a skilled worker.

### Haemoglobin Levels

We have previously identified that immunotherapy performs better in cancer patients with a higher baseline haemoglobin level (see for example our WO 2010/079339). We have also found that there is an association between the level of haemoglobin and level of haematocrits and the magnitude of antibody response and enhanced clinical benefit.

Thus, in one aspect the invention provides a method for predicting the magnitude of an antibody response to an immunotherapy in a cancer patient receiving the immunotherapy treatment which involves (a) measuring a level of haemoglobin in a sample from the cancer patient, and (b) comparing the level of haemoglobin in the sample to a reference level of haemoglobin, wherein a higher level of haemoglobin in the sample correlates with increased benefit to the patient when considered in the context of haematocrit and optionally also baseline antibody levels.

By “higher level” we include patients who have a level of baseline haemoglobin at or above the median for a patient in need of immunotherapy or above or towards the upper end of normal levels.

By “higher level” we include a patient or patient population who has a level of haemoglobin at or above a reference level for a patient or patient population who have been diagnosed with cancer and are therefore in need of treatment, such as immunotherapy; or above a reference level for a normal individual or population. By “reference level” we include a level which represents a level at or above which the administration of immunotherapy will confer a clinical benefit to the patient or patient population, such as improved overall survival, increased progression-free survival, decreased risk of tumour recurrence or spread.

In one embodiment the higher level will include a patient or patient population who has a level of haemoglobin at or above the median for a patient or patient population who have been diagnosed with cancer and are therefore in need of treatment, such as immunotherapy; or at or above the median for a normal individual or patient population.

In one embodiment, the haemoglobin level associated with a more favourable outcome is about ≧100 g/L, or more preferably ≧120 g/L, even more preferably about ≧125 g/L, about ≧130 g/L or about ≧132 g/L. In one even more preferred embodiment, the haemoglobin level associated with a more favourable outcome is greater than about ≧140 g/L or about ≧145 g/L. In an especially preferred embodiment, the haemoglobin level associated with a more favourable outcome is about ≧153 g/L. These levels may be particularly associated with patients with RCC or CRC.

The haemoglobin level in a normal population has been reported as being in the range of 118-168 g/L. Haemoglobin levels have however been reported to vary according to gender and age. Thus, the reference range of 118-168 g/L has been reported as being the normal range for males of 65+ years. For males of 18-64 years the haemoglobin level in a normal population has been reported as being in the range of 138-172 g/L. For females of 18-64 years the haemoglobin level in a normal population has been reported as being in the range of 120-156 g/L. For females of 65+ years the haemoglobin level in a normal population has been reported as being in the range of 111-155 g/L.

### Ferritin

Serum ferritin levels are routinely measured in patients as part of the iron studies. Ferritin levels measured have a direct correlation with the total amount of iron stored in the body including cases of anaemia of chronic disease.

We have identified that immunotherapy performs better in cancer patients with certain baseline factor levels as described herein. We have identified that this result also holds true for patients with at or around normal ferritin levels, including serum ferritin levels. We have also found that there is an association between the level of ferritin and the magnitude of antibody response and enhanced clinical benefit.

Thus, in one aspect the invention provides a method for predicting the magnitude of an antibody response to an immunotherapy in a cancer patient receiving the immunotherapy treatment which involves (a) measuring a level of ferritin in a sample from the cancer patient, and (b) comparing the level of ferritin in the sample to a reference level of ferritin, wherein a lower level of ferritin in the sample correlates with increased benefit to the patient.

By “lower level” we include patients who have a level of baseline ferritin at or below the median for a patient in need of immunotherapy or at or below the upper end of normal levels.

By “lower level” we include a patient or patient population who has a level of ferritin at or below a reference level for a patient or patient population who have been diagnosed with cancer and are therefore in need of treatment, such as immunotherapy; or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of ferritin at or below the median for a patient or patient population who have been diagnosed with cancer and are therefore in need of treatment, such as immunotherapy; or at or above the median for a normal individual or patient population.

In the setting of anaemia, serum ferritin is the most sensitive lab test for iron deficiency anaemia and one of the best discriminators between IDA and ACD, wherein ferritin levels are reduced in IDA and normal or increased in ACD.

In a normal range study with urine and serum/plasma samples from healthy donors the following ranges have been established with ferritin tests:

| | | |
|---|---|---|
| Female: 20-50 years | 22-112 | ng/ml |
| Female: 65-90 years | 13-651 | ng/ml |
| Male: 20-50 years | 34-310 | ng/ml |
| Male: 65-87 years | 4-665 | ng/ml |
| Umbilical cord blood: | 30-276 | ng/ml |
| Infants: 0.5 month | 90-628 | ng/ml |
| Infants: 1 month | 144-399 | ng/ml |
| Infants: 2 month | 87-430 | ng/ml |
| Infants: 4 month | 37-223 | ng/ml |
| Infants: 6 month | 19-142 | ng/ml |
| Infants: 9 month | 14-103 | ng/ml |
| Infants: 12 month | 1-99 | ng/ml |
| Children: 6 month- | 7-142 | ng/ml |
| 15 years | | |

However a reference or baseline ferritin blood level may determined by many testing laboratories, such as LabCorp, using the Roche enhanced chemiluminescence immunoassay (ECLIA) methodology. The Roche ECLIA reference ranges for ferritin are 30-400 ng/mL for males, and 13-150 ng/mL for females. Other tests are in usage that rely on different methods and may have different reference ranges. In very general terms low ferritin levels of <50 ng/mL have been associated with related symptoms.

### Transferrin and Total Iron Binding Capacity

Transferrin is a glycoprotein that binds iron very tightly but reversibly. Although iron bound to transferrin is less than 0.1% of the total body iron, it is the most important iron pool, with the highest rate of turnover. Total iron-binding capacity (TIBC) is a medical laboratory test that measures the blood's capacity to bind iron with transferrin. It is performed by drawing blood and measuring the maximum amount of iron that it can carry, which indirectly measures transferrin since transferrin is the most dynamic carrier. TIBC is less expensive than a direct measurement of transferrin. Transferrin can be measured using commercially available immunoassays. TIBC can be measured using commercially available colourimetric iron-binding assays.

We have identified that immunotherapy performs better in cancer patients with a certain baseline factor levels as described herein. We have identified that this result also holds true for patients with at or around normal transferrin and TIBC levels. Normal reference ranges for transferrin are between 204-360 mg/dL and for TIBC are 262-474 μg/dL. We have also found that there is an association between the level of transferrin and TIBC and the magnitude of antibody response and enhanced clinical benefit.

### Transferrin Saturation

Transferrin saturation, measured as a percentage, is a medical laboratory value. It is the ratio of serum iron and total iron-binding capacity, multiplied by 100.

In both anaemia of chronic disease and iron-deficiency anaemia, the serum concentration of iron and transferrin saturation are reduced, reflecting absolute iron deficiency in iron-deficiency anaemia and hypoferremia due to acquisition of iron by the reticuloendothelial system in anaemia of chronic disease. In the case of anaemia of chronic disease, the decrease in transferrin saturation is primarily a reflection of decreased levels of serum iron. In iron-deficiency anaemia, transferrin saturation may be even lower because serum concentrations of the iron transporter transferrin are increased, whereas transferrin levels remain normal or are decreased in anaemia of chronic disease.

We have identified that immunotherapy performs better in cancer patients with a certain baseline factor levels described herein. We have now identified that this result also holds true for patients with at or around normal transferrin saturation levels. We have also found that there is an association between the level of transferrin saturation and the magnitude of antibody response and enhanced clinical benefit.

Thus, in one aspect the invention provides a method for predicting the magnitude of an antibody response to an immunotherapy in a cancer patient receiving the immunotherapy treatment which involves (a) measuring a level of transferrin saturation in a sample from the cancer patient, and (b) comparing the level of transferrin saturation in the sample to a reference level of transferrin saturation, wherein a higher level of transferrin saturation in the sample correlates with increased benefit to the patient.

By “higher level” we include patients who have a level of baseline transferrin saturation at or above the median for a patient in need of immunotherapy or above or towards the upper end of normal levels.

By “higher level” we include a patient or patient population who has a level of transferrin saturation at or above a reference level for a patient or patient population who have been diagnosed with cancer and are therefore in need of treatment, such as immunotherapy; or above a reference level for a normal individual or population. By “reference level” we include a level which represents a level at or above which the administration of immunotherapy will confer a clinical benefit to the patient or patient population, such as improved overall survival, increased progression-free survival, decreased risk of tumour recurrence or spread.

In one embodiment the higher level will include a patient or patient population who has a level of transferrin saturation at or above the median for a patient or patient population who have been diagnosed with cancer and are therefore in need of treatment, such as immunotherapy; or at or above the median for a normal individual or patient population.

A normal transferrin saturation level is: 15-50% (males), 12-45% (females) μg/dl=micrograms per deciliter.

However, as mentioned above laboratories often use different units and “normal” may vary by population and the lab techniques used. A skilled worker would therefore consult the individual laboratory reference values to interpret a specific test.

Soluble Transferrin Receptor and/or Zinc Protoporphyrin

We have identified that immunotherapy performs better in cancer patients with a certain baseline factor levels described herein. We have now identified that this result holds true for patients with at or below normal transferrin receptor levels. We have also found that there is an association between the level of transferrin receptor and the magnitude of antibody response and enhanced clinical benefit. The same also holds true for zinc protoporphyrin.

Transferrin receptor (TfR) is a carrier protein for transferrin. It is needed for the import of iron into the cell and is regulated in response to intracellular iron concentration. Low iron concentrations promote increased levels of transferrin receptor, to increase iron intake into the cell. Thus, for example a high serum transferrin receptor level in anaemia has been found to indicate coexistent iron deficiency and/or anaemia of chronic disease.

Thus, in one aspect the invention provides a method for predicting the magnitude of an antibody response to an immunotherapy in a cancer patient receiving the immunotherapy treatment which involves (a) measuring a level of transferrin receptor and/or zinc protoporphyrin in a sample from the cancer patient, and (b) comparing the level of transferrin receptor in the sample to a reference level of transferrin receptor and/or zinc protoporphyrin respectively, wherein a lower level of transferrin receptor and/or zinc protoporphyrin in the sample correlates with increased benefit to the patient.

By “lower level” we include patients who have a level of baseline transferrin receptor and/or zinc protoporphyrin at or below the median for a patient in need of immunotherapy or below or towards the lower end of normal levels.

By “lower level” we include a patient or patient population who has a level of transferrin receptor and/or zinc protoporphyrin at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of transferrin receptor at or below the median for a patient or patient population who have been diagnosed with cancer and are therefore in need of treatment, such as immunotherapy; or at or below the median for a normal individual or patient population.

Serum transferrin receptor levels in normal patients have been reported for example as 0.9-3.0 μg/dL, and high levels in chronic iron deficiency anaemia as 4.2-19.2 μg/dL.

### Mean Corpuscular Haemoglobin Concentration

Since the haematocrit (Ht or HCT) or packed cell volume (PCV) or erythrocyte volume fraction (EVF) is the proportion of blood volume that is occupied by red blood cells it is considered an integral part of a person's complete blood count results, along with haemoglobin concentration and therefore represents a simple way of determining a patient's suitability for immunotherapy.

The mean corpuscular haemoglobin concentration, or MCHC, is a measure of the concentration of haemoglobin in a given volume of packed red blood cells (i.e. haemoglobin divided by haematocrit). Thus, in one aspect the invention provides a method for determining a prognosis for benefit for a cancer patient receiving immunotherapy treatment involving (a) measuring a level of MCHC in a sample from the cancer patient, and (b) comparing the level of MCHC in the sample to a reference level of MCHC, wherein a higher level of MCHC in the sample correlates with increased benefit to the patient.

By “higher level” we include patients who have a level of baseline MCHC at or above the median for a patient in need of immunotherapy.

A further method for determining a prognosis for benefit for a cancer patient receiving immunotherapy treatment involves (a) measuring a level of MCHC in a sample from the cancer patient, and (b) classifying the patient as belonging to either a first or second group of patients, wherein the first group of patients having high levels of MCHC is classified as having an increased likelihood of benefit than the second group of patients having low levels of MCHC.

The invention also provides a method for monitoring the effectiveness of a course of treatment for a patient with cancer. The method involves (a) determining a level of MCHC in a sample from the cancer patient prior to immunotherapy treatment, and (b) determining the level of MCHC in a sample from the patient after treatment, whereby comparison of the MCHC level prior to treatment with the MCHC level after treatment indicates the effectiveness of the treatment.

In other words the present invention provides a method of predicting the responsiveness of a patient or patient population with cancer to treatment with immunotherapy, or for selecting patients or patient populations that may respond to immunotherapy comprising comparing the differential levels of MCHC wherein a MCHC level at or above a reference level is associated with benefit.

By “higher level” we include a patient or patient population who has a level of MCHC either above a reference level for a patient or patient population who have been diagnosed with cancer and are therefore in need of treatment, such as immunotherapy; or above a reference level for a normal individual or population. By “reference level” we include a level which represents a level above which the administration of immunotherapy will confer a clinical benefit to the patient or patient population such as overall survival, increased progression-free survival decreased risk of tumour recurrence or spread.

In one embodiment the higher level will include a patient or patient population who have a level of MCHC at or above the median for a patient or patient population who have been diagnosed with cancer and are therefore in need of treatment, such as immunotherapy; or it will also include a patient or patient population who have a level of MCHC which is at or above the median for a normal individual or patient population.

MHCH is calculated by dividing the haemoglobin by the haematocrit. Reference ranges for blood tests are 32 to 36 g/dL, or between 4.9 to 5.5 mmol/L. It is thus a mass or molar concentration. Still, many instances measure MCHC in percentage (%), as if it was a mass fraction (m_{Hb}/m_{RBC}). Numerically, however, the MCHC in g/dL and the mass fraction of haemoglobin in red blood cells in % are identical, assuming a RBC density of 1 g/mL and negligible haemoglobin in plasma.

### Mean Corpuscular Volume

The mean corpuscular volume, or “mean cell volume” (MCV), is a measure of the average red blood cell volume that is reported as part of a standard complete blood count. In patients with anaemia, it is the MCV measurement that allows classification as either a microcytic anaemia (MCV below normal range), normocytic anaemia (MCV within normal range) or macrocytic anaemia (MCV above normal range).

It can be calculated (in litres) by dividing the haematocrit by the red blood cell count (number of red blood cells per litre). The result is typically reported in femtolitres.

If the MCV was determined by automated equipment, the result can be compared to RBC morphology on a peripheral blood smear. Any deviation would be indicative of either faulty equipment or technician error.

The reference range is typically 80-100 fL.

We report that immunotherapy performs better in cancer patients with a higher MCV. We have also found that there is an association between the level of MCV and the magnitude of antibody response and enhanced clinical benefit.

Thus, in one aspect the invention provides a method for determining a prognosis for benefit for a cancer patient receiving immunotherapy treatment involving (a) measuring a level of MCV in a sample from the cancer patient, and (b) comparing the level of MCV in the sample to a reference level of MCV, wherein a higher level of MCV in the sample correlates with increased benefit to the patient.

By “higher level” we include patients who have a level of baseline MCV at or above the median for a patient in need of immunotherapy.

A further method for determining a prognosis for benefit for a cancer patient receiving immunotherapy treatment involves (a) measuring a level of MCV in a sample from the cancer patient, and (b) classifying the patient as belonging to either a first or second group of patients, wherein the first group of patients having high levels of MCV is classified as having an increased likelihood of benefit than the second group of patients having low levels of MCV.

The invention also provides a method for monitoring the effectiveness of a course of treatment for a patient with cancer. The method involves (a) determining a level of MCV in a sample from the cancer patient prior to immunotherapy treatment, and (b) determining the level of MCV in a sample from the patient after treatment, whereby comparison of the MCV level prior to treatment with the MCV level after treatment indicates the effectiveness of the treatment.

In other words the present invention provides a method of predicting the responsiveness of a patient or patient population with cancer to treatment with immunotherapy, or for selecting patients or patient populations that may respond to immunotherapy comprising comparing the differential levels of MCV wherein a MCV level at or above a reference level is associated with benefit.

By “higher level” we include a patient or patient population who has a level of MCV either at or above a reference level for a patient or patient population who have been diagnosed with cancer and are therefore in need of treatment, such as immunotherapy; or at or above a reference level for a normal individual or population. By “reference level” we include a level which represents a level above which the administration of immunotherapy will confer a clinical benefit to the patient or patient population such as overall survival, increased progression-free survival decreased risk of tumour recurrence or spread.

In one embodiment the higher level will include a patient or patient population who have a level of MCV above the median for a patient or patient population who have been diagnosed with cancer and are therefore in need of treatment, such as immunotherapy; or it will also include a patient or patient population who have a level of MCV which is at or above the median for a normal individual or patient population.

In a further embodiment, it can be used to calculate red blood cell distribution width (RDW) and this measurement may also be used in the present invention.

### Mean Corpuscular Haemoglobin

The mean corpuscular haemoglobin, or “mean cell haemoglobin” (MCH), is the average mass of haemoglobin per red blood cell in a sample of blood. It is reported as part of a standard complete blood count. MCH value is diminished in hypochromic anaemias.

It is calculated by dividing the total mass of haemoglobin by the number of red blood cells in a volume of blood:

MCH=(Hgb)/RBC

A normal reference value in humans is 27 to 31 picograms/cell. Conversion to SI-units: 1 pg of haemoglobin=0,06207 femtomol. Normal value converted to SI-units: 1.68-1.92 fmol/cell.

We report that immunotherapy performs better in cancer patients with a higher MCH. We have also found that there is an association between the level of MCH and the magnitude of antibody response and enhanced clinical benefit.

Thus, in one aspect the invention provides a method for determining a prognosis for benefit for a cancer patient receiving immunotherapy treatment involving (a) measuring a level of MCH in a sample from the cancer patient, and (b) comparing the level of MCH in the sample to a reference level of MCH, wherein a higher level of MCH in the sample correlates with increased benefit to the patient.

By “higher level” we include patients who have a level of baseline MCH at or above the median for a patient in need of immunotherapy.

A further method for determining a prognosis for benefit for a cancer patient receiving immunotherapy treatment involves (a) measuring a level of MCH in a sample from the cancer patient, and (b) classifying the patient as belonging to either a first or second group of patients, wherein the first group of patients having high levels of MCH is classified as having an increased likelihood of benefit than the second group of patients having low levels of MCH.

The invention also provides a method for monitoring the effectiveness of a course of treatment for a patient with cancer. The method involves (a) determining a level of MCH in a sample from the cancer patient prior to immunotherapy treatment, and (b) determining the level of MCH in a sample from the patient after treatment, whereby comparison of the MCH level prior to treatment with the MCH level after treatment indicates the effectiveness of the treatment.

In other words the present invention provides a method of predicting the responsiveness of a patient or patient population with cancer to treatment with immunotherapy, or for selecting patients or patient populations that may respond to immunotherapy comprising comparing the differential levels of MCH wherein a MCH level above a reference level is associated with benefit.

By “higher level” we include a patient or patient population who has a level of MCH either above a reference level for a patient or patient population who have been diagnosed with cancer and are therefore in need of treatment, such as immunotherapy; at or above a reference level for a normal individual or population. By “reference level” we include a level which represents a level above which the administration of immunotherapy will confer a clinical benefit to the patient or patient population such as overall survival, increased progression-free survival decreased risk of tumour recurrence or spread.

In one embodiment the higher level will include a patient or patient population who have a level of MCH above the median for a patient or patient population who have been diagnosed with cancer and are therefore in need of treatment, such as immunotherapy; or it will also include a patient or patient population who have a level of MCH which is at or above the median for a normal individual or patient population.

### Red Blood Cells

As part of a complete blood cell count it is usual for the number of red blood cells to be determined. Too few red blood cells means a person has anaemia. A low number of red blood cells (RBCs) is usually seen with either a low haemoglobin or a low haematocrit level, or both.

Thus, in one aspect the invention provides a method for determining a prognosis for benefit for a cancer patient receiving immunotherapy treatment involving (a) measuring a level of RBCs in a sample from the cancer patient, and (b) comparing the level of RBCs in the sample to a reference level of RBCs, wherein a higher level of RBCs in the sample correlates with increased benefit to the patient. We have also found that there is an association between the level of RBCs and the magnitude of antibody response and enhanced clinical benefit.

By “higher level” we include patients who have a level of baseline RBCs at or above the median for a patient in need of immunotherapy.

A further method for determining a prognosis for benefit for a cancer patient receiving immunotherapy treatment involves (a) measuring a level of RBCs in a sample from the cancer patient, and (b) classifying the patient as belonging to either a first or second group of patients, wherein the first group of patients having high levels of RBCs is classified as having an increased likelihood of benefit than the second group of patients having low levels of RBCs.

The invention also provides a method for monitoring the effectiveness of a course of treatment for a patient with cancer. The method involves (a) determining a level of RBCs in a sample from the cancer patient prior to immunotherapy treatment, and (b) determining the level of RBCs in a sample from the patient after treatment, whereby comparison of the RBC level prior to treatment with the RBC level after treatment indicates the effectiveness of the treatment.

In other words the present invention provides a method of predicting the responsiveness of a patient or patient population with cancer to treatment with immunotherapy, or for selecting patients or patient populations that may respond to immunotherapy comprising comparing the differential levels of RBCs wherein a RBC level at or above a reference level is associated with benefit.

By “higher level” we include a patient or patient population who has a level of RBCS either at or above a reference level for a patient or patient population who have been diagnosed with cancer and are therefore in need of treatment, such as immunotherapy; or above a reference level for a normal individual or population. By “reference level” we include a level which represents a level at or above which the administration of immunotherapy will confer a clinical benefit to the patient or patient population such as overall survival, increased progression-free survival decreased risk of tumour recurrence or spread.

In one embodiment the higher level will include a patient or patient population who have a level of RBCs above the median for a patient or patient population who have been diagnosed with cancer and are therefore in need of treatment, such as immunotherapy; or it will also include a patient or patient population who have a level of RBCs which is above the median for a normal individual or patient population.

Reference levels of RBCs have been reported as:
Red Blood Cell Count (RBC) 4.3-6.2×10⁶/μL (Male)
   3.8-5.5×10⁶/μL (Female)
   3.8-5.5×10⁶/μL (Infant/Child)

### Reticulocyte Haemoglobin, Bone Marrow Iron

A reticulocyte haemoglobin content <26 pg and a percentage of hypochromic red cells >2.5 have been proposed as markers of iron-deficient erythropoiesis in such subjects. Although iron is mostly stored in the body in the haemoglobin, about 30 percent of iron is also stored as ferritin and hemosiderin in the bone marrow, spleen, and liver and this may also be used in the methods of the present invention in line with the descriptions above for e.g. other methods of measuring iron or haemoglobin which are incorporated herein.

### Hepcidin

We have identified that immunotherapy performs better in cancer patients with a certain baseline baseline levels described herein. We have now identified that this result also holds true for patients with at or below normal reference hepcidin levels. We have also found that there is an association between the level of hepcidin and the magnitude of antibody response and enhanced clinical benefit.

Hepcidin is a 25-amino acid peptide hormone produced by the liver which appears to be the master regulator of iron homeostasis in humans and other mammals. Hepcidin inhibits iron transport by binding to the iron channel ferroportin, on gut enterocytes, macrophages, and reticuloendothelial cells. Inhibiting ferroportin shuts off the iron transport out of these cells, which store iron. Hepcidin activity is partially responsible for iron sequestration seen in anaemia of chronic disease. Hepcidin is induced by IL-6 indicating that hepcidin induction by inflammation is a type II acute-phase response. Hepcidin can be measured in serum or urine by SELDI-TOF-MS using synthetic stable isotope labelled hepcidin as an internal standard (which is 10 Daltons heavier than the endogenous hepcidin). The concentration of hepcidin in a sample is calculated from the mass spectra using the peak height ratio of endogenous and labelled hepcidin. This generates a reproducible quantitation of hepcidin.

Thus, in one aspect the invention provides a method for predicting the magnitude of an antibody response to an immunotherapy in a cancer patient receiving the immunotherapy treatment which involves (a) measuring a level of hepcidin in a sample from the cancer patient, and (b) comparing the level of hepcidin in the sample to a reference level of hepcidin, wherein a lower level of hepcidin in the sample correlates with increased benefit to the patient.

By “lower level” we include a patient or patient population who has a level of hepcidin at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of hepcidin at or below the median for a patient or patient population who have been diagnosed with cancer and are therefore in need of treatment, such as immunotherapy; or at or below the median for a normal individual or patient population.

Hepcidin levels in normal reference patients have been reported for example as 17-286 ng/mL for women and 29-254 ng/mL for men.

### C-Reactive Protein

We have identified that immunotherapy performs better in cancer patients with a certain baseline factor levels described herein. We have now identified that this result also holds true for patients with at or below normal reference C-reactive protein (CRP) levels. We have also found that there is an association between the level of C-reactive protein (CRP) and the magnitude of antibody response and enhanced clinical benefit.

C-reactive protein (CRP) is a protein found in the blood, the levels of which rise in response to inflammation. As such CRP levels can be elevated in patients with anaemia of chronic disease. Normal reference concentration in healthy human serum is usually lower than 10 mg/L, slightly increasing with age. Higher levels are found in late pregnant women, mild inflammation and viral infections (10-40 mg/L), active inflammation, bacterial infection (40-200 mg/L), severe bacterial infections and burns (>200 mg/L).

Thus, in one aspect the invention provides a method for predicting the magnitude of an antibody response to an immunotherapy in a cancer patient receiving the immunotherapy treatment which involves (a) measuring a level of CRP in a sample from the cancer patient, and (b) comparing the level of CRP in the sample to a reference level of CRP, wherein a lower level of CRP in the sample correlates with increased benefit to the patient.

By “lower level” we include patients who have a level of baseline CRP at or below the median for a patient in need of immunotherapy or below or towards the lower end of normal levels.

By “lower level” we include a patient or patient population who has a level of CRP at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of CRP at or below the median for a patient or patient population who have been diagnosed with cancer and are therefore in need of treatment, such as immunotherapy; or at or below the median for a normal individual or patient population.

### Interleukin 6

We have identified that immunotherapy performs better in cancer patients with a certain baseline factor levels as described herein. We have identified that this result also holds true for patients with at or below normal reference interleukin 6 (IL-6) levels. We have also found that there is an association between the level of interleukin 6 (IL-6) and the magnitude of antibody response and enhanced clinical benefit.

IL-6 is required for the induction of hepcidin and hypoferremia during inflammation and this cytokine by itself rapidly induces hypoferremia in humans. The association between IL-6 and ACD suggests that IL-6-mediated bone marrow suppression is the main mechanism for development of ACD. IL-6 is a major product of IL-1- or TNF-stimulated cells.

Thus, in one aspect the invention provides a method for predicting the magnitude of an antibody response to an immunotherapy in a cancer patient receiving the immunotherapy treatment which involves (a) measuring a level of IL-6 in a sample from the cancer patient, and (b) comparing the level of IL-6 in the sample to a reference level of IL-6, wherein a lower level of IL-6 in the sample correlates with increased benefit to the patient.

By “lower level” we include patients who have a level of baseline IL-6 at or below the median for a patient in need of immunotherapy or below or towards the lower end of normal levels.

By “lower level” we include a patient or patient population who has a level of IL-6 at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of IL-6 at or below the median for a patient or patient population who have been diagnosed with cancer and are therefore in need of treatment, such as immunotherapy; or at or below the median for a normal individual or patient population.

### Interleukin 10

We have identified that immunotherapy performs better in cancer patients with a certain baseline factor levels described herein. We have identified that this result also holds true for patients with at or below normal interleukin 10 (IL-10) levels. We have also found that there is an association between the level of interleukin 10 (IL-10) and the magnitude of antibody response and enhanced clinical benefit.

IL-10, which is up-regulated in most inflammatory disorders of the body, alters iron metabolism in vivo by exerting a direct effect on ferritin translation and presumably subsequently storage of iron within activated monocytes/macrophages which may limit the availability of iron to erythroid progenitor cells. This induces anaemia and is thus be involved in the pathogenesis of ACD.

Thus, in one aspect the invention provides a method for predicting the magnitude of an antibody response to an immunotherapy in a cancer patient receiving the immunotherapy treatment which involves (a) measuring a level of IL-10 in a sample from the cancer patient, and (b) comparing the level of IL-10 in the sample to a reference level of IL-10, wherein a lower level of IL-10 in the sample correlates with increased benefit to the patient.

By “lower level” we include patients who have a level of baseline IL-10 at or below the median for a patient in need of immunotherapy or below or towards the lower end of normal levels.

By “lower level” we include a patient or patient population who has a level of IL-10 at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of IL-10 at or below the median for a patient or patient population who have been diagnosed with cancer and are therefore in need of treatment, such as immunotherapy; or at or below the median for a normal individual or patient population.

### Vascular Endothelial Growth Factor

We have identified that immunotherapy performs better in cancer patients with a certain baseline factor levels as described herein. We have now identified that this result also holds true for patients with at or below normal vascular endothelial growth factor (VEGF) levels. We have also found that there is an association between the level of vascular endothelial growth factor (VEGF) and the magnitude of antibody response and enhanced clinical benefit.

Vascular endothelial growth factor (VEGF), is a potent angiogenic cytokine and plays a major role in tumour vessel formation. VEGF can be induced locally by hypoxia and has been shown to be induced systemically by anaemia.

Thus, in one aspect the invention provides a method for predicting the magnitude of an antibody response to an immunotherapy in a cancer patient receiving the immunotherapy treatment which involves (a) measuring a level of VEGF in a sample from the cancer patient, and (b) comparing the level of VEGF in the sample to a reference level of VEGF, wherein a lower level of VEGF in the sample correlates with increased benefit to the patient.

By “lower level” we include patients who have a level of baseline VEGF at or below the median for a patient in need of immunotherapy or below or towards the lower end of normal levels.

By “lower level” we include a patient or patient population who has a level of VEGF at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of VEGF at or below the median for a patient or patient population who have been diagnosed with cancer and are therefore in need of treatment, such as immunotherapy; or at or below the median for a normal individual or patient population.

### Tumour Necrosis Factor Alpha and Interleukin 1

We have identified that immunotherapy performs better in cancer patients with a certain baseline factor levels as described herein. We have identified that this result also holds true for patients with at or below normal interleukin 1 (IL-1) and tumour necrosis factor alpha (TNF-α) levels. We have also found that there is an association between the level of interleukin 1 (IL-1) and tumour necrosis factor alpha (TNF-α) and the magnitude of antibody response and enhanced clinical benefit.

Interleukin-1 and TNF-alpha are pro-inflammatory cytokines and act synergistically. Whether induced by an infection, trauma, ischemia, immune-activated T cells, or toxins, IL-1 and TNF-α initiate the cascade of inflammatory mediators by targeting the endothelium. They are able to induce hypoferremia by modulating macrophage iron metabolism. This is primarily exerted by an effect of the cytokines on the expression of the iron storage protein ferritin.

Thus, in one aspect the invention provides a method for predicting the magnitude of an antibody response to an immunotherapy in a cancer patient receiving the immunotherapy treatment which involves (a) measuring a level of IL-1 and TNF-α in a sample from the cancer patient, and (b) comparing the level of IL-1 and TNF-α in the sample to a reference level of IL-1 and TNF-α, wherein a lower level of IL-1 and TNF-α in the sample correlates with increased benefit to the patient.

By “lower level” we include patients who have a level of baseline IL-1 and TNF-α at or below the median for a patient in need of immunotherapy or below or towards the lower end of normal levels.

By “lower level” we include a patient or patient population who has a level of IL-1 and TNF-α at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of IL-1 and TNF-α at or below the median for a patient or patient population who have been diagnosed with cancer and are therefore in need of treatment, such as immunotherapy; or at or below the median for a normal individual or patient population.

### Interferon Gamma

We have identified that immunotherapy performs better in cancer patients with a certain baseline factor levels as described herein. We have now identified that this result also holds true for patients with at or below normal Interferon gamma (IFN-γ) levels. We have also found that there is an association between the level of Interferon gamma (IFN-γ) and the magnitude of antibody response and enhanced clinical benefit.

Interferon gamma (IFN-γ) or type II interferon, is a cytokine that is critical for innate and adaptive immunity against viral and intracellular bacterial infections and for tumour control. IFN-γ can up regulates the expression of the divalent metal transporter 1 (DMT1) on activated macrophages and increases their iron uptake. IFN-γ also has a direct effect on the proliferation of erythroid progenitor cells causing a suppression of erythropoiesis.

Thus, in one aspect the invention provides a method for predicting the magnitude of an antibody response to an immunotherapy in a cancer patient receiving the immunotherapy treatment which involves (a) measuring a level of IFN-γ in a sample from the cancer patient, and (b) comparing the level of IFN-γ in the sample to a reference level of IFN-γ, wherein a lower level of IFN-γ in the sample correlates with increased benefit to the patient.

By “lower level” we include patients who have a level of baseline IFN-γ at or below the median for a patient in need of immunotherapy or below or towards the lower end of normal levels.

By “lower level” we include a patient or patient population who has a level of IFN-γ at or below a reference level for a patient or patient population who have been diagnosed with cancer and are therefore in need of treatment, such as immunotherapy; or at or below a reference level for a normal individual or population. By “reference level” we include a level which represents a level at or below which the administration of immunotherapy will confer a clinical benefit to the patient or patient population, such as improved overall survival, increased progression-free survival, decreased risk of tumour recurrence or spread.

In one embodiment the lower level will include a patient or patient population who has a level of IFN-γ at or below the median for a patient or patient population who have been diagnosed with cancer and are therefore in need of treatment, such as immunotherapy; or at or below the median for a normal individual or patient population.

In addition to the foregoing, the invention includes, as an additional aspect, all embodiments of the invention narrower in scope in any way than the variations specifically mentioned above. For example, although aspects of the invention may have been described by reference to a genus or a range of values for brevity, it should be understood that each member of the genus and each value or sub-range within the range is intended as an aspect of the invention. Likewise, various aspects and features of the invention can be combined, creating additional aspects which are intended to be within the scope of the invention. Although the applicant(s) invented the full scope of the claims appended hereto, the claims appended hereto are not intended to encompass within their scope the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

FIG. 1: Pre-treatment 5T4 (1a) and MVA (1b) antibody levels in healthy donors (normal; n=50) compared to RCC patients classified as good prognosis (MSKCC=0; n=419) or intermediate prognosis (MSKCC=1-2; n=305). The figure plots the mean pre-treatment antibody levels (±0.95 CI) and shows the P-value (Mann-Whitney) between groups.

FIG. 2: Analysis of the magnitude of 5T4 and MVA antibody responses in patients classified as having sero-converted. The figures plot the mean (±0.95 CI) 5T4 (2a) and MVA (2b) antibody responses (by SOC) in patients treated with MVA-5T4 who were classified as having mounted a positive 5T4 antibody response.

FIG. 3: Quantitative relationship between IRS and survival in MVA-5T4 (TroVax®) treated patients (TRIST). The figure shows that patients who have a higher IRS prior to treatment with MVA-5T4 (TroVax®) show a significantly increased survival benefit.

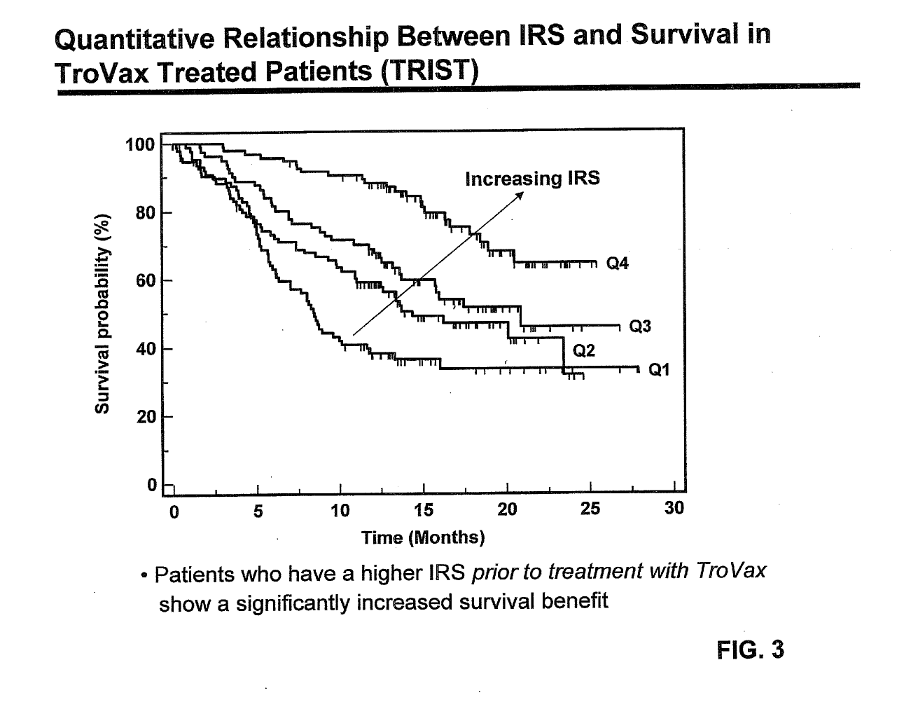

FIG. 4: Cumulative data from Phase II: IRS and survival in MVA-5T4 (TroVax®) treated patients. The data is from 9 studies, 108 patients in CRC, RCC and prostate cancer. The cumulative Phase II data mimics the TRIST data reported in FIG. 3.

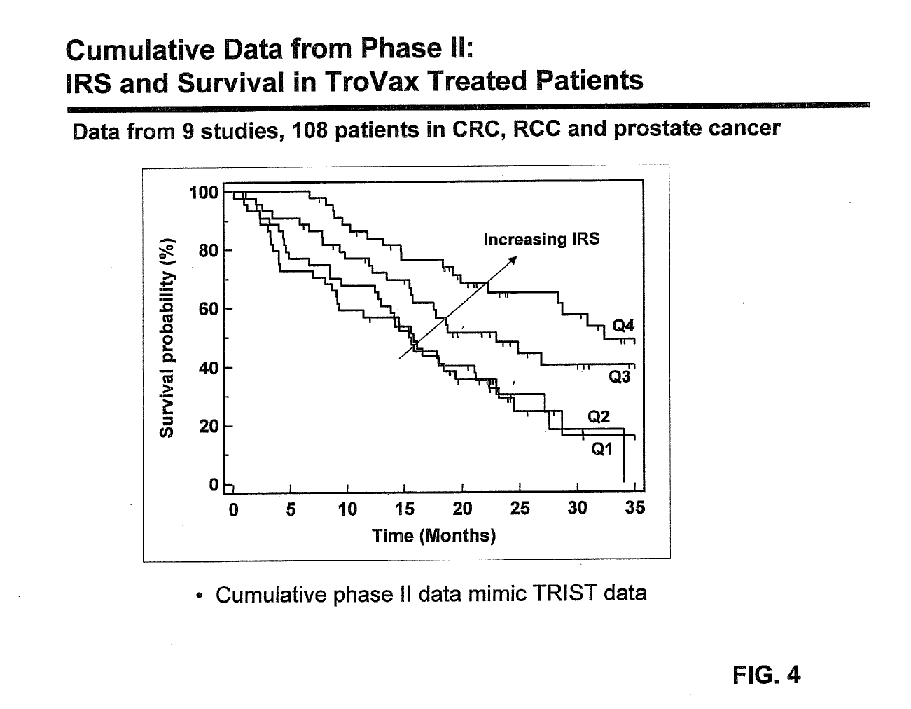

FIG. 5: Direct Predictors of Treatment Benefit (Immunologically Evaluable Dataset; n=590).

FIG. 6: Direct Predictors of Treatment Benefit (ITT Patient population; n=733).

### DETAILED DESCRIPTION

We describe a detailed analysis of antibody responses for the 5T4 tumour associated antigen and the MVA viral vector in >550 cancer patients treated with the investigational vaccine MVA-5T4 (TroVax®) or standard of care (placebo). However, the results may be applied to other immunotherapy treatments and cancer types and the following should be read in such general terms. As the same factors identified herein as being predictive of treatment benefit also fit the data from phase II studies of TroVax in colorectal and prostate cancer, and as these factors are also associated with anaemia of chronic disease which is associated with many different types of cancer (Weiss, G, Goodnough, LT, New England Journal of Medicine (2005) 352: 1011-1023) we consider that these factors will be applicable to other immunotherapy treatments and cancer types and the following should be read in such general terms.

The discussion in this section is merely illustrated by way of ease of reference to the MVA-5T4 and RCC.

We found that quantification of antibody levels to a tumour associated antigen prior to the start of treatment with MVA-5T4 showed that RCC patients had elevated 5T4 and MVA antibody levels relative to healthy donors. Previously, we have reported data from phase II trials which demonstrated that patients with colorectal, prostate and renal cancer had elevated 5T4 antibody levels relative to healthy donors (6). It is possible that the elevated MVA antibody levels see in RCC patients compared to healthy donors is due to the differential in average age (58 compared to 34) which is likely to reflect prior exposure to smallpox vaccine. It would be logical to conclude that patients with renal cancer have 5T4 expressed on their tumours which drives the production of auto-antibody responses. It is interesting to note that patients classified as having an intermediate prognosis had significantly higher 5T4 antibody levels compared to those with a good prognosis suggesting that the more advanced the disease stage the higher the antibody response. This may fit with the observation that 5T4 protein appears to be even more highly expressed on late stage disease and has been shown to have prognostic value in some cancers. In contrast, no differential in MVA antibody levels were seen between patients classified as good or intermediate prognosis.

Following treatment with MVA-5T4, the magnitude of 5T4-specific antibody responses in patients classified as having sero-converted was largely comparable across all 3 standards of care (SOCs), although there was a trend towards higher responses in patients receiving cytokine therapy.

Few immunotherapy studies have demonstrated convincingly that there is a direct link between the predicted mode of action of an agent and therapeutic benefit. Phase I and II results for MVA-5T4 in renal, colorectal and prostate cancer patients were encouraging and demonstrated that immune responses were induced in almost all treated patients and associations between 5T4-specific cellular or humoral responses and clinical benefit were reported in seven of the nine phase II studies (7-15). In particular, studies in RCC and colorectal cancer patients have demonstrated an association between 5T4-specific (but not MVA) antibody responses and enhanced survival (8, 11, 13).

This study provides additional strong evidence of an association between 5T4-specific immune response and clinical benefit. We have also derived a surrogate for immune response using the MVA-5T4 treated subjects and applying this to a survival analysis in MVA-5T4 and placebo treated patients to evaluate treatment benefit.

The surrogate is also a prognostic factor for survival in both placebo and MVA-5T4 treated groups (in the latter, the surrogate for immune response is a better prognostic factor than the immune response itself). The difference in the hazard ratios between patients treated with placebo and those receiving MVA-5T4 is highly significant: the higher the value of the surrogate the lower the value of the MVA-5T4 hazard relative to placebo. The demonstration that the immune response surrogate is a predictor of treatment benefit has several important implications:

(1) it is indirect evidence of the therapeutic activity of MVA-5T4;

(2) it establishes that some subsets of patients are more likely to obtain clinical benefit from MVA-5T4 than other subsets;

(3) it further confirms that the 5T4 immune response at week 10 is an early marker of efficacy.

To our knowledge, this is the first report in the cancer vaccine field in which it has been demonstrated convincingly that an antigen-specific immune response induced by vaccination is associated with enhanced patient survival and is not simply a function of the general “health” of a patient.

It is interesting and unexpected that haemoglobin and haematocrits in combination should be identified as factors contributing to the prediction of immune response. An explanation of the mechanistic rationale to explain this is not immediately obvious. However, low levels of haemoglobin and haematocrits are indicators of anaemia which is often caused by iron deficiency and is commonly seen in renal cancer patients. Although there are contradictory data regarding the role of iron in adaptive immune responses, some publications have reported that iron deficiency can impact on the quality and/or quantity of immune responses (16, 17). Indeed, it has been demonstrated that iron insufficiency can inhibit the phenotypic maturation of DCs, leading to reduced T cell activation (18). Furthermore, vaccinia virus is known to induce innate immunity and dendritic cell (DC) maturation through stimulation of toll-like receptors (TLR) 2, 3 and 4 (19, 20) and haemoglobin has recently been shown to synergize with TLR agonists to potentiate innate immune responses and cytokine release (21). Therefore, perhaps, in patients with higher baseline haemoglobin levels, immune responses may be potentiated by the synergy of haemoglobin for TLR stimulation at the site of injection, while those with lower haemoglobin (or iron) levels may have dysfunctional immune responses.

In light of the unexpected constituents of the IRS, it was very encouraging that the surrogate showed a significant association with both immune response and survival when applied to data collated from phase I and II studies of MVA-5T4 in renal, colorectal and prostate cancer patients. The ability of the immune response surrogate to predict 5T4 immune response in our phase II renal cancer dataset provides some validation of the algorithm. Furthermore, the successful application of the surrogate to colorectal and prostate cancer patients treated with MVA-5T4 suggests that it may have application beyond renal cancer.

By their very nature, immunotherapy products will have a delayed therapeutic benefit leading to protracted monitoring before clinical benefit (or lack thereof) is detectable. The availability of an early marker of efficacy would be particularly beneficial for cancer immunotherapy products. Here, we have confirmed previous observations that an antibody response specific for 5T4 and generated within 10 weeks of treatment initiation is associated with enhanced survival. This represents the identification of an early marker of efficacy for the cancer vaccine MVA-5T4.

In conclusion, we have shown that patients with a higher value for a surrogate of immune response obtain greater benefit from MVA-5T4. This finding is an indirect confirmation of the therapeutic activity of MVA-5T4 and has important implications for future clinical trials which will target patients with good performance status and minimize the recruitment of patients with abnormal levels of various hematology factors.

### Immunotherapy

It is contemplated that the methods of the invention are suitable and applicable for all viral vectors and non-viral vectors adaptable for delivery of an exogenous gene to a mammalian cell for expression of the gene in the cell. A variety of viral vectors may be employed as disclosed herein. In some preferred aspects, viral vectors are replication deficient. Furthermore, as detailed above, a viral vector may preferably comprise poxvirus such as a vaccinia viral vector. A variety of vaccinia viral vectors are known in the art in certain aspects a vaccinia viral vector for use herein may be a modified vaccinia Ankara (MVA) virus. Other exemplary immunotherapies include compositions that include the protein antigen itself; or fragments or epitopes of the antigen; or vectors for delivering a transgene that encodes the antigen (e.g., plasmid or liposomal vectors).

In other aspects the immunotherapy may comprise a viral vector which can be either the same as the original vector or a different viral vector such as a heterologous prime-boost vaccination regimen. Heterologous prime-boost vaccination regimens have been previously described in, for example, PCT publication WO 98/56919.

In some aspects immunotherapeutic methods concern a maintenance immunotherapy that does not comprise the viral vector. For instance, the maintenance immunotherapy comprises a composition comprising the antigen, or at least one epitope thereof, and an adjuvant or carrier. For example, a maintenance immunotherapy may comprise a plasmid that contains a nucleotide sequence that encodes the antigen, operably linked to an expression control sequence to permit expression of the antigen in cells of the mammalian subject. Such a method may, optionally, further comprise (d) immunizing the subject having a measurable immune response to the antigen with a maintenance immunotherapy that is free from the viral vector. Exemplary maintenance immunotherapies include compositions that include the protein antigen itself; or fragments or epitopes of the antigen; or vectors for delivering a transgene that encodes the antigen (e.g., plasmid or liposomal vectors).

In other aspects the maintenance immunotherapy may comprise a viral vector which can be either the same as the original vector or a different viral vector such as a heterologous prime-boost vaccination regimen.

Likewise, methods disclosed herein are applicable to immunotherapy utilizing a variety of antigens. In certain aspects, an antigen as defined herein comprises at least one tumour antigen such as the tumour antigens listed in the detailed description below. For example, the tumour antigen may comprise a 5T4 antigen. 5T4 antigen and viral vectors comprising 5T4 have been previously described for example in U.S. Pat. No. 7,148,035, incorporated herein by reference.

In one embodiment the invention is intended to be applicable to immunotherapies directed against malignancies. Thus, in some variations, the mammalian subject for immunotherapy is a subject having a cancer such as a cancer that expresses a least one tumour antigen (tumour associated antigen). Preferably, a subject having cancer comprises a cancer which expresses the same antigen that is comprised in the viral vector used for immunotherapy. In some cases a subject comprising a cancer may be a subject with a renal cell, prostate or a colorectal cancer. Preferably a mammalian subject is a human subject.

In addition to the immunotherapy methods described herein subjects may further be treated with one or more additional therapies such as a therapy considered the standard of care for a particular disease such as cancer. For example, the additional therapy or standard of care therapy may be chemotherapy, radiation therapy, surgery or cytokine therapy.

Generally, an immunotherapy for use herein will be formulated in a pharmaceutically acceptable carrier, and may additionally comprise preservatives, salts and/or adjuvants.

I. Tumour-Associated Antigens (TAAs)

In certain aspects the application concerns a tumour associated antigen. A suitable tumour associated antigen (TAA) or tumour antigens includes 5T4. As used herein the terms tumour associated antigen and tumour antigen are used interchangeably. Other suitable antigens include TAAs in the following classes: cancer testis antigens (e.g., HOM-MEL-40), differentiation antigens (e.g., HOM-MEL-55), overexpressed gene products (HOM-MD-21), mutated gene products (NY-COL-2), splice variants (HOM-MD-397), gene amplification products (HOM-NSCLC-11) and cancer related autoantigens (HOM-MEL-2.4) as reviewed in Cancer Vaccines and Immunotherapy (2000) Eds Stern, Beverley and Carroll, Cambridge University Press, Cambridge. Further examples include, MART-1 (Melanoma Antigen Recognized by T-cells-1) MAGE-A (MAGE-A1, MAGE-A2, MAGE-A3, MAGE-A4, MAGE-A6, MAGE-A8, MAGE-A10, MAGE-A12), MAGE B (MAGE-B1-MAGE-B24), MAGE-C (MAGE-C1/CT7, CT10), GAGE (GAGE-1, GAGE-8, PAGE-1, PAGE-4, XAGE-1, XAGE-3), LAGE (LAGE-1a(1S), -1b(1L), NY-ESO-1), SSX (SSX1-SSX-5), BAGE, SCP-1, PRAME (MAPE), SART-1, SART-3, CTp11, TSP50, CT9/BRDT, gp100, MART-1, TRP-1, TRP-2, MELAN-A/MART-1, Carcinoembryonic antigen (CEA), prostate-specific antigen (PSA), MUCIN (MUC-1) and Tyrosinase. TAAs are reviewed in Cancer Immunology (2001) Kluwer Academic Publishers, The Netherlands. Additional tumour associated antigens include Her 2, survivin and TERT.

The term “antigen” refers to protein or peptide to be introduced into a subject. As described herein, an antigen may be provided through delivering a peptide or protein or through delivering a nucleic acid encoding a peptide or protein.

By “antigen” in the context of the present invention it is also meant to incorporate an antigenic peptide derived from an antigen. In particular, “tumour associated antigen” is intended to encompass a peptide derived from a tumour associated antigen.

An antigen such as a tumour associated antigen can be provided for use as a medicament in a number of different ways. It can be administered as part of a viral vector. A number of suitable viral vectors will be familiar to those skilled in the art and include a number of vectors described herein.

II. TroVax® Vaccine

TroVax® consists of a highly attenuated strain of vaccinia virus (VV), termed Modified Vaccinia Ankara, (MVA), and contains the human TAA 5T4 glycoprotein gene under regulatory control of a modified promoter, mH5. Thus, by “TroVax®” we include Modified Vaccinia Ankara, (MVA), that contains the human TAA 5T4 glycoprotein gene, and preferably under regulatory control of a modified promoter, mH5

MVA was developed as a safe vaccine for smallpox and MVA was derived from the VV Ankara strain by passaging in primary chick embryo fibroblasts (CEF), after which it was found to be replication defective in all mammalian cell lines tested, except Baby Hamster Kidney cells (BHK-21). Molecular genetic analysis of MVA has revealed substantial differences from the replication competent vaccinia virus which indicate that reversion of attenuation is highly unlikely. MVA is non-pathogenic in mammals including suckling mice, rabbits and primates. Importantly, no complications were reported when MVA was administered to over 120,000 subjects, many of whom were at risk from vaccine complications. Replication of competent strains of VV are handled in a Biosafety level II environment; however, MVA has been assigned Biosafety level I status by the National Institutes of Health Intramural Biosafety Committee in the US, the UK Health and Safety Executive and the biosafety authorities in Germany.

5T4 is a 72 kDa oncofoetal glycoprotein that is expressed on over 70% of carcinomas of the kidney, breast, gastrointestinal tract, colon and ovaries. Unlike other self-antigen TAAs such as CEA, 5T4 expression as detected by histochemical staining appears to be tumour specific with only low level sporadic staining observed in the gut and pituitary. However this level of staining is so low that it is difficult to determine if it is specific. 5T4-positive tumours include invasive carcinoma of the Ampulla of Vater, breast, colon, endometrium, pancreas, or stomach; a squamous carcinoma of the bladder, cervix, lung or oesophagus; a tubulovillous adenoma of the colon; a malignant mixed Mullerian tumour of the endometirem; a clear cell carcinoma of the kidney; a lung cancer (large cell undifferentiated, giant cell carcinoma, broncho-alveolar carcinoma, metastatic leiomyosarcoma); an ovarian cancer (a Brenner tumour, cystadenocarcinoma, solid teratoma); a cancer of the testis (seminoma, mature cystic teratoma); a soft tissue fibrosarcoma,; a teratoma (anaplastic germ cell tumours); or a trophoblast cancer (choriocarcimoma (e.g. in uterus, lung or brain), tumour of placental site, hydatidiform mole). Immunohistochemical analysis indicates that 5T4 expression is an indicator of poor prognosis in colorectal cancer. Additionally, when tumour cells are transfected with the cDNA encoding 5T4, they display increased motility suggesting that expression of this molecule may induce metastatic properties in a tumour.

TroVax® is able to induce an anti-5T4 antibody response in mice. Additionally, such a response is able to prevent the establishment of syngeneic tumour cells expressing human 5T4 in two murine tumour models. To model more accurately the possible anti-tumour effects of TroVax® in humans, MVA recombinants were constructed expressing the murine homologue of 5T4 (m5T4). In this self-antigen model MVA-m5T4 induction of an m5T4 antibody response was observed. Furthermore such a response is able to retard or prevent the establishment of syngeneic tumour cells expressing m5T4. Mice have been vaccinated on four occasions with MVA-m5T4 and there have been no reports of toxicity. In addition a number of studies have explored the toxicological consequences of immunization with TroVax®. Mice have been immunized with up to 12 repeated administrations of TroVax®. There were no TroVax® related deaths or adverse effects on clinical signs, body weight, food consumption, organ weights or clinical pathology. There were no macroscopic or microscopic findings suggestive of systemic toxicity due to the test articles.

Because 5T4 is an oncofoetal antigen, mice, previously vaccinated with MVA-m5T4, were used for breeding. It was found that immunity to m5T4 did not have a detrimental effect on the ability of mice to become pregnant or give birth to healthy progeny. In a more detailed study, female mice were administered with approx 107 pfu of TroVax® or MVA-m5T4 or placebo at 21 and 14 days prior to pairing with untreated males and, for the pregnant females, on Day 6 of gestation. The pregnant females were maintained to Day 18 of gestation then the injected animals and their respective foetuses analysed macroscopically at necropsy. All clinical observations and necropsy findings were unremarkable. The pregnancy rate was slightly lower in the groups given both TroVax® and MVA-m5T4 compared to control. The toxicological significance of this finding is uncertain but may reflect a treatment impact on mating behavior. There was no adverse effect of treatment with either TroVax® or MVA-m5T4 on the uterine/implantation or foetal data. In summary, there was no female or maternal toxicity and no embryo-foetal toxicity in either group. Histological examination of the tissues from the MVA-m5T4 animals revealed no adverse microscopic findings.

It is apparent from pre-clinical studies that TroVax® has little potential to induce toxicity but is likely to induce an efficacious immune response to 5T4. In vivo studies suggest that such an immune response will have anti-tumour activity.

TroVax® has been administered to over 400 patients with metastatic colorectal or renal cancer. Over 3000 doses have been administered. No serious adverse event attributed to TroVax® by investigators or the sponsor has been reported. Mild transient injection site reactions are reported in the majority of patients together with mild transient pyrexia. No other notable, common or serious adverse events have been reported in studies using TroVax® as a single agent in heavily pretreated patients or in studies combining TroVax® with chemotherapy, (5FU and leucovorin combined with either oxaliplatin or irinotecan), interferon-a , IL-2 (high dose intravenous regimen or low dose subcutaneous injections) or with sunitinib.

### Cancer Treatment

The present invention is particularly suitable for use in predicting the response to the aforementioned immunotherapeutic agents in patients or patient population with a cancer. Such cancers include, for example, non-solid tumours such as leukaemia, multiple myeloma or lymphoma, and also solid tumours, for example bile duct, bone, bladder, brain/CNS, breast, colorectal, cervical, endometrial, gastric, head and neck, hepatic, lung, muscle, neuronal, oesophageal, ovarian, pancreatic, pleural/peritoneal membranes, prostate, renal, skin, testicular, thyroid, placental, uterine and vulval tumours.

The present invention is particularly suitable for identifying those patients with renal, colorectal, breast, prostate or ovarian cancer, more particularly renal or colorectal cancer that will respond to treatment with immunotherapeutic agents, such as TroVax® as hereinbefore defined.

### Sample

As used herein, the term “sample” is intended to mean any biological fluid, cell, tissue, organ or portion thereof. The term includes samples present in an individual as well as samples obtained or derived from the individual. For example, a sample can be a histologic section of a specimen obtained by biopsy, or samples that are placed in or adapted to tissue culture. Furthermore a sample can be a subcellular fraction or extract, or a crude or substantially pure protein preparation.

In the methods of the invention, a sample can be, for example, a cell or tissue obtained using a biopsy procedure or can be a fluid sample containing cells, such as blood, serum, plasma, semen, urine, or stool. Those skilled in the art will be able to determine an appropriate sample, which will depend on cancer type, and an appropriate method for obtaining a biopsy sample, if necessary. When possible, it can be preferable to obtain a sample from a patient using the least invasive collection means. For example, obtaining a fluid sample from a patient, such as blood, saliva, serum, plasma, semen, urine or stool, is less invasive than collecting a tissue sample.

It will be appreciated that the measurements taken in relation to the present invention can be made used the same or different samples. The samples can be taken at different times, but preferably the same sample will be used. More preferably the sample is one taken prior to the start of immunotherapy treatment.

### Reference Level

As used herein, the term “reference level” refers to a control level of expression of a factor or biomarker used to evaluate a test level of expression of a factor or biomarker in a sample of a patient. For example, when the level of haemoglobin in the patient is higher than the reference level of haemoglobin, the patient will be considered to have a high level of haemoglobin. Conversely, when the level of haemoglobin in the patient is lower than the reference level, the patient will be considered to have a low level of haemoglobin. In a further example, when the level of iron in the patient is higher than the reference level of iron, the patient will be considered to have a high level of iron. In some variations, the reference level may be a range or an average or median measurement of a biomarker calculated from a plurality mammalian subjects that are proposed for the immunotherapy or are not in need of immunotherapy. Where the reference is an average or median, a measurement for the biomarker level above the reference measurement is scored as elevated and a measurement below the reference measurement is scored as reduced. When the reference is a range a measurement for the biomarker around or above the top level for the reference measurement is scored as elevated and a measurement around or below the bottom level for the reference measurement is scored as reduced. In other variations, a measurement that statistically varies from the median or mean by a suitable significant amount (e.g., 1 or 1.5 or 2 standard deviations; or by a “p-value” or other statistical measure of significance) is scored as elevated or reduced.

In other variations, a reference level may be a baseline measurement or any other absolute measurement for a particular assay tool. In such variations, an elevated level or a reduced level may represent values that are a certain multiple or fraction of the reference value.

Some aspects of the invention involve screening for or determining the presence of a measurable difference from the reference level. Measurable may be defined as a level greater or lower than a baseline response, and more preferably at least about 2, 5, 10, 50, 100 or 1000 fold over or below a baseline response. In cases where there is no measurable baseline response, a baseline response may be defined as the lower detection limit of the assay used to measure the level.

The reference level can be determined by a plurality of methods, provided that the resulting reference level accurately provides a level of a biomarker above which exists a first group of patients having a different probability of survival than that of a second group of patients having levels of the biomarker below the reference level or vice versa. The reference level can be determined by, for example, measuring the level of a biomarker in a non-tumourous sample as the sample of the patient to be tested. The reference level can also be a level of a biomarker of in vitro cultured cells which can be manipulated to simulate tumour cells, or can be manipulated in any other manner which yields levels of the biomarker which accurately determine the reference level.

The reference level can also be determined by comparison of the level of a biomarker, such as antibodies to tumour associated antigens, haemoglobin or haematocrit, in populations of patients having the same cancer. This can be accomplished, for example, by histogram analysis, in which an entire cohort of patients are graphically presented, wherein a first axis represents the level of the biomarker, and a second axis represents the number of patients in the cohort whose samples contain the biomarker at a given level. Two or more separate groups of patients can be determined by identification of subsets populations of the cohort which have the same or similar levels of the biomarker. Determination of the reference level can then be made based on a level which best distinguishes these separate groups. A reference level also can represent the levels of two or more markers. Two or more markers can be represented, for example, by a ratio of values for levels of each biomarker.

The reference level can be a single number, equally applicable to every patient, or the reference level can vary, according to specific subpopulations of patients. For example, older men might have a different reference level than younger men for the same cancer, and women might have a different reference level than men for the same cancer. Furthermore, the reference level can be some level determined for each patient individually. For example, the reference level might be a certain ratio of a biomarker in the neoplastic cells of a patient relative to the biomarker levels in non-tumour cells within the same patient. Thus the reference level for each patient can be proscribed by a reference ratio of one or more biomarkers.

The reference level may be determined by measuring the baseline level of a biomarker prior to the commencement of immunotherapy

A high level of a biomarker, such as haematocrit, platelet levels or haemoglobin can be related to a level of the biomarker above a determined reference level. Thus, a reference or basal level of a biomarker, such as platelet levels or haemoglobin in a sample is identified as a “cutoff” value, above which there is a significant correlation between the presence of the biomarker and increased or decreased tumour recurrence or spread. Those of skill in the art will recognize that some “cutoff” values are not sharp in that clinical correlations are still significant over a range of values on either side of the cutoff; however, it is possible to select an optimal cutoff value (for example varying H-scores, and the like) of a level of a biomarker for a cancer cell type. It is understood that improvements in optimal cutoff values could be determined, depending on the sophistication of statistical methods used and on the number and source of samples used to determine reference or basal values.

Verification that the reference level distinguishes the likelihood of tumour recurrence or spread in cancer patients expressing below-reference biomarker levels versus cancer patients expressing above-reference biomarker levels can be carried out using single variable or multi-variable analysis. These methods determine the likelihood of a correlation between one or more variables and a given outcome. In the specific case, the methods will determine the likelihood of a correlation between a biomarker level, or the levels of more than one biomarker (or a biomarker level coupled with another variable) and disease-free or overall survival of cancer patients. Any one of a plurality of methods well known to those of ordinary skill in the art for carrying out these analyses can be used. Examples of analysis are the Kaplan-Meier method, the log-rank test or the Cox proportional-hazards regression model.

The Kaplan-Meier estimator used in the Examples (also known as the product limit estimator) estimates the survival function from life-time data. In medical research, it might be used to measure the fraction of patients living for a certain amount of time after treatment. The survival function, also known as a survivor function or reliability function, is a property of any random variable that maps a set of events, usually associated with mortality or failure of some system, onto time. It captures the probability that the system will survive beyond a specified time. The term reliability function is common in engineering while the term survival function is used in a broader range of applications, including human mortality.

Population-based determination of reference levels, for example, by histogram analysis can be carried out using a cohort of patients sufficient in size in order to determine two or more separate groups of patients having different biomarker levels. Typically, such a cohort comprises at least 25 patients, such as at least 50 patients, including at least 75 patients, and at least 100 patients. Similarly, verification of determined reference levels can also comprise at least 25 patients, such as at least 50 patients, including at least 75 patients, and at least 100 patients.

Further, while a reference level can separate two groups of patients, it is within the scope of the invention that numerous reference values might exist which separate a plurality of populations. For example, two reference values can separate a first group of patients with high levels of a biomarker from a second group of patients with intermediate levels the biomarker, and from a third group of patients with low levels of the biomarker. The number of different reference levels can be sufficient to proscribe a curve, such as a continuous line, which describes the likelihood of disease-free or overall survival in a patient as a function of the biomarker level in that patient. Such a curve will constitute a “continuous” biomarker level, where the likelihood of disease-free or overall survival in a patient is proportional to the biomarker level in that patient. Two or more biomarker levels can also be represented by such a curve.

### Further Combinations

The reference level of the biomarkers identified herein can further be used in conjunction with another variable found to be a statistically significant indicator of the likelihood of disease-free or overall survival for cancer. Such indicators include the presence or levels of known cancer markers, or can be clinical or pathological indicators (for example, age, tumour size, tumour histology, clinical stage, family history and the like). For example, clinical stage of the cancer is also a statistically significant indicator of disease-free or overall survival, wherein the reference level of a biomarker can vary according to the clinical stage of the cancer. Thus, in one embodiment the present invention provides a further method which involves comparing platelets to a reference level wherein a low level of platelets in said sample correlates with increased benefit to said patient.

In one embodiment, the platelet level or count associated with a more favourable outcome is about ≦400×10⁹/L, about ≦350×10⁹/L, or about ≦300×10⁹/L. More particularly, the platelet level associated with a more favourable outcome is about ≦287×10⁹/L. In a preferred embodiment the baseline platelet count is about ≦281×10⁹/L, ≦281.5×10⁹/L, about ≦275.5×10⁹/L, about ≦273×10⁹/L, about ≦250×10⁹/L, ≦232×10⁹/L, about ≦225×10⁹/L, or about ≦215×10⁹/L. Especially preferred are platelet levels of ≦287×10⁹/L, ≦281×10⁹/L, or even more preferred ≦232×10⁹/L. These levels may be particularly associated with patients with RCC or CRC.

In one embodiment, the platelet level associated with a more favourable outcome in RCC is about ≦400×10⁹/L, about ≦350×10⁹/L, or about ≦300×10⁹/L. More particularly, the platelet level associated with a more favourable outcome in RCC is about ≦287×10⁹/L. In a preferred embodiment the baseline platelet count in RCC is about ≦281×10⁹/L, ≦281.5×10⁹/L, about ≦275.5×10⁹/L, about ≦273×10⁹/L, about ≦250×10⁹/L, ≦232×10⁹/L, about ≦225×10⁹/L, or about ≦215×10⁹/L. Especially preferred are platelet levels associated with RCC of ≦287×10⁹/L, ≦281×10⁹/L, or even more preferred ≦232×10⁹/L.

The above referenced platelet levels are reported in connection with a patient population diagnosed with RCC in which the platelet levels ranged from 114 to 1074×10⁹/L and the median platelet level is 281×10⁹/L; an RCC patient population and a CRC patient population in which the median platelet levels were 273×10⁹/L, but the levels or their equivalents in relation to other patient populations may be generally applicable to the field of immunotherapy.

A normal platelet count in a healthy person (1+ year) is between 130,000 and 400,000 per mm³ (microlitre) of blood (130-400×10⁹/L).

### Survival

One aspect of the present invention is seeking to achieve improved patient survival including progression-free survival, disease free-survival and overall survival, and also reducing the risk of recurrence and/or metastases. As used herein, the term “disease-free survival” includes the lack of tumour recurrence and/or spread and the fate of a patient after diagnosis, for example, a patient who is alive without tumour recurrence. The phrase “overall survival” refers to the fate of the patient after diagnosis, regardless of whether the patient has a recurrence of the tumour.

As used herein, the term “risk of recurrence” refers to the probability of tumour recurrence or spread in a patient subsequent to diagnosis of cancer, wherein the probability is determined according to the process of the invention.

Tumour recurrence refers to further growth of neoplastic or cancerous cells after diagnosis of cancer. Particularly, recurrence can occur when further cancerous cell growth occurs in the cancerous tissue. Tumour spread refers to dissemination of cancer cells into local or distant tissues and organs, for example during tumour metastasis. Tumour recurrence, in particular, metastasis, is a significant cause of mortality among patients who have undergone surgical treatment for cancer. Therefore, tumour recurrence or spread is correlated with progression-free survival, disease-free and overall patient survival.

Progression-free survival denotes the chances of staying free of disease progression after a particular treatment.

### Benefit

As used in the context of a course of treatment, “benefit” refers to the ability of the course of treatment to decrease the risk of tumour recurrence or spread and therefore to increase the likelihood of disease-free, progression-free, or overall survival of the patient.

The methods of the invention for determining a prognosis for survival for a cancer patient are applicable to patients at any stage of tumour progression, and further can be used to determine a stage of tumour progress. A stage of a tumour refers to the degree of progression of a tumour. Various stages of tumour development are well known to those of skill in the art, as exemplified in Markman, “Basic Cancer Medicine,” Saunders, (ed. Zorab, R.) (1997). For example, cancers can be staged into three general stages--localized, regional spread, and distant spread. Cancers also can be staged using the TNM Classification of Malignant Tumours (TNM), which considers the extent of direct spread within affected and nearby tissues, the extent of spread to nearby lymph nodes, and the extent of spread to distant organs. Based on these features, spread of cancers can be summarized by assigning Roman numerals from 0 through IV. Those skilled in the art can select an appropriate staging system for a particular type of cancer.

In particular, colon cancer can be staged using the Dukes, Astler-Coller and AJCC (American Joint Committee on Cancer)/TNM systems, which describe the spread of the cancer in relation to the layers of the wall of the colon or rectum, organs next to the colon and rectum, and other organs farther away. Dukes stage A is equivalent to AJCC/TNM stage I and Astler-Coller stage A, BI; Duke's stage B is equivalent to AJCC/TNM stage II and Astler-Coller stage B2, B3. Dukes stage C is equivalent to AJCC/TNM stage III and Astler-Coller stage C1, C2, C3. AJCC/TNM stages of colorectal cancer are as follow: Stage 0: the cancer has not grown beyond the inner layer (mucosa) of the colon or rectum. This stage is also known as carcinoma in situ or intramucosal carcinoma; Stage I: the cancer has grown through the mucosa into the submucosa, or can also have grown into the muscularis propria, but it has not spread outside the wall itself into nearby tissue such as lymph nodes; Stage II: the cancer has grown through the wall of the colon or rectum, into the outermost layers and may have invaded other nearby tissues, but has not yet spread to the nearby lymph nodes; Stage III: the cancer can be of any size, but has spread to 3 or fewer nearby lymph nodes, or has spread to 4 or more nodes but it has not spread to other parts of the body; Stage IV: the cancer has spread to distant organs such as the liver, lung, peritoneum or ovary.

The staging system for renal cell cancer is based on the degree of tumour spread beyond the kidney. Involvement of blood vessels may not be a poor prognostic sign if the tumour is otherwise confined to the substance of the kidney. Abnormal liver function test results may be due to a paraneoplastic syndrome that is reversible with tumour removal and do not necessarily represent metastatic disease. Except when computed tomography (CT) examination is equivocal or when iodinated contrast material is contraindicated, CT scanning is as good as or better than magnetic resonance imaging (MRI) for detecting renal masses.

The American Joint Committee on Cancer (AJCC) has designated staging by TNM classification.

TNM definitions

Primary tumour (T)
TX: Primary tumour cannot be assessed
T0: No evidence of primary tumour
T1: Tumour 7 cm or less in greatest dimension limited to the kidney
T2: Tumour more than 7 cm in greatest dimension limited to the kidney
   T3: Tumour extends into major veins or invades adrenal gland or perinephric tissues but not beyond Gerota's fascia
T3a: Tumour invades adrenal gland or perinephric tissues but not beyond Gerota's fascia
T3b: Tumour grossly extends into the renal vein(s) or vena cava below the diaphragm
T3c: Tumour grossly extends into the renal vein(s) or vena cava above the diaphragm
T4: Tumour invades beyond Gerota's fascia
Regional lymph nodes (N)
   NX: Regional lymph nodes cannot be assessed
   N0: No regional lymph node metastasis
   N1: Metastasis in a single regional lymph node
   N2: Metastasis in more than 1 regional lymph node
   [Note: Laterality does not affect the N classification.]
Distant metastasis (M)
   MX: Distant metastasis cannot be assessed
   M0: No distant metastasis
   M1: Distant metastasis
AJCC stage groupings
Stage I
   T1, N0, M0
Stage II
   T2, N0, M0
Stage III
   T1, N1, M0
   T2, N1, M0
   T3a, N0, M0
   T3a, N1, M0
   T3b, N0, M0
   T3b, N1, M0
   T3c, N0, M0
   T3c, N1, M0
Stage IV
   T4, N0, M0
   T4, N1, M0
   Any T, N2, M0
   Any T, Any N, M1

Early stages of tumour development shall be understood to refer to stages in tumour development in which the tumour has detectably spread no further than the lymph nodes local to the organ of the primary tumour. Typically, early stages will be considered to be stages I and II.

### Analytical Methods

The following analytical methods may be usefully used to determine the appropriate levels of the biomarkers in the present invention:

In very general terms in a complete blood cell a blood sample is drawn in a test tube containing an anticoagulant (EDTA, sometimes citrate) to stop it from clotting, and transported to a laboratory. Counting the cells may be performed manually. However, this process is generally automated by use of an automated analyzer. Automated blood counting machines include the Medonic M Series, Beckman Coulter LH series, Sysmex XE-2100, Siemens ADVIA 120 & 2120, the Abbott Cell-Dyn series, and the Mindray BC series. As a particular example the following assay may usefully be employed:

| ASSAY | METHOD/INSTRUMENT |
|---|---|
| COMPLETE BLOOD COUNT | BECKMAN Version: 7 |
| (includes Haemoglobin measurement) | COULTER GENS/LH750/LH780 |

The Coulter employs electronic counting and sizing of particles to quantitate and evaluate blood cells. Gen S/LH750/LH780 WBC Differential analysis and classification are based on simultaneous measuring of cell volume, high frequency conductivity and laser light scatter. Haemoglobin, released by hemolysis to either a stable cyanide containing pigment or oxyhaemoglobin-based hemachromagen, is measured by photometric absorbance.

An indirect solid phase enzyme immunoassay (ELISA) may be used for the quantitative measurement of ferritin in human serum or plasma. The assay described below by way of example is intended for in vitro diagnostic use only as an aid in the diagnosis and therapy control of iron deficiency:

20% of the human iron (total: 4-5 g) is reversely bound to ferritin as an intracellular storage protein. The remaining iron is bound to haemoglobin (60%) and myoglobin or enzymes (20%).

Ferritin has a molecular weight of 450 kDa and is located in various tissues, i.e. liver, spleen, and bone marrow or mucous of the bowels. Highly purified ferritin can develop red-brown crystals. Its 24 subunits form a hollow sphere to bind 4000 iron atoms connected to hydroxyphosphate residues. The iron-free protein is called apo-ferritin. The iron-loaded ferritin is the most important and most specific iron storage of the cells and of the whole organism. In case of iron-deficiency iron can be released quickly from ferritin and it is served in a bioavailable status.

Ferritin is found intracellular and in the blood stream. It is a reliable parameter to determine the iron concentration in the body. Serum ferritin concentrations remain constant during the biorhythm—in contrast to the alternating iron values. Ferritin values depend of the patient's age and sex. Regular losses of blood or blood donation decrease the ferritin values.

The determination of serum ferritin is an important parameter for the diagnosis and therapy control of an iron-deficiency. Negative iron-balance decreases the ferritin value. Ferritin contents below 12 ng/ml indicate a manifested iron-deficiency. During therapy with iron, ferritin values indicate the actual iron storage. Ferritin measurements are recommended for risk groups, like blood donors, pregnant women, hemodialysis patients and infants.

In some cases of iron-overloading serum ferritin values can exceed 500 ng/ml. Patients with hemochromatosis or secondary siderosis reveal elevated ferritin values. The whole clinical situation can only be evaluated by considering the entire diagnostic parameters.

### Indications:

iron-deficiency

iron-overloading

iron-deficiency anaemia

hemochromatosis

latent iron deficiency

liver diseases

risk groups

tumours

iron therapy

### Principle of the Test:

Anti-human-ferritin antibodies are bound to microwells. Ferritin, if present in diluted serum or plasma, binds to the respective antibody. Washing of the microwells removes unspecific serum and plasma components. Horseradish peroxidase (HRP) conjugated anti-human ferritin immunologically detects the bound patient ferritin forming a conjugate/ferritin/antibody complex. Washing of the microwells removes unbound conjugate. An enzyme substrate in the presence of bound conjugate hydrolyzes to form a blue colour. The addition of an acid stops the reaction forming a yellow end-product. The intensity of this yellow colour is measured photometrically at 450 nm. The intensity of colour is directly proportional to the concentration of ferritin present in the original sample.

### Test Procedure:

1. Prepare a sufficient number of microplate modules to accommodate controls and patient samples.
2. Pipette 25 μl of calibrators, controls and patient samples in duplicate into the wells.
3. Add 100 μl sample buffer to each well
4. Incubate for 30 minutes at room temperature (20-28° C). Discard the contents of the microwells and wash 3 times with 300 μl of wash solution
5. Dispense 100 μl of enzyme conjugate into each well.
6. Incubate for 15 minutes at room temperature.
7. Discard the contents of the microwells and wash 3 times with 300 μl of wash solution.
8. Dispense 100 μl of TMB substrate solution into each well.
9. Incubate for 15 minutes at room temperature.
10. Add 100 μl of stop solution to each well of the modules and incubate for 5 minutes at room temperature.
11. Read the optical density at 450 nm and calculate the results. Bi-chromatic measurement with a reference at 600-690 nm is recommended.
   The developed colour is stable for at least 30 minutes. Read optical densities during this time.

### Interpretation of Results:

### Quality Control

This test is only valid if the optical density at 450 nm for Positive Control (1) and Negative Control (2) as well as for the Calibrator A and F complies with the respective range indicated on the Quality Control Certificate enclosed to each test kit. If any of these criteria is not fulfilled, the results are invalid and the test should be repeated.

### Calculation of Results

For Ferritin ELISA a 4-Parameter-Fit with lin-log coordinates for optical density and concentration is the data reduction method of choice.

### Recommended Lin-Log Plot

First calculate the averaged optical densities for each calibrator well. Use lin-log graph paper and plot the averaged optical density of each calibrator versus the concentration. Draw the best fitting curve approximating the path of all calibrator points. The calibrator points may also be connected with straight line segments. The concentration of unknowns may then be estimated from the calibration curve by interpolation.

### EXAMPLES

### Example 1

### Study Design

A detailed description of the trial design has been published elsewhere (Amato et al,²²) and is also set out below. In brief, patients with advanced or metastatic clear cell renal cancer who had undergone prior nephrectomy, had a good or intermediate prognosis (MSKCC score 0-2), Karnofsky performance status >80% and life expectancy of >12 weeks were eligible.

MVA-5T4 (1×10⁹ TCID₅₀/ml) or placebo were administered by intra-muscular injection into the deltoid muscle at weeks 1, 3, 6, 9, 13, 17, 21, 25, 33, 41, 49, 57 and 65. During the course of the study, plasma samples were obtained from patients prior to treatment and following the 3^{rd} and 4^{th} MVA-5T4/placebo vaccinations (weeks 7 and 10 respectively) for assessment of MVA and 5T4-specific antibody responses. Furthermore, blood samples were obtained from 50 consenting and nominally healthy individuals, aged between 21 and 58 (mean=34) of whom 22 were male (44%); these served as controls for comparison against patients with cancer.

### Measurement of Antibody Responses

5T4 and MVA-specific antibody responses were determined as discussed above.

### Statistical Analysis

An immunological analysis set was defined comprising subjects with antibody response data at baseline (week 1) and at week 10 (after the 4^{th} MVA-5T4 vaccination). Within the MVA-5T4 group, the effect of quantitative 5T4 immune response (defined as the logarithm of the ratio of week 10 5T4 antibody level to the baseline level) on overall survival was assessed using a proportional hazards model. A similar analysis was performed looking at the association of MVA immune response with overall survival.

The immune response surrogate (IRS) was constructed by finding all baseline hematological, immunological, demographic and cancer characterizing variables that predicted quantitative 5T4 immune response with a significance of P less than 0.20 within a general linear model. These variables were then used as the initial model in a backwards elimination procedure which only retained those associated with a P-value of less than 0.05, all models being adjusted for standard of care (IL-2, IFN-α or Sunitinib). The variables retained in the model were used to construct a predictor of immune response (the IRS) which was calculated for all subjects in the immune response set. The IRS was then used in a proportional hazards model to assess treatment benefit on overall survival.

All survival analyses were performed three times with retrospective censoring at 12, 18 and 24 months from randomization respectively in order to establish the robustness of the conclusions, given uncertainty over the time period over which MVA-5T4 shows benefit. All proportional hazards models were stratified by standard of care with separate treatment effects for each standard of care.

The IRS derived from the TRIST study was also applied to a historic dataset derived from 9 separate phase I and II studies of MVA-5T4 in patients with renal, colorectal and prostate cancer (7-15). In all 9 studies, antibody responses against 5T4 and MVA were assessed in the same manner as described for this study. Likewise, an immunological analysis set was defined comprising subjects with antibody response data at baseline and after the 4^{th} MVA-5T4 vaccination. This immunological analysis set contained antibody data from 52 patients with renal cancer, 32 with colorectal cancer and 24 with prostate cancer.

### Results

### 5T4 and MVA Antibody Levels

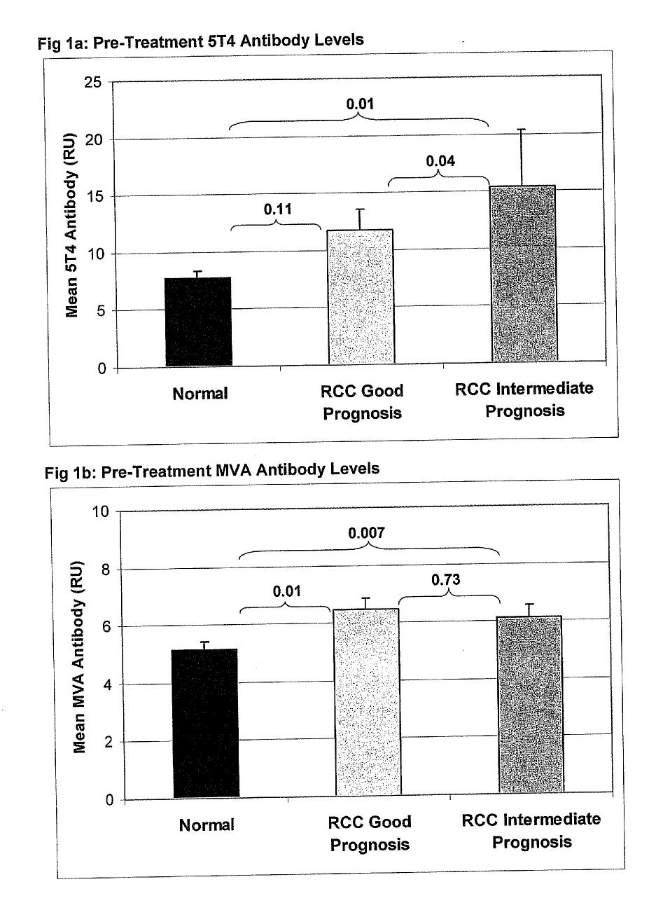

During the course of the TRIST study, antibody responses against the 5T4 tumour antigen and the MVA viral vector were determined at baseline (pre-treatment) and at weeks 7 and 10 (following the 3^{rd} and 4^{th} MVA-5T4/placebo vaccinations). Prior to treatment with MVA-5T4 or placebo, positive 5T4-specific antibody responses were detected in 81 (23%) and 99 (27%) patients respectively; positive MVA-specific antibody responses were detected in 98 (27%) and 87 (24%) patients respectively (data not shown). The magnitude of pre-treatment 5T4 and MVA antibody levels in renal cancer patients were compared to those found in plasma samples recovered from 50 nominally healthy donors (FIGS. 1a and 1b respectively). Pre-treatment 5T4 and MVA antibody levels were elevated in RCC patients compared to healthy donors (P=0.036 and 0.008 respectively; data not shown). Furthermore, stratification by MSKCC score showed that RCC patients classified as good prognosis (MSKCC=0) or intermediate prognosis (MSKCC=1-2) had elevated 5T4 antibody levels compared to healthy donors (FIGS. 1a; P=0.11 and P=0.009 respectively). Interestingly, patients classified as intermediate prognosis had higher 5T4 antibody levels compared to good prognosis patients (P=0.04). MVA antibody levels were also higher in good and intermediate prognosis RCC patients compared to healthy donors (FIG. 1b; P=0.01 and 0.007 respectively). However, unlike 5T4 there was no difference in pre-treatment MVA antibody levels in good prognosis patients compared to intermediate prognosis patients (P=0.73).

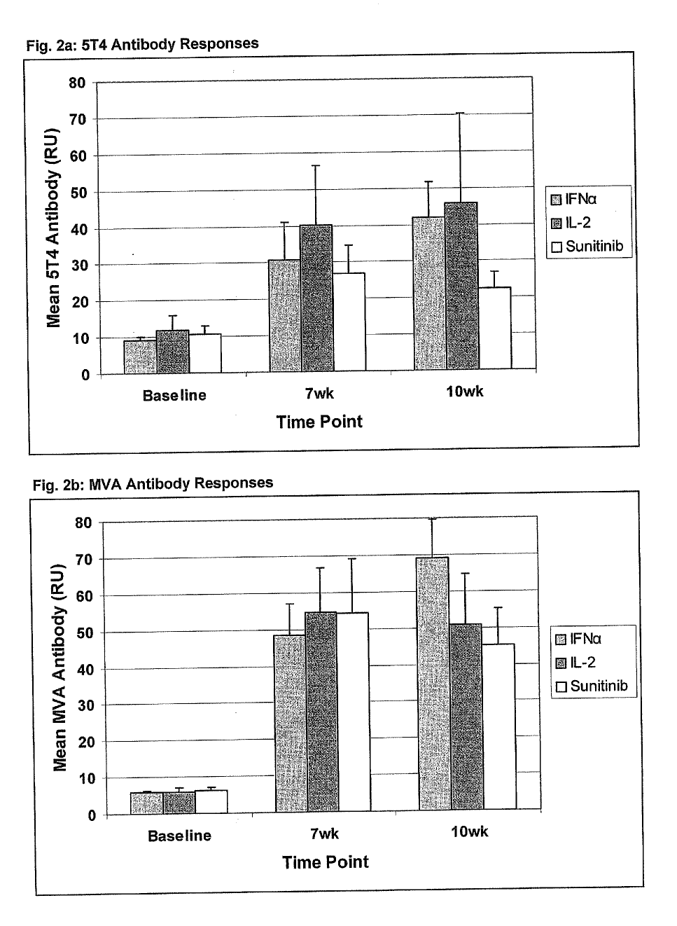

The percentage of patients who were classified as having mounted a positive 5T4-specific antibody response, relative to pre-treatment levels, following the 3^{rd} and/or 4^{th} vaccination with MVA-5T4 or placebo was 56% and 6% respectively. An analysis of the magnitude of 5T4 (FIG. 2a) and MVA (FIG. 2b) antibody responses was undertaken in patients treated with MVA-5T4 and classified as 5T4 sero-converters. In this sub-set of patients, the magnitude of antibody responses was similar at each time point and for each SOC, with the only significant difference in the magnitude of 5T4 or MVA antibody responses being at week 10 in patients receiving IFN-a compared to sunitinib (Mann-Whitney; both P<0.01).

Association between Immunological Response and Overall Survival

The immunological analysis set contained 590 individuals, 288 in the MVA-5T4 treated group and 302 in the placebo group. Table 1 shows the number of events in each group under each of the three censoring regimens. A total of 143 randomized individuals were excluded from the immunological analysis set: 140 because of missing immunological data, 2 were randomized in error and 1 had an outlying high baseline 5T4 antibody level. In order to have a non-missing week 10 assessment, the subject must have survived at least to that assessment. Fifty subjects (25 in each of the two treatment groups) either died (20 in each of the two groups) or were lost to follow-up (5 in each of the two groups) on or before day 70.

The quantitative 5T4 immune response was positively associated with longer survival (hazard ratio of approximately 0.78 and P<0.05) at all three censoring time points (12, 18 and 24 months; Table 2). A hazard ratio less than unity indicates that the hazard decreases with increasing immune response. In contrast, the quantitative MVA immune response was not significantly associated with survival (hazard ratio -1 and P>0.10) at all three censoring time points.

### Obtaining a Surrogate for Quantitative 5T4 Immune Response

The baseline variables which individually correlated with quantitative 5T4 immune response with a P-value of less than 0.10 after adjustment for standard of care are listed in Table 3.

The backwards elimination algorithm applied to the variables in Table 3 resulted in three factors remaining in the model for quantitative 5T4 immune response, after adjustment for standard of care: the logarithm of the baseline 5T4 antibody level (P=0.0004), haemoglobin level (P=0.0071) and haematocrit (P=0.028). The regression equation for quantitative 5T4 immune response has two components: a term depending on standard of care, and the IRS with formula:

$$IRS = - 0.292 \times ( natural log of baseline 5 T4 antibody level ) + 0.0224 \times ( haemoglobin level in g \text{/} L ) + - 6.16 \times ( haematocrit as a fraction )$$

It is noteworthy that the sign associated with haematocrit is negative in the IRS despite being positive when the model just contained haematocrit. The form of the IRS is indicating that, for a given level of haemoglobin, response is negatively associated with haematocrit.

### Using the Immune Response Surrogate to Model Treatment Benefit

The IRS and its interaction with treatment were included in a proportional hazards model of overall survival stratified by standard of care and including separate treatment effects for each standard of care. Table 4 shows the hazard ratios associated with the effect of IRS in the placebo group and in the MVA-5T4 group, together with their quotient. The hazard ratios being less than unity in the individual treatment arms shows that the IRS is prognostic within each treatment group: the higher the value of IRS, the lower the hazard (and the longer the survival).

Treatment benefit is assessed by dividing the hazard ratio for the IRS in the MVA-5T4 group by the hazard ratio in the placebo group: a result less than unity means that the higher the IRS, the lower the hazard ratio of MVA-5T4 against placebo. Under all three censoring regimens, the IRS is a statistically significant predictor of treatment benefit.

Given the relationship between the immune response surrogate and treatment benefit, we undertook further exploratory analyses using components of the IRS to identify large sub-sets of the TRIST population who may have received significant clinical benefit from MVA-5T4. An exemplary sub-set of patients who were in both the top 50% for baseline haemoglobin-haematocrit ratio and bottom 50% for baseline 5T4 antibody was constructed. This sub-set of patients showed a hazard ratio of 0.52 in favor of MVA-5T4 (146 subjects, P=0.011). Relaxing the inclusion criteria to the top and bottom 60% respectively yielded a hazard ratio of 0.56 (211 subjects, P=0.0063).

### Application of the IRS to the Phase II Studies

Following the derivation of the immune response surrogate using the TRIST dataset, the IRS was applied to historical data from previous phase I and II studies of MVA-5T4 in patients with renal, colorectal and prostate cancer. From the phase I and II studies, 108 evaluable patients contributed to the immunological and survival dataset. When the IRS was applied to these data, it was positively associated with quantitative 5T4 antibody response (P<0.0001, adjusted for indication and study; P=0.017, renal cancer subjects only, adjusted for study) and with overall survival (P=0.0034, stratified by indication and study; P=0.0023, renal cancer subjects only, stratified for study). In the renal cancer subjects, the hazard ratio was 0.076 (0.95 CI: [0.014, 0.398]) which is consistent with the hazard ratio of approximately 0.2 seen in the MVA-5T4 column of Table 4.

TABLE 1
Censoring Regimens. The table presents numbers of subjects lost to
follow-up (LTFU) before cut-off date, censored (at earlier of cut-off
date and end of study) and died before cut-off dates for three censoring
regimens (retrospectively censored at cut-off dates 12, 18 and 24
months after randomization).

| | Placebo group | | | | MVA-5T4 group | | | |
|---|---|---|---|---|---|---|---|---|
| Censoring | Numbers of Subjects | | | | | | | |
| Cut-off | Total | LTFU | Censored | Died | Total | LTFU | Censored | Died |
| 12 months | 302 | 2 | 220 | 80 | 288 | 1 | 201 | 86 |
| 18 months | 302 | 5 | 173 | 124 | 288 | 4 | 157 | 127 |
| 24 months | 302 | 8 | 139 | 155 | 288 | 9 | 136 | 143 |

TABLE 2
The Effect of Antibody Response on Survival. Response is defined
as the natural logarithm of the ratio of antibody level at
week 10 to that at baseline. A proportional hazards model for
overall survival was used stratified by standard of care. The
hazard ratios correspond to a doubling of the ratio of the
antibody level at week 10 compared to baseline.

| Censoring | Effect of MVA Response | Effect of 5T4 Response |
|---|---|---|
| Cut-off | Hazard Ratio [0.95 Confidence Interval] (P-value) | |
| 12 months | 1.154 [0.940, 1.416] (0.17) | 0.789 [0.634, 0.983] (0.035) |
| 18 months | 1.140 [0.964, 1.349] (0.13) | 0.783 [0.656, 0.936] (0.0071) |
| 24 months | 1.060 [0.907, 1.239] (0.46) | 0.789 [0.666, 0.934] (0.0059) |

TABLE 3
Univariate Predictors of Quantitative 5T4 Antibody Response.
The direction of effect is positive if an increase in the
baseline variable is associated with an increase in the immune
response and negative if an increase in the baseline variable
is associated with a decrease in the immune response. Only
those variables which are associated with a P-value of less
than 0.1 are included in the table.

| | Direction of | |
|---|---|---|
| Baseline Variable | Effect | P-value |
| Haemoglobin Level | Positive | 0.0031 |
| Baseline 5T4 Antibody Level Below LLQ^{†} | Positive | 0.0081 |
| Haematocrit | Positive | 0.026 |
| Baseline VEGF Level Below LLQ^{†} | Positive | 0.042 |
| Baseline MVA Antibody Level Below LLQ^{†} | Positive | 0.050 |
| Bilirubin Concentration* | Positive | 0.058 |
| Red Blood Cell Count | Positive | 0.069 |
| Age | Negative | 0.099 |
| Urea Concentration | Negative | 0.079 |
| Baseline VEGF Level* | Negative | 0.052 |
| Alkaline Phosphatase Level* | Negative | 0.015 |
| Platelet Count* | Negative | 0.012 |
| Baseline 5T4 Antibody Level* | Negative | <0.0001 |

[Key:
*The natural logarithm of these baseline variables was taken after inspection of the distributions;
^{†}Indicator for below the lower limit of quantitation.]

TABLE 4
The Effect of the Immune Response Surrogate (IRS) on Survival.
A proportional hazards model for overall survival was used, stratified
by standard of care. The hazard ratios correspond to a doubling
of predicted quantitative 5T4 antibody response.

| | Prognostic Effect of | Prognostic Effect of | Treatment Benefit |
|---|---|---|---|
| Censoring | IRS in Placebo Group | IRS in MVA-5T4 Group | Associated with IRS |
| Cut-off | Hazard Ratio [0.95 Confidence Interval] (P-value) | | |
| 12 months | 0.555 [0.322, 0.956] | 0.153 [0.085, 0.273] | 0.275 [0.124, 0.61] |
| | (0.0339) | (<0.0001) | (0.0015) |
| 18 months | 0.62 [0.394, 0.975] | 0.233 [0.14, 0.387] | 0.375 [0.19, 0.741] |
| | (0.0387) | (<0.0001) | (0.0048) |
| 24 months | 0.704 [0.463, 1.068] | 0.251 [0.154, 0.41] | 0.357 [0.188, 0.68] |
| | (0.0991) | (<0.0001) | (0.0017) |

### Example 2

### Anaemia-Associated IRS Factors include Iron Status of Patients via Analysis of Plasma-Ferritin Levels

Two of the baseline factors contributing to the IRS, namely haemoglobin and haematocrit, are indicators of anaemia. Anaemia can be caused and is manifest in many ways, but one of the key etiological factors is iron deficiency. Therefore assessment of iron status may provide a more sensitive predictor of performance than haemoglobin and haematocrit alone. As such, baseline (pre-vaccination) plasma samples from patients who have participated in TroVax clinical trials are assessed for ferritin levels using a commercially available ELISA kit (Human Ferritin ELISA kit DE7750 from Demeditec Diagnostics GmbH, Germany) for the measurement of plasma-ferritin. This ferritin level is then be assessed for contribution to the IRS.

### Example 3

### Study Details—TRIST

The study was termed TRIST: TroVax® Renal Immunotherapy Survival Trial. An international Phase III, randomized, double blind, placebo controlled, parallel group study to investigate whether TroVax® added to first-line standard of care therapy, prolongs the survival of patients with locally advanced or metastatic renal clear cell adenocarcinoma.

The primary purpose of this trial is to demonstrate the effect of TroVax® on survival in patients with locally advanced or metastatic renal clear cell adenocarcinomas. Clear cell adenocarcinomas of the kidney uniformly express 5T4 at high concentrations (80-90% of tumours examined) and are therefore an obvious candidate for treatment with a 5T4 vaccine.

Reported median survival times for this indication vary between studies but are generally in the range of 6 to 18 months depending on patient's status at entry and to a lesser extent on treatment. Novel forms of treatment are urgently needed.

This study will assess the impact on survival of adding TroVax® to the first-line standard of care for renal cancer. The current standard of care varies between countries and institutions and is influenced by the patient's status, the national regulatory status of different treatments and local reimbursement considerations. Commonly accepted standards of care for renal cancer include IL-2, IFNα, or a receptor tyrosine kinase inhibitor such as sunitinib. The use and availability of these treatments varies geographically.

High dose IL-2, although approved for the treatment of renal cancer is not included in this study as the high incidence of serious adverse events and need for intensive care limit its application and would complicate the safety evaluation of TroVax®.

The rationale for the potential concurrent use of IL-2 is that this compound is believed to act as an adjuvant. IL-2 is currently one of the standards of care regimens for the first line treatment of advanced and metastatic renal cancer. The dose schedule of IL-2 chosen is well recognised by the oncology community and has been validated in large scale phase III clinical trials. Over 30 patients treated with a combination of TroVax® and IL-2 (high dose intravenous or low dose subcutaneous regimens) have been assessed in phase II studies in patients with renal cancer. The combination was well tolerated. Compared with the historical adverse event profile of IL-2 alone the only additional adverse events reported were minor local reactions at the site of TroVax® injection and mild transient pyrexia. Humoral and/or cellular immune responses to 5T4 were induced in almost all patients and objective responses by RECIST have been reported.

Although it is not clear whether the biologic effects of IFNα occur entirely or in part via immunostimulation, there is evidence to show that it does have a modest clinical effect in renal cancer patients with an objective response rate of approximately 7.5-15%. Studies to determine whether IFNα increases survival in patients with renal cancer have produced inconsistent results. Given the immunological mechanism of action of IFNα, it is reasonable to evaluate the effect of TroVax® on survival in patients receiving this common standard of care. An ongoing study has not indicated any untoward safety impact resulting from co-administration of IFNα and TroVax®.

Phase II studies including over 20 patients treated with a combination of TroVax® and IFNα (three times weekly subcutaneous regimen) are ongoing in patients with renal cancer. Developing data indicate the combination to be well tolerated. Compared with the historical adverse event profile of IFNα alone the only additional adverse events reported are minor local reactions at the site of TroVax® injection and mild transient pyrexia. The expected humoral and/or cellular immune responses to 5T4 will be confirmed. Interim study reports will be available for review by regulatory authorities, IRB/Ethics Committees and investigators as part of the approval process of this study.

Recently developed oral kinase inhibitors, such as sorafenib and sunitinib, are becoming increasingly important in the management of advanced or metastatic renal cell carcinoma. Safety and immunology data necessary to support coadministration of sorafenib and TroVax® are not available. In view of this and the higher overall response rate reported with sunitinib the latter will be included in this study as an example of a kinase inhibitor used in the treatment of renal cancer.

Therefore, in regions where this treatment is approved, sunitinib may be used as the standard of care alongside TroVax®/placebo in this study. A phase II study of patients treated with a combination of TroVax® and sunitinib (50 mg oral dose taken once daily, on a schedule of 4 weeks on treatment followed by 2 weeks off) is ongoing in patients with renal cancer. Developing data indicate the combination to be well tolerated. Compared with the reported data on sunitinib alone the only additional adverse events reported are minor local reactions at the site of TroVax® injection and mild transient pyrexia. The expected humoral and/or cellular immunes response to 5T4 are to be confirmed. An interim study report will be available for review by regulatory authorities, IRB/Ethics Committees and investigators as part of the approval process of this study.

A cancer vaccine is intended to prolong survival by inducing an immune response to a tumour associated antigen. Preclinical models indicate that cancer vaccines may delay tumour growth and reduce the number of new metastases. It is not yet known whether a cancer vaccine must produce a high objective tumour response rate (by RECIST) in order to have clinically useful effect on prolonging survival. This will only be determined by a randomised survival study in patients receiving adequate vaccination to reliably induce an efficacious immune response. To date, both disease stabilization and late tumour responses have been reported with various cancer vaccines.

The maximum immunological response to TroVax® dose not usually occur until the patient has received a minimum of three injections and it is not yet established whether continuing TroVax® despite early progression will confer therapeutic benefit. Therefore, in this study, if tumour progression is observed but the patient is tolerating TroVax®/placebo and their performance status remains at a Karnofsky score >60%, they should be requested to continue on study receiving TroVax®/placebo until they have received a minimum of eight injections of the study preparation. Continuation on study beyond this point to receive all TroVax®/placebo injections is permitted for such patients but is at the discretion of the investigator or patient.

A randomized, parallel group, double blind design is standard in phase III efficacy studies. Interim statistical analyses conducted by an independent Data Safety Monitoring Board according to a pre-specified charter will be based on these interim analyses of safety and efficacy. The DSMB may recommend continuation of the study, stopping the study or stopping enrolment of patients of a specific treatment cell. The DSMB will also assess whether the frequency of events in the control arm matches the predictions used to determine the sample size of the study and may recommend changes to the number of events (deaths) triggering the final analysis.

TroVax® is a vaccine against a tumour-associated antigen. The assessment of such tumour vaccines for patients with solid tumours is complicated by a number of factors which influence the definition of the objectives, the route to achieving the objectives and the ongoing management of patients in the study.

Special features of tumour vaccines that are relevant to the objectives are listed below:
Vaccine-mediated immunotherapy requires repeated administration and time for the patient to develop an immune response to the vaccine antigen. In previous phase II studies it was shown that at least three administrations of TroVax® were required to generate a significant immune response. This means that patients who are removed from the study medication before receiving three injections of TroVax® due to death or rapidly progressive renal cancer, do not allow assessment of the potential of a TroVax®-induced immune response to provide benefit to patients treated for a longer period.
Cancer vaccines such as TroVax® may exert beneficial effects in delaying tumour growth and metastasis that do not manifest as RECIST responses but may prolong survival. This has implications for the management of patients because patients with a RECIST classification of progressive disease may still benefit from continuing with TroVax.
It is not yet known whether tumour shrinkage predicts survival advantage. This means that the definitive efficacy endpoint is survival.

Objectives

Primary Efficacy Objective

To assess whether the addition of TroVax® to first line standard of care, will prolong survival of patients with locally advanced or metastatic clear cell renal adenocarcinoma when compared to placebo.

Analysis will occur after a predetermined number of deaths have occurred necessary to trigger the primary endpoint analysis or when specified by an independent Data Safety Monitoring Board based on analyses of interim data.

The analysis will be based on the Intent to Treat (ITT) population, composed of all patients.

Primary Safety Objective

To assess whether the addition of TroVax® to first line standard of care alters the profile of serious and non-serious adverse events, when compared to placebo, in patients with locally advanced or metastatic clear cell renal adenocarcinoma. This will be assessed in the Intent to Treat (ITT) population.

Secondary Efficacy Objectives
To compare the proportion of patients with progression free survival at 26 weeks (+/−1 week) in the TroVax® versus placebo arms based on radiological data. Data will be analysed using the ITT population and adjudicated (blinded peer review) baseline and week 26 radiological data.
To compare the tumour response rates, time to response and duration of response between patients treated with TroVax® versus placebo. This will be analysed in the Intent to Treat (ITT) population.

To assess whether the addition of a minimum of three doses of TroVax® to first line standard of care, will prolong survival of patients with locally advanced or metastatic clear cell renal adenocarcinoma when compared to placebo. This will be an exploratory analysis in the Modified Intent to Treat (MITT) population.

To assess whether TroVax® has an impact on the quality of life as measured by QLQ30 and EuroQOL questionnaires when compared to placebo. This will be analysed in the Intent to Treat (ITT) population.

Endpoints

Primary Efficacy Endpoint

The survival event rate ratio in the TroVax® arm versus the placebo in the Intent to Treat (ITT) population based on the log of the hazard ratio derived from the Cox Proportional Hazards regression model. A frequentist monitoring approach will be used for evaluating the event ratio.

The key objective of this study is to determine whether TroVax® is able to prolong survival in patients receiving first line standard of care.

Analysis is triggered by a predetermined number of deaths in the study population or when specified by an independent Data Safety Monitoring Board based on analyses of interim data.

Primary Safety Endpoints

The number of adverse events (serious and non-serious) in the Intent to Treat population in the TroVax® versus the placebo arm.

The laboratory variables (complete blood count and chemistry panel) in the Intent to Treat (ITT) population in the TroVax® versus the placebo arm.

Secondary Efficacy Endpoints

The proportion of patients in the TroVax® versus placebo arms in the Intent to Treat (ITT) population with progression free survival at 26 weeks based on a comparison of baseline and week 26 (+/−1 week) radiological data and using RECIST criteria. Data will be adjudicated (blinded peer review).

Tumour response rates according to the investigator's reported interpretation of the radiological reports based on RECIST criteria observed in the Intent to Treat (ITT) population.

The survival event rate ratio in the TroVax® arm versus the placebo in the Modified Intent to Treat (MITT) population based on the log of the hazard ratio derived from the Cox Proportional Hazards regression model. A frequentist monitoring approach will be used for evaluating the event ratio.

The quality of Life score for TroVax® versus placebo as measured by QLQ30 and EuroQOL questionnaires in the Intent to Treat (ITT) and Per Protocol populations.

Immunology Endpoint

Anti-5T4 antibody levels (additional measures of immune response including specific measures of cellular response will be investigated at some centres. Each will be the subject of a separate related protocol and informed consent for specific study sites and will be conditional upon regulatory and IRB/ethics committee approval before implementation.)

Metastatic renal cancer has a poor prognosis. The median survival overall has been reported to be as low as 6 months and five year survival is <5% . Conventional systemic cytotoxic chemotherapeutic agents and hormonal therapies have little impact on survival and response rates are usually <10%. The wide variations in the natural history of the disease and spontaneous regression rates of up to 6% have led to the investigation of immune mechanisms as a factor influencing responses and outcomes. Biological and immunologic therapies have demonstrated the best response rates with some impact on overall survival. However the management of metastatic renal cancer remains a therapeutic challenge.

Interferon alpha (IFNα) has demonstrated response rates of 8-26% with median survivals of 13 months. Interleukin-2 (IL-2) induces responses in 7-23% of patients with a median survival of 12 months. The benefit of biologic agents has been confirmed by randomised controlled trials, which have shown modest survival benefits with IFNα compared with medroxprogesterone or vinblastine. Motzer (2004) “Prognostic factors for survival of patients with stage IV renal cell carcinoma: Memorial Sloan-Kettering Cancer Center experience.” Clin Cancer Res 10 (18 Pt 2): 6302S-3S, in a retrospective analysis of 670 patients in 24 trials of systemic chemotherapy or cytokine therapies, demonstrated longer survival times with cytokine therapy. In the group who were long term survivors, 70% were in trials that involved IFNα □and /or IL-2 and 30% had been treated with hormonal or cytotoxic agents.

The initial studies with IL-2 used protocols based on the principles of chemotherapy, using maximum tolerated doses. This was associated with significant renal, cardiac, pulmonary and haemodynamic toxicity, often requiring admission to intensive care wards and limiting utility to a selected subsection of the patient group. Subsequent studies of IL-2 have demonstrated similar efficacy, but with significantly less toxicity, using lower doses administered subcutaneously on an outpatient basis. In a study, comparing high and low-dose IL-2, there was a higher response rate with high dose treatment but this did not translate into survival benefit.

Negrier et al. “Recombinant human interleukin-2, recombinant human interferon alfa-2a, or both, in metastatic renal-cell carcinoma. Groupe Francaise d′Immunotherapie. N Engl J Med 1998; 338; 1272-8 assessed the use of these biologic agents as single agent therapy or combination therapy. They demonstrated response rates of 6.5%, 7.5% and 18.6% for IFNα, IL-2 or the combination, respectively. Although there was a difference in progression free survival, this did not translate into a survival advantage. The rationale for the combination of these agents is that, in vitro, IFNα enhances cell membrane expression of major histocompatibility antigens to which IL-2 activated T-cells can respond.

There is well-documented evidence to suggest that selection and prognostic factors significantly influence outcomes and responses to cytokine therapies. Motzer has assessed the prognostic value of a number of variables in patients with advanced or metatstic renal cell carcinoma. In these patients, low Karnofsky performance status, low haemoglobin level and high corrected serum calcium level indicated a poor prognosis. The median time to death in patients with zero risk factors was 22 months. The median survival in patients with one of these risk factors was 11.9 months and patients with 2-3 risk factors had a median survival of 5.4 months.

Two new drugs have recently been developed for the management of renal cancer: sunitinib and sorafenib. Both function by inhibiting multiple receptor kinases. Overall (complete and partial) response rates reported with sunitinib are substantially higher (25.5-36.5%) than reported with sorafenib (2%) though information on time to tumour progression and survival is still maturing.

Safety and immunology data necessary to support coadministration of sorafenib and TroVax® are not available. In view of this and the higher overall response rate reported with sunitinib the latter will be included in this study as an example of a receptor tyrosine kinase inhibitor used in the treatment of renal cancer.

Sunitinib malate is a small molecule that inhibits multiple receptor tyrosine kinase (RTKs), some of which are implicated in tumour growth, pathologic angiogenesis, and metastatic progression of cancer. Sunitinib was evaluated for its inhibitory activity against a variety of kinases (>80 kinases) and was identified as an inhibitor of platelet-derived growth factor receptors (PDGFRα and PDGFR(3), vascular endothelial growth factor receptors (VEGFR1, VEGFR2 and VEGFR3), stem cell factor receptor (KIT), Fms-like tyrosine kinase-3 (FLT3), colony stimulating factor receptor Type 1 (CSF-1 R), and the glial cell-line derived neurotrophic factor receptor (RET). Sunitinib inhibition of the activity of these receptor tyrosine kinase (RTKs) has been demonstrated in biochemical and cellular assays, and inhibition of function has been demonstrated in cell proliferation assays. The primary metabolite exhibits similar potency to sunitinib when compared in biochemical and cellular assays.

The use of single agent sunitinib in the treatment of cytokine-refractory MRCC was investigated in two single-arm, multi-centre studies. All patients enrolled into these studies experienced failure of prior cytokine-based therapy. The primary endpoint for both studies was overall response rate (ORR). Duration of response (DR) was also evaluated.

One hundred and six patients were enrolled into Study 1, and 63 patients were enrolled into Study 2. Across the two studies, 95% of the pooled population of patients had at least some component of clear-cell histology. Patients received 50 mg sunitinib in cycles with 4 weeks on and 2 weeks off. Therapy was continued until the patients met withdrawal criteria or had progressive disease. There were 27 PRs in Study 1 as assessed by a core radiology laboratory for an ORR of 25.5% (95% CI 17.5, 34.9). There were 23 PRs in Study 2 as assessed by the investigators for an ORR of 36.5% (95% CI 24.7-49.6). The majority (>90%) of objective disease responses were observed during the first four cycles; the latest reported response was observed in cycle 10. DR data from Study 1 is premature as only 4 of 27 patients (15%) responding to treatment had experienced disease progression. At the time of the data cut-off, Study 1 was ongoing with 44 of 106 patients (41.5%) continuing treatment, and 11 of the 63 patients (17.5%) enrolled on Study 2 continued to receive sunitinib on continuation protocols .

As of March 2006 no data are available to determine whether sunitinib (or sorafenib) prolongs survival in patients with renal cancer.

Despite recent development of the kinase inhibitors, stage IV renal cell carcinoma is an area of high unmet medical need. The use of vaccines in this area is novel but capitalises on the accepted opinion that immunologic mechanisms may have a part to play in the treatment of this disease.

Primary efficacy objective

To assess whether the addition of TroVax® to first line standard of care, will prolong survival of patients with locally advanced or metastatic clear cell renal adenocarcinoma when compared to placebo. This will be assessed in the Intent to Treat (ITT) population.

Primary safety objective

To assess whether the addition of TroVax® to first line standard of care alters the profile of serious and non-serious adverse events, when compared to placebo, in patients with locally advanced or metastatic clear cell renal adenocarcinoma. This will be assessed in the Intent to Treat (ITT) population.

Secondary efficacy objectives

To compare the proportion of patients with progression free survival at 26 weeks in the TroVax® versus placebo arms. This will be assessed in the Intent to Treat,(ITT) population.

To compare the tumour response rates, time to response and duration of response between patients treated with TroVax® versus placebo. This will be analysed in the Intent to Treat (ITT) population.

To assess whether the addition of a minimum of three doses of TroVax® to first line standard of care will prolong survival of patients with locally advanced or metastatic clear cell renal adenocarcinoma when compared to placebo. This will be an exploratory analysis in the Modified Intent to Treat (MITT) population.

To assess whether TroVax® has an impact on the quality of life as measured by QLQ30 and EuroQOL questionnaires when compared to placebo. This will be analysed in the Intent to Treat (ITT) population.

Study Endpoints

Primary Efficacy Endpoint

The survival event rate ratio in the TroVax® arm versus the placebo in the Intent to Treat (ITT) population based on the log of the hazard ratio derived from the Cox Proportional Hazards regression model. A frequentist monitoring approach will be used for evaluating the event ratio.

Primary Safety Endpoints

The number of adverse events (serious and non-serious) in the Intent to Treat population in the TroVax® versus the placebo arm.

The laboratory variables (complete blood count and chemistry panel) in the Intent to Treat (ITT) population in the TroVax® versus the placebo arm.

Secondary Efficacy Endpoints

The proportion of patients in the TroVax® versus placebo arms in the Intent to Treat (ITT) population with progression free survival at 26 weeks based on a comparison of baseline and week 26 (+/−1 week) radiological data and using RECIST criteria. Data will be adjudicated (blinded peer review).

Tumour response rates according to the investigator's reported interpretation of the radiological reports based on RECIST criteria observed in the Intent to Treat (ITT) population.

The survival event rate ratio in the TroVax® arm versus the placebo in the Modified Intent to Treat (MITT) population based on the log of the hazard ratio derived from the Cox Proportional Hazards regression model. A frequentist monitoring approach will be used for evaluating the event ratio.

The Quality of Life score for TroVax® versus placebo as measured by QLQ30 and EuroQOL questionnaires in the Intent to Treat (ITT) and Per Protocol populations.

Immunology Endpoint

Anti-5T4 antibody levels (additional measures of immune response including specific measures of cellular response will be investigated at some centers. Each will be the subject of a separate related protocol and informed consent for specific study sites and will be conditional upon regulatory and IRB/ethics committee approval before implementation.)

Study Population

Patients of any ethnic group with histologically proven clear cell renal adenocarcinoma who have had their primary tumour surgically removed and require treatment for locally advanced or metastatic disease. The intent is to include 700 patients split equally between the TroVax® and placebo arms.

Study Design

This is an international, randomized, double blind, placebo controlled, parallel group study to investigate whether a minimum of three doses of TroVax® added to first-line standard of care therapy, prolongs the survival of patients with locally advanced or metastatic renal clear cell adenocarcinoma.

The primary endpoint is survival. The study is designed to be pragmatic, limiting additional study related investigations to a minimum. Protocol mandated scans and X-rays are limited to two time points (baseline and week 26) to permit comparison of the percentage of patients with progressive disease at 6 months as a secondary efficacy endpoint. Six months was selected based on review of published literature indicating that progressive disease was commonly observed by 26 weeks in patients with renal cancer. Endpoints such as tumour response by RECIST are considered of secondary importance to survival and will be determined by radiological examinations ordered at the discretion of the investigator based on the clinical status of the patient and will be based the interpretation of the patient's care-team (investigator and local radiologist).

Study enrolment will only commence at each centre once ethics and regulatory approval have been obtained from the relevant authorities.

After signing the study informed consent form and meeting the baseline enrolment criteria patients will be assigned by the investigator (their physician) to one of the following defined first-line standard of care regimens based on what is best for the patient and consistent with local practice:
1. subcutaneous low dose IL-2
2. interferon alpha (excluding pegylated IFNalpha)
3. sunitinib

Only after the standard of care therapy has been decided should the investigator telephone the Interactive Voice Recognition Service (IVRS). Randomization to TroVax® or placebo will be stratified based on the standard of care chosen by the investigator, study prognostic indicators (Motzer score) and geography.

TroVax® is administered at a dose of 1×10⁹TCID50/ml in 1 ml by injection into the deltoid muscle of the upper arm at regular intervals up to 8 weeks apart up to a maximum of 13 doses.

An independent Data Safety Monitoring Board will be responsible for preparing the formal monitoring rules for this study. This parallel-designed study contains a series of planned interim assessments for futility, and to ensure that the planning elements relative to attrition and the primary endpoint remain consistent. A frequentist monitoring approach will be used for evaluating the event rate ratio to ensure that the assumptions are accurate and the sample size continues to be appropriate for assessing superiority. The DSMB may recommend changes to the enrollment target if pretrial assumptions prove inaccurate. These DSMB reviews will be conducted confidentially. Data analysis will not be shared with the sponsor, investigators or any other participant in the study.

Study Design

Type of Study

This is an international, randomised, double blind, placebo controlled, parallel group study designed to assess whether, when added to first-line standard of care, TroVax® prolongs survival in patients with locally advanced or metastatic renal carcinoma.

The primary endpoint is survival. The study is designed to be pragmatic, limiting additional study related investigations to a minimum. Protocol mandated scans and X-rays are limited to two time points (baseline and week 26) to permit comparison of the percentage of patients with progressive disease at 6 months as a secondary efficacy endpoint. Six months was selected based on review of published literature indicating that progressive disease was commonly observed by 26 weeks in patients with renal cancer. Endpoints such as tumour response by RECIST are considered of secondary importance to survival and will be determined by radiological examinations ordered at the discretion of the investigator based on the clinical status of the patient and will be based the interpretation of the patient's care-team (investigator and local radiologist).

Study enrolment of 700 patients will only commence once ethics and regulatory approval has been obtained from the relevant authorities.

After signing the study informed consent form and meeting the baseline enrolment criteria patients will be assigned by the investigator (their physician) to one of the following defined standard of care regimens based on what is best for the patient and consistent with local practice:
1. subcutaneous low dose IL-2
2. interferon-a (excluding pegylated IFNα)
3. sunitinib

Only after the standard of care therapy has been decided should the investigator telephone the Interactive Voice Randomisation Service (IVRS). Randomisation to TroVax® or placebo will be stratified based on the standard of care chosen by the investigator, the study site and prognostic indicators.

An independent Data Safety Monitoring Board will periodically review emerging data. These reviews will be conducted confidentially. Data analysis will not be shared with the sponsor, investigators or any other participant in the study. A frequentist monitoring approach will be used for evaluating the event rate ratio to ensure that the assumptions are accurate and the sample size continues to be appropriate for assessing superiority. The DSMB may recommend changes to the enrollment target if pretrial assumptions prove inaccurate.

Rationale for Study Design

A randomised, parallel group, double blind design is standard in phase III efficacy studies. Interim statistical analyses conducted by an independent Data Safety Monitoring Board will ensure that the trial can be closed if shown to be futile or resized if it turns out that the assumptions made about the primary endpoint in the control group are inaccurate.

Study Sites, Duration and Recruitment Rates

This is an international trial with recruitment across approximately 100 sites. The recruitment rates are estimated to be approximately 0.5 to 4 patients per site per month. Since this is a survival study patients are expected to be on study for a median time of 12 months.

Justification of the Proposed Dosing Regimen

In the TroVax® phase I study four dose levels were studied (1×10⁸ TCID50/ml, 2×10⁸TCID50/ml, 5×10⁸TCID50/ml, and 1×10⁹TCID50/ml) and two different routes of administration, intramuscular and intradermal, were compared. There was no clinically or statistically significant difference in peak immune response though the highest dose produced a slightly earlier antibody response. No difference was observed between the routes of administration in terms of antibody response. All doses and routes were well tolerated with only local injection site reactions which were of similar frequency. In view of a trend to an earlier antibody response the dose of 1×10⁹TCID50/ml was selected.

In subsequent phase II studies involving >70 patients, a dose level of 1×10⁹TCID50/ml was used and safety, tolerability and immunogenicity were confirmed.

In this study, TroVax®/placebo is administered at weeks 1, 3, 6, 9, 13, 17, 21, 25, 33, 41, 49, 57 and 65. This frequency is influenced by experience gained in phase II studies in patients with renal or colorectal cancer where TroVax® was co-administered with either combination chemotherapy, IL-2 or IFNα.

Study Population

Patient Recruitment

A total of 700 patients with clear cell renal carcinoma will be enrolled in the study. Eligible patients will have had the primary tumour surgically removed.

Patients will receive one of the following defined standards of care:
subcutaneous low dose IL-2
interferon alpha (excluding pegylated IFNα)
sunitinib

The choice of first-line standard of care for each patient will be made by the patient's physician based on normal clinical criteria, local standard of care, and local regulatory and reimbursement status or economic availability. Once treatment is selected, patients will be randomised to TroVax® or placebo.

Patients will be recruited internationally. Patients of all ethnic groups are eligible for the study.

Entry Criteria

Patients who meet the following inclusion criteria and none of the exclusion criteria will be included in this study.

Inclusion Criteria

Signed informed consent. The patient must be competent to give written informed consent and comply with the protocol requirements.

Locally advanced or metastatic, histologically proven clear cell renal carcinoma.

Primary tumour surgically removed (some residual advanced primary tumour may remain).

At least four weeks post surgery or radiotherapy (defined from time of randomisation.)

First-line. No prior therapy for renal cancer except surgery or radiotherapy.

Measurable disease.

Aged 18 years or more.

Patient expected to survive a minimum of 12 weeks (i.e. in the opinion of the investigator there is a >90% probability that the patient will survive >12 weeks if treated with the selected standard of care).

Free of clinically apparent autoimmune disease (including no prior confirmed diagnosis or treatment for autoimmune disease including Systemic Lupus Erythematosis, Grave's disease, Hashimoto's thyroiditis, multiple sclerosis, insulin dependant diabetes mellitus or systemic (non-joint) manifestations of rheumatoid disease).

Total white cell count ≧3×109/L and lymphocyte count ≧1×109/L.

Serum creatinine ≦1.5 times the upper limit of normal.

Bilirubin ≦2 times the upper limit of normal and an SGPT of ≦4 times the upper limit of normal.

Women must be either post menopausal, or rendered surgically sterile or, if of child bearing potential, must have been practising a reliable form of contraception (oral contraception+a barrier method) for at least three months prior to the first dose of TroVax® and must continue while they are being treated with TroVax®. Men must practise a reliable form of contraception (barrier or vasectomy) while they are being treated with TroVax®.

No acute changes on 12-lead ECG.

Ejection fraction documented as not less than 45% or no clinical suspicion that cardiac ejection fraction is less than 45%. (If clinical suspicion exists the ejection fraction should be measured according to local site procedures).

Karnofsky performance status of ≧80%.

Exclusion criteria

Cerebral metastases. (Known from previous investigations or clinically detectable).

Previous exposure to TroVax®.

Serious infections within the 28 days prior to entry to the trial.

Known to test positive for HIV or hepatitis B or C.

Life threatening illness unrelated to cancer.

History of allergic response to previous vaccinia vaccinations.

Known allergy to egg proteins.

Known hypersensitivity to neomycin.

Participation in any other clinical trial of a licensed or unlicensed drug within the previous 30 days or during the course of this trial.

Previous malignancies within the last 10 years other than successfully treated squamous carcinoma of the skin or in situ carcinoma of the cervix treated with cone biopsy.

Previous history of major psychiatric disorder requiring hospitalization or any current psychiatric disorder that would impede the patient's ability to provide informed consent or to comply with the protocol.

Oral corticosteroid use unless prescribed as replacement therapy in the case of adrenal insufficiency.

Ongoing use of agents listed in locally approved prescribing information as causing immunosuppression.

Prior history of organ transplantation.

Pregnancy or lactation.

Withdrawal Criteria

In accordance with applicable regulations, a patient has the right to withdraw from the study at any time and for any reason without prejudice to his or her future medical care by the physician or at the institution.

If a patient is withdrawn from treatment with TroVax®/placebo because of an adverse event (AE), the event will be followed up until it has resolved or has stabilized. Because this is a survival study patients should continue to be followed until death to document subsequent treatment and survival status

In addition to AEs, other reasons for removal of patients from the study would be the patient's withdrawal of consent. Should this happen, since this is a survival study, the patient's physician must request consent from the patient for survival follow up.

Withdrawal from the study, and reason for withdrawal, must be documented in the CRF.

Because the primary endpoint of this study is survival and all randomised patients will be included in the primary or secondary endpoint analysis patients who wish to withdraw from all other study related procedures for any reason should be asked whether they would consent to follow up limited to documenting their subsequent management and survival status. If they agree, a new informed consent form should be used to document consent to such follow up treatment plan and methods. The Study Schedule is set out below as Table 5:

| | Baseline | Wk 1 | Wk 2 | Wk 3 | Wk 4 | Wk 5 | Wk 6 | Wk 7 | Wk 8 | Wk 9 | Wk 10 | Wk 11 | Wk 12 | Wk 13 | Wk 14 | Wk 15 | Wk 16 | Wk 17 | Wk 18 |
|---|---|---|---|---|---|---|---|---|---|---|---|---|---|---|---|---|---|---|---|
| TroVax ®/ | | X | | X | | | X | | | X | | | | X | | | | X | |
| Placebo day | | | | | | | | | | | | | | | | | | | |
| 1/wk | | | | | | | | | | | | | | | | | | | |

| | | Patient receive only one of the following treatments | | | | | | | |
|---|---|---|---|---|---|---|---|---|---|
| Patients only | IL-2 | | X | X | X | X | X | X | Continue with 6 weeks subcutaneous IL-2 followed by 2 weeks without |
| receive one of | Treatment | | | | | | | | IL-2 every 8 weeks until tumour progression or week 46 (whichever is first) |
| these treatments | days 1-5 | | | | | | | | |
| | each wk | | | | | | | | |

| OR | | | | | |
|---|---|---|---|---|---|
| IFNα | Subcutaneous IFNα day 1, 3, and 5 of each week until tumour progression (refer to nationally | | | | |
| | approved prescribing information or institutional guidelines of use of IFNα for renal cancer). | | | | |
| OR | | | | | |
| sunitinib | X | X | X | X | Continue with 4 weeks on sunitinib then 2 weeks without sunitinib every 6 weeks |
| | | | | | until tumour progression. (see nationally approved sunitinib prescribing information) |

Patients receive all the following procedures

| Consent form | X | | | | | | | | | | | | | | | | | | |
|---|---|---|---|---|---|---|---|---|---|---|---|---|---|---|---|---|---|---|---|
| Randomisation | | X | | | | | | | | | | | | | | | | | |
| Medical History | X | | | | | | | | | | | | | | | | | | |
| Physical examination | X | | | | | | X | | | | | | | X | | | | | |
| Blood for Immuno | X | | | | | | | X | | | X | | | | | | | | |
| (10 ml) | | | | | | | | | | | | | | | | | | | |
| Weight, BP, Pulse, | X | X | | X | | | X | | | X | | | | X | | | | X | |
| Temp | | | | | | | | | | | | | | | | | | | |
| CBC/Diff/Plts | X | X | | X | | | X | | | X | | | | X | | | | X | |
| Chemistry Panel | X* | X | | X | | | X | | | X | | | | X | | | | X | |
| CT or MRI Chest, Abd, | X | | | | | | | | | | | | | | | | | | |
| Pelvis | | | | | | | | | | | | | | | | | | | |
| 12 lead ECG | X | | | | | | | | | | | | | | | | | | |
| Echocardiogram+ | X | | | | | | | | | | | | | | | | | | |
| Karnofsky | X | | | X | | | X | | | X | | | | X | | | | X | |
| Tumour histopathology | X | | | | | | | | | | | | | | | | | | |
| Pregnancy Test | X | Prior to TroVax ®/placebo if any possibility of pregnancy | | | | | | | | | | | | | | | | | |
| (if applicable) | | | | | | | | | | | | | | | | | | | |
| QOL | X | | | | | | X | | | X | | | | X | | | | X | |
| Concomitant Therapy | Record each visit that patient receives TroVax ®/placebo | | | | | | | | | | | | | | | | | | |
| AEs | Throughout the study while patient receiving TroVax ®/placebo and 30 days after | | | | | | | | | | | | | | | | | | |
| Subsequent renal | Record other renal cancer treatment once patient is not receiving TroVax ®/placebo | | | | | | | | | | | | | | | | | | |
| cancer Rx | | | | | | | | | | | | | | | | | | | |
| Surival status/date | Record on each visit that patient receives TroVax ®/placebo and every 12 weeks thereafter. If patient | | | | | | | | | | | | | | | | | | |
| of death | does not return to clinic seek survival status and date of death as permitted by patient consent | | | | | | | | | | | | | | | | | | |
| | Wk 19 | Wk 20 | Wk 21 | Wk 22 | Wk 23 | Wk 24 | Wk 25 | Wk 26 | Wk 27 | Wk 28 | Wk 29 | Wk 30 | Wk 31 | Wk 32 | Wk 33 | Wk 34 | Wk 35 | Wk 36 | Wk 37 |
| TroVax ®/ | | | X | | | | X | | | | | | | | X | | | | |
| Placebo day 1 | | | | | | | | | | | | | | | | | | | |

| | | Patient receive only one of the following treatments |
|---|---|---|
| Patient is only | IL-2 | Continue with 6 weeks subcutaneous IL-2 followed by 2 weeks without IL-2 |
| | Treatment | every 8 weeks until tumour progression or week 46 (whichever is first) |
| | days 1-5 | |
| | each wk | |
| | OR | |
| | IFNα | Subcutaneous IFNα day 1, 3, and 5 of each week until tumour progression (refer to nationally approved |
| | | prescribing information or institutional guidelines of use of IFNα for renal cancer). |
| | OR | |
| | sunitinib | Continue with 4 weeks on sunitinib then 2 weeks without sunitinib every 6 weeks until |
| | | tumour progression. (see nationally approved sunitinib prescribing information) |
| | | Patients receive all the following procedures |

| Physical examination | | | | | | | | X | | | | | | | X | | | | |
|---|---|---|---|---|---|---|---|---|---|---|---|---|---|---|---|---|---|---|---|
| Weight, Bp, Pulse, Temp | | | X | | | | X | | | | X | | | | X | | | | |
| CBC/Diff/Plts | | | X | | | | X | | | | X | | | | X | | | | |
| Chemistry Panel | | | X | | | | X | | | | X | | | | X | | | | |
| CT or MRI Chest Abd, | | | | | | | | X | | | | | | | X | | | | |
| Pelvis | | | | | | | | | | | | | | | | | | | |
| Karnofsky | | | X | | | | X | | | | X | | | | X | | | | |
| QOL | | | X | | | | X | | | | X | | | | X | | | | |
| Pregnancy Test | Prior to TroVax ®/placebo if any possiblility of pregnancy | | | | | | | | | | | | | | | | | | |
| (if applicable) | | | | | | | | | | | | | | | | | | | |
| Concomitant Therapy | Record on each visit that patient receives TroVax ®/placebo | | | | | | | | | | | | | | | | | | |
| AEs | Throughout the study while patient receiving TroVax ®/placebo and 30 days after | | | | | | | | | | | | | | | | | | |
| Subsequent renal | Record other renal cancer treatment once patient is not receiving TroVax ®/placebo | | | | | | | | | | | | | | | | | | |
| cancer Rx | | | | | | | | | | | | | | | | | | | |
| Survival status/date of | Record on each visit that patient receives TroVax ®/placebo and every 12 weeks thereafter. If patient | | | | | | | | | | | | | | | | | | |
| death | does not return to clinic seek survival and date of death as permitted by patient consent | | | | | | | | | | | | | | | | | | |
| | Wk 38 | Wk 39 | Wk 40 | Wk 41 | Wk 42 | Wk 43 | Wk 44 | Wk 45 | Wk 46 | Wk 47 | Wk 48 | Wk 49 | Wk 50 | Wk 51 | Wk 52 | Wk 53 | Wk 54 | Wk 55 | Wk 56 |
| TroVax ®/Placebo day 1 | | | | X | | | | | | | | X | | | | | | | |

| | | Patients receive only one of the following treatments | |
|---|---|---|---|
| Patients only | IL-2 | Continue with 6 weeks subcutaneous IL-2 | No furthur IL-2 |
| receive one of | Treatment | followed by 2 weeks without IL-2 every 8 weeks | |
| these treatments | days 1-5 | until tumour progression or week 46 | |
| | each wk | (whichever is first). | |

| | |
|---|---|
| OR | |
| IFNα | Subcutaneous IFNα day 1, 3, and 5 of each week until tumour progression (refer to nationally approved |
| | prescribing information or institutional guidelines of use of IFNα for renal cancer). |
| OR | |
| sunitinib | Continue with 4 weeks on sunitinib them 2 weeks off sunitinib every weeks until |
| | tumour progression. (see nationally approved sunitinib prescribing information) |

| | Patients receive all the following procedures | | | | | | | | | | | |
|---|---|---|---|---|---|---|---|---|---|---|---|---|
| Physical examination | | | | X | | | | | | | | X |
| Weight, BP, Pulse, Temp | | | | X | | | | | | | | X |
| CBC/Diff/Plts | | | | X | | | | | | | | X |
| Chemistry Panel | | | | X | | | | | | | | X |
| Karnofsky | | | | X | | | | | | | | X |
| QOL | | | | X | | | | | | | | X |
| Pregnancy Test | Prior to TroVax ®/placebo if any possiblility of pregnancy | | | | | | | | | | | |
| (if applicable) | | | | | | | | | | | | |
| Concomitant Therapy | Record on each visit that patient receives TroVax ®/placebo | | | | | | | | | | | |
| AEs | Throughout the study while patient receiving TroVax ®/placebo and 30 days after | | | | | | | | | | | |
| Subsequent renal | Record other renal cancer treatment once patient is not receiving TroVax ®/placebo | | | | | | | | | | | |
| cancer Rx | | | | | | | | | | | | |
| Survival status/date of | Record on each visit that patient receives TroVax ®/placebo and every 12 weeks thereafter. If patient | | | | | | | | | | | |
| death | does not return to clinic seek survival and date of death as permitted by patient consent | | | | | | | | | | | |
| | Wk 57 | Wk 58 | Wk 59 | Wk 60 | Wk 61 | Wk 62 | Wk 63 | Wk 64 | Wk 65 | Wk 66 | Subsequent weeks | |
| TroVax ®/Placebo day 1 | X | | | | | | | | X | | | |

| | | |
|---|---|---|
| | | Patients receive only one of the following treatments |
| Patients only | IL-2 | No further IL2 |
| receive one of | Treatment | |
| these treatments | days 1-5 | |
| | each wk | |
| | OR | |
| | IFNα | Subcutaneous IFNα day 1, 3, and 5 of each week until tumour progression (refer to nationally approved |
| | | prescribing information or institutional guidelines for use of IFNα for renal cancer). |
| | OR | |
| | sunitinib | Continue with 4 weeks on sunitinib then 2 weeks off sunitinib every six weeks until |
| | | tumour progression. (see nationally approved sunitinib prescribing information) |
| | | Patients receive all the following procedures |

| | | | |
|---|---|---|---|
| Physical examination | X | X | Continue follow-up for survival |
| Weight, BP, Pulse, Temp | X | X | Record subsequent thereapy for renal cancer |
| CBC/Diff/Plts | X | X | |
| Chemistry Panel | X | X | |
| Karnofsky | X | X | |
| QOL | X | X | |

| | |
|---|---|
| Pregnancy Test | Prior to TroVax ®/placebo if any possiblility of pregnancy |
| (if applicable) | |
| Concomitant Therapy | Record on each visit that patient receives TroVax ®/placebo |
| AEs | Throughout the study while patient receiving TroVax ®/placebo and 30 days after |
| Subsequent renal | Record other renal cancer treatment once patient is not receiving TroVax ®/placebo |
| cancer Rx | |
| Survival status/date of | Record on each visit that patient receives TroVax ®/placebo and every 12 weeks thereafter. |
| death | If patient does not return to clinic seek survival and date of death as permitted by patient consent |

If clinically indicated * including LDH baseline
Timing of all TroVax ® injections +/−3 days. Timing of all laboratory and clinical observations must remain the same relative to TroVax ®. Week 26 scan may vary by +/− 7 days.

Allocation of Treatments and Randomisation Procedures

Treatment (TroVax® or placebo) will be allocated based on stratified randomisation. The primary objective of stratification will be to ensure that the distribution of first-line standard of care treatment is balanced between the two study arms. Secondary objectives of stratification will be to establish balance between the treatment arms with regard to a prognostic index (Motzer score) and geography.

Motzer et al demonstrated in a series of 670 patients with advanced renal cell carcinoma that survival correlated with five prognostic factors: Karnofsky performance status (<80%), high lactate dehydrogenase (LDH) level (>1.5 times the upper limit of normal), low haemoglobin level (less than the lower limit of the gender normal), high corrected serum calcium level (>10 mg/dL), and absence of nephrectomy. The higher the number of positive factors the worse the prognosis. Inclusion criteria for this study require a baseline Karnofsky performance status ≧80% and prior excision of the primary tumour. During the randomisation procedure the patient's haemoglobin level (plus gender), LDH and serum calcium will be requested to ensure that the treatment arms are balanced with regard to these prognostic variables.

A telephone based interactive voice responsive system will be used. Patients will be registered into the study using an Interactive Voice Responsive System (IVRS). Treatment allocation (TroVax® or placebo) and patient registration will only occur after the Investigator has registered the standard of care therapy allocated to the patients and confirmed that the patient meets all inclusion/exclusion criteria. All randomised patients will be included in Intent to Treat (ITT) analyses.

Instruction on access and use of the IVRS service including local telephone access number, script of the randomisation questions in local language and help desk numbers will be issued separate from the protocol.

Study Medication Administration

Patients included in this trial should receive TroVax® or placebo plus one of the following first-line standards of care treatment options: IL-2 (low dose), interferon a or sunitinib. No other form of immunotherapy, chemotherapy, or radiotherapy should be administered between entering the study and tumour progression. Other concurrent medication may be used as detailed in “Other Concurrent Treatments” below. Following tumour progression patients may receive whatever chemotherapy, radiotherapy, cytokine therapy or other therapy is indicated for further management or palliation of the tumour. All such therapy should be recorded on the patient's case report form as the patient continues to be followed for survival.

Administration of TroVax®/Placebo

Prior to administering the vaccine, obtain the prospective patient's vaccination history and determine whether the individual had any previous reactions to any vaccine including TroVax®.

All immunisations of TroVax®/placebo will be given by intramuscular injection into the deltoid muscle of the upper arm.

All patients will receive the treatment in a side-room away from contact with other patients. The formulation will be delivered to this side-room. TroVax®/ Placebo are presented as lyophilised material. Detailed instructions will be provided to the pharmacist for reconstitution. TroVax® must be re-suspended by adding 1.2 mL of water for injection. The resulting solution will appear opalescent. One mL volume of the solution is then withdrawn into a syringe and injected into the patient. The injection will either be drawn up at the bedside by the person administering the dose, or in the pharmacy and delivered to the bedside in a syringe depending upon local circumstances. Prior to injection the check number of the dose must be confirmed, using IVRS, by either the pharmacist or another responsible individual.

Under no circumstances must the reconstituted material be allowed to stand for more than two hours at room temperature. If this does occur, the material must be rejected and IVRS notified.

The skin will be swabbed with ethanol and the injection will be given intramuscularly. Following this, the injection site will be covered with an occlusive bandage. This bandage will be removed before the patient is discharged from hospital.

Please note: The maximum immunological response to TroVax® dose not usually occur until the patient has received at least three injections. Disease stabilisation or late tumour responses have been reported with various cancer vaccines. It is not established whether continuing TroVax® despite early progression will confer therapeutic benefit. If tumour progression is observed but the patient is tolerating TroVax®/placebo and their performance status remains at a Karnofsky score >60% they should be requested to continue receiving TroVax®/placebo until they have received a minimum of eight injections. Continuation beyond this point is permitted at the discretion of the investigator and patient.

Patients should remain under medical observation for one hour following injection with TroVax®/placebo.

Adequate treatment provisions, including epinephrine injection (1:1000), should be available for immediate use should an anaphylactic reaction occur.

All healthcare staff handling TroVax® or materials contaminated by it must wear an apron, gloves, mask, and protective goggles. All materials potentially contaminated with TroVax® e.g. syringes, swabs, bandages, must be destroyed by incineration, or local equivalent, in accordance with hospital policy on genetically modified materials. Certificates of Destruction, or equivalent, must be completed for the used and unused vials, and copies maintained in the Trial File.

Administration of IL-2

IL-2 (Chiron or locally approved manufacturer) will be given by subcutaneous injection. The lyophilised material (22 million units) must be reconstituted in 1.2 mL of diluent after which it will have a shelf life of 48 hours when kept refrigerated at 2-8° C. The dosage schedule will be an initial dose of 250,000 U/Kg/dose (with an upper limit of 22 million units/dose) for 5 days out of 7 in week 1 of each cycle followed by 125,000 U/kg/dose (with an upper limit of 11 million units/dose) for 5 days in each of weeks 2-6 of each cycle. There will then be a two week recovery period before the next cycle of IL-2 commences. Once reconstituted a vial may be used for two injections when these are given on consecutive days. The dose used should be recorded in the Case Report Form.

Administration of IFNα

IFNα will be administered once a day as a subcutaneous injection three times per week on days 1, 3 and 5 of each week. (Note: Pegylated IFNα is not included as a standard of care option in this protocol. No safety or immunological activity data are currently available on the concomitant use of TroVax® and pegylated IFNα).

Unless tumour progression is noted the patient should be treated for a minimum of 12 weeks. Treatment may be continued until tumour progression at the discretion of the investigator.

Doses of IFNα used by different treatment centres depend on local Regulatory Authority approved label text, and manufacturer. The dose used in this study should reflect local standard of care but should be targeted between 9 million International Units (IU) and 18 million IU three times per week. Lower doses should be used during the first (and depending on final target dose) the second week. The actual schedule used will be recorded on the Case Report Form.

For further information on IFNα please refer to the nationally approved Package Insert or Summary of Product Characteristics produced by the local license holder.

For evaluation of patients for clinical benefit from the treatment please see study schedule. Patients who are benefiting from treatment are eligible for further treatment. Thereafter, therapy will continue until criteria for progressive disease are met or up to an additional 12 months.

Administration of Sunitinib

Sunitinib capsules are supplied as printed hard shell capsules containing sunitinib malate equivalent to 12.5 mg, 25 mg or 50 mg of sunitinib and should be handled according to the manufacturers instructions. The recommended dose of sunitinib for advanced Renal Cell Cancer is one 50 mg oral dose taken once daily, on a schedule of 4 weeks on treatment followed by 2 weeks off. Sunitinib may be taken with or without food. The schedule used should be recorded in the Case Report Form.

Treatment should continue until tumour progression or until unacceptable toxicity occurs.

Administration of Other Concurrent Treatments

All other concurrent medications will be recorded in detail in the CRF during the treatment. This information may be used to assist interpretation of any report adverse events. If a patient has discontinued TroVax®/placebo and other renal cancer treatments are used, then a simple checklist in the CRF will be used to record the type of treatment; this information may be used to assist interpretation of survival data and management of the patient following the selected standard of care therapy.

Medication intended to relieve symptoms will be prescribed at the discretion of the Investigator and recorded in the Case Report Form (CRF). Medications prescribed by the patient's family practitioner will also be noted in the CRF. The patients should also keep a record of any over the counter medicines consumed and these should be noted in the CRF.

Therapies considered necessary for the subject's well being may be administered at the discretion of the investigator. These will be recorded in the Case Report Form.

Supportive care to mitigate known adverse events or complications of concomitant standard of care may be administered at the physician's discretion including antipyretics, non-steroidal anti-inflammatories, anti-emetics etc. Oral, intramuscular or intravenous steroids should not be used except where required to manage life threatening emergencies. Supportive care will be reported in the Case Report Form.

Management of Disease Progression

If disease progression is noted during the study, and other anticancer medications are required, the IL-2, IFNα, or sunitinib should be stopped. The selection of subsequent antitumour therapy is not specified by this protocol and is at the discretion of the patient and his or her physician.

In the event of tumour progression the patients should remain within the study (unless they request to withdraw). This is for two reasons:

This is a survival study and patients need to be followed for survival data.

The maximum immunological response to TroVax® does not usually occur until the patient has received at least three injections. Disease stabilisation or late tumour responses have been reported with various cancer vaccines. It is not established whether continuing TroVax® despite early progression will confer therapeutic benefit. Therefore if tumour progression is observed but the patient is tolerating TroVax®/placebo and their performance status remains at a Karnofsky score >60% they should be requested to continue receiving TroVax®/placebo until they have received a minimum of eight injections of the study preparation. Continuation on study beyond this point to receive all TroVax®/placebo injections is permitted at the discretion of the investigator or patient.

Specific Procedures

Screening and Selection Procedures

A screening log must be maintained for all patients screened for entry to the study including, if applicable, the reason for not entering the study.

Inclusion/exclusion criteria are listed in the section titled Entry Criteria (above) and the study schedule.

Imaging/Diagnostic

Within 2 weeks of screening, and prior to receiving study drug metastases will be documented using chest, abdominal and pelvic CT scans according to defined guidelines contained in a Site Operations Manual. This will enable a possible independent review at a later time. An MRI or CT scan of the brain will also be obtained if there is a clinical suspicion of cerebral metastases.

Clinical and Laboratory/Diagnostic

For screening, these are required within 14 days before the first TroVax®/Placebo injection:
History and physical examination, including height, weight, and vital signs.
Karnofsky performance status.
Quality of life (QLQ30,EuroQOL) will be evaluated.
12 lead EGC (for all patients) and Echocardiogram only if clinically indicated
Clinical pathology tests (Full blood count with differential white cell and platelet counts, urea and electrolytes, liver function tests (total bilirubin, AST, ALT, alkaline phosphatase), serum proteins, calcium, phosphate, uric acid and creatinine). In addition, at baseline LDH must be measured.
Pregnancy test (for women of reproductive potential—including those whose last menstrual period was within the last two years). At screening this will be a serum test but at all other time points this will be a urine test.
If available, tumour tissue from earlier biopsies will be obtained. (To be batched and tested at a later date for the presence of tumour antigens.)

All clinical laboratory tests will be conducted by a suitably qualified central laboratory.

Samples for Immunology

10 mL blood samples will be required. These samples are to be placed in a heparinised blood collection tube and are to be processed immediately by a suitably qualified central laboratory. The samples will then be analysed by Oxford BioMedica, or designee, according to their SOPs.

Study Materials

### TroVax® /Placebo

TroVax®/Placebo will be supplied by Oxford BioMedica Ltd.

Packaging and labelling and additional information. Packaging and labelling will be in accordance with Good Manufacturing Practice (GMP) for clinical trials. Each vial will bear a label conforming to national regulations for an Investigational Medicinal Product. The ouer carton labelling will also bear a label conforming to national regulations for an Investigational Medicinal Product.

Investigators and pharmacists should note that the clinical trial supplies may only be used for the clinical trial for which they are indicated. They must not be employed for any other trial, whether of TroVax® or not, or for any other clinical use.

Additional information may be found in the current version of the Investigators Brochure.

Storage and Disposition of Study Medications

TroVax®/placebo must be stored in a locked fridge between 2° C. to 8° C. (36° F. to 46° F).in the hospital pharmacy, or other comparable secure location. It must be stored in such a way that it cannot be mixed up or confused with other medications, be they clinical trial supplies or medicines for routine clinical use.

Dispensing will be documented by completing a log with the date of dispensing and the patient details. Used vials should be stored in labelled biohazard bags or containers prior to reconciliation by the trial monitor.

At each visit, the clinical trial monitor will review the drug-dispensing log and reconcile it with the unused vials (if available due to local procedures). All unused vials will be destroyed on site in accordance with procedures for destruction of genetically modified waste and destruction will be documented appropriately. A copy of the Certificate of Destruction will be lodged in the site Trial File.

Precautions/Overdose

TroVax® is contraindicated in patients who have previously had hypersensitive reactions to TroVax®, vaccinia vaccinations, egg proteins or neomycin. Patients should remain under medical observation for one hour following injection with TroVax. Adequate treatment provisions, including epinephrine injection (1:1000), should be available for immediate use should an anaphylactic reaction occur. TroVax® is also contraindicated in patients who are pregnant or lactating.

Although highly unlikely, it is possible that an autoimmune response against the pituitary or gut might occur since these organs showed sporadic low level staining for 5T4 in in vitro experiments. Studies in over 100 patients receiving approximately 450 doses of TroVax® have not indicated any laboratory or clinical signs or symptoms suggestive of compromised pituitary function. However, the Investigators should be aware of the preclinical finding.

All healthcare staff handling TroVax® or materials contaminated by it must wear an apron, gloves, a mask and protective goggles. Pregnant healthcare staff must not handle either TroVax® or materials contaminated with TroVax®.

No cases of TroVax® overdose have been reported. No active medical intervention is know to be required in the event of overdose. The patient should be observed for as long as is considered appropriate by the investigator/physician based on the patient's clinical condition and supportive care given if required.

### IL-2

II-2 is available commercially from Chiron or a local manufacturer. The lyophilised material (22 million units) must be reconstituted in 1.2 mL of diluent after which it will have a shelf life of 48 hours when kept refrigerated.

Current prescribing information should be reviewed prior to administering IL-2.

### IFNα

IFNα is available commercially from a number of manufactures. Only commercially available material approved by the competent national regulatory authority should be used in this study

IFNα may be supplied in single use prefilled syringes or in multiuse prefilled “pens”. Patients will be instructed to self administer the IFNα in accord with approved package insert and patient information leaflet by appropriately qualified medical, nursing or pharmacy staff. Reconstitution is not required.

IFNα should be stored at 2° to 8° C. (36° F. to 46° F.).

Current prescribing information should be reviewed prior to administering IFNα.

Sunitinib

Sunitinib is supplied as 12.5 mg, 25 mg and 50 mg capsules which should be administered according to the manufacturer's instructions (Pfizer).

Other Study Supplies

Case report forms (CRFs) will be used in this study (see Data Collection section below). Quality of life questionnaires EuroQOL and QLQ30 and laboratory kits will also be supplied. The Principal Clinical Investigator and Co-Investigators must keep all CRF supplies, both completed and blank, in a secure place.

Adverse Events

Adverse Event Definition

An adverse event is any untoward medical occurrence in a patient or clinical investigation subject administered a pharmaceutical product and which does not necessarily have to have a causal relationship with the treatment. All adverse events must be described in the appropriate section of the CRF and their severity and putative relationship to the study medication noted. Definitions of severity are as follows:
Mild: does not interfere with the conduct of the study, resolves spontaneously, does not need medication or any other therapy.
Moderate: requires treatment, interferes temporarily with the conduct of the study.
Severe: forces withdrawal from the study
Serious: death, life threatening, requires or prolongs hospitalisation, results in persistent or significant disability/incapacity, overdose, or is a congenital anomaly/birth defect

Definitions of relationship to study medication are as follows:
Unrelated: bears no relation to timing of medication, similar to symptoms or signs expected in the disease process, does not recur on re-challenge.
Possibly: bears relation to timing of medication, similar to symptoms or signs expected in the disease process, does not recur on re-challenge.
Probably: bears clear relation to timing of medication, distinct from symptoms or signs expected in the disease process, does not recur on re-challenge.
Definitely: bears clear relation to timing of medication, distinct from symptoms or signs expected in the disease process, recurs on re-challenge.

Adverse events may also be expected or unexpected. Adverse events are to be considered expected if listed in the Investigator Brochure.

Serious Adverse Event (SAE) and Serious Adverse Reaction (SAR) Definition

Investigators are required to notify Oxford BioMedica's pharmacovigilance service provider (PAREXEL) immediately if a patient has a reportable serious adverse event. A serious adverse event (SAE) is defined by ICH-GCP as:

Death (death due to progressive renal cancer is the primary endpoint of this study and should not be reported as an adverse event unless in the opinion of the investigator the study medication (TroVax®/placebo) may possibly, probably or definitely have contributed to or hastened death)

Life Threatening

Requires or prolongs hospitalisation

Results in persistent or significant disability/incapacity

Congenital anomaly/birth defect

Other medically important condition starting or worsening during the study.

The investigator must also complete as much as possible of the serious adverse event form in the Case Report Form (CRF) and transfer it to Oxford BioMedica's pharmacvigilance service provider (PAREXEL) not later than 24 hours after the even becomes known to the investigator or his/her staff.

As further information or follow up information becomes available the investigator should document this and amend any previous report if appropriate. This information should be transferred to Oxford BioMedica's pharmacovigilance service provider (PAREXEL) using the serious adverse event form in the CRF.

PAREXEL will report all serious, related, and unexpected adverse events to all relevant Regulatory Authorities in accordance with local regulations.

Further instructions on the documentation and transfer of information to permit full compliance with national and international pharmacovigilance requirements and Good Clinical Practice together with training for investigator staff will be provided separate to this protocol.

General Requirements

This study will utilize the Common Terminology Criteria for Adverse Events Version 3 to determine the severity of the reaction for adverse event reporting.

Reporting requirements and procedures depend upon:
whether agents are suspected of causing the adverse event,
whether the possibility of such an adverse event was reported in the protocol, consent form, or manufacturer's literature (expected or unexpected adverse event),
the severity or grade of the adverse event.

Withdrawals due to Adverse Events

If a patient is withdrawn from treatment because of an adverse event (AE), the patient will be followed up until the AE is resolved or has stabilised. Because the primary endpoint of this study is survival the patient will continue to be followed for survival status even if trial therapy was withdrawn.

Withdrawal from the study, and reason for withdrawal, must be documented in the CRF.

Since the primary endpoint of this study is survival and all randomised patients will be included in the analysis of the primary endpoint. Patients who wish to withdraw from all other study related procedures should be asked whether they would consent to allow follow up limited to establishing their survival status. If they agree, a new consent form to document this consent but withdrawal from all other study procedures should be completed.

Pregnancy

Patients should be advised that they or their partner should avoid becoming pregnant during the study.

Patients of reproductive potential should be taking contraceptive measures as required by the relevant inclusion criterion (as stated above).

If a patient does become pregnant she should immediately inform the investigator who should document this on the adverse events page of the CRF. The Investigator should provide necessary counselling for the patient. The Investigator should follow the pregnancy to its conclusion. Spontaneous abortion or foetal abnormality or abnormal birth should be reported as serious adverse events as described above.

Management of Toxicity

The NCI Common Terminology Criteria for Adverse Events v3.0 (CTCAE) will be utilized (see Appendix A). Toxicity will be evaluated on every patient visit.

All toxic events should be managed with optimal supportive care, including transfer to the Intensive Care Unit if appropriate.

TroVax®/Placebo Management of Toxicity

No dose reductions of TroVax®/placebo are permitted. Paracetamol/acetaminophen may be used to manage transient pyrexia or local discomfort following injection If the patient is unable to tolerate TroVax®/placebo at the protocol dose TroVax®/placebo should be discontinued but the patient should continue to be followed for survival data.

Standard of Care Management of Toxicity

Toxicity associated with standard of care therapy should be managed according the nationally approved Package Insert or Summary of Product Characteristics and accepted medical practice. Dosage may be reduced or withdrawn at the discretion of the Investigator.

Data Management and Statistical Analysis

Overview of the Study Design

The DSMB will be responsible for preparing the formal monitoring rules for this study; a general overview of the monitoring program is described in this section of the protocol. Oxford BioMedica will provide guidance to the DSMB, however the Board is an independent body and will be charged with preparing the formal monitoring and stopping rules for the study. This parallel-designed study contains a series of planned interim assessments for futility, and to ensure the planning elements relative to attrition and the primary endpoint remain consistent. The initial interim assessment will take place after 50 patients (25 patients per arm or ˜7% of the target population) have been randomised and followed for 8 weeks when the blood sampling for 5T4 antibodies following the third dose of TroVax® is scheduled to be performed. The intra-treatment group adverse event profiles, rates of attrition, and antibody response will be evaluated by the DSMB. Sample size estimates for this study are predicated on a one year survival.

Sample Size Estimates

Estimates were prepared to detect an absolute difference of ˜11% in survival at 1-year (base proportions: 50% to 61%); estimates are presented below in Table A.

TABLE A
Estimates Based on Overall Survival

| | | Total | | | Pro- | Pro- | |
|---|---|---|---|---|---|---|---|
| | Total | Re- | | | portion | portion | |
| | Sample | quired | | | Surv. | Surv. | Hazard |
| Power | Size (N) | Events | Alpha | Beta | (S1) | (S2) | Ratio |
| 0.80 | 691 | 309 | 0.05 | 0.2 | 0.500 | 0.605 | 0.725 |

A total sample size of ˜700 patients (split equally between the two groups), or 309 events, achieves 80% power to detect a hazard rate of 0.725 when the proportions surviving in each group are 0.500 and 0.605 at a significance level of 0.05 using a two-sided test. These estimates represent the initial framework for monitoring based on the log of the hazard ratio from the Cox Proportional Hazards regression model without adjusting for covariates.

Report Definitions

Power is the probability of rejecting a false null hypothesis.

Events are the number of deaths (from whatever cause) that must occur in each group.

Alpha is the probability of rejecting a true null hypothesis.

Beta is the probability of accepting a false null hypothesis.

S1 is the proportion surviving in group 1, S2 is the proportion surviving in group 2.

HR is the hazard ratio. It is calculated using Log(S2)/Log(S1).

This sample size would also be appropriate for detecting a minimum difference in median survival of ˜11.3 weeks, based on exponential survival times (Table B). Details used in preparing this estimate are presented below.

TABLE B
Comparing Median Survival (H0: Theta1 = Theta2. Ha: Theta1 <> Theta2)

| | | | Allocation | | | | | Theta1/ |
|---|---|---|---|---|---|---|---|---|
| Power | N1 | N2 | Ratio | Alpha | Beta | Theta1 | Theta2 | Theta2 |
| 0.80000 | 350 | 350 | 1.00000 | 0.05000 | 0.20000 | 48.0 | 59.3 | 0.80902 |

Report Definitions

Power is the probability of rejecting a false null hypothesis.

N1 is the number of failures needed in Group 1, N2 is the number of failures needed in Group 2.

Alpha is the probability of rejecting a true null hypothesis.

Beta is the probability of accepting a false null hypothesis.

Theta1 is the Mean Life in Group 1, Theta2 is the Mean Life in Group 2.

Patient Populations

The Intent to Treat (ITT) population will include all patients who are randomised.

The Modified Intent to Treat (MITT) population, will include all patients who receive three or more injections, or experience an adverse event directly attributable to the study medication resulting in discontinuation, prior to the third injection. Patients who fail to successfully receive three injections for reasons not directly associated with the study medication will not be included in this population.

The Per Protocol (PP) population includes only patients who met the inclusion and exclusion criteria and were treated in accord with the protocol requirements.

The primary efficacy analysis will be carried out using the ITT population. However, an exploratory analysis of the primary efficacy parameter will also be carried out using MITT population and the PP population.. All safety analyses will be carried out using the Intent to Treat population.

Monitoring of the Primary Endpoint

The DSMB may recommend stopping the trial early if presented with overwhelming evidence of efficacy.

Evidence would be deemed “overwhelming” if the one-sided P-value in favour of the active treatment derived from the Cox Proportional Hazards time-to-death model is less than 0.01%. The overall effect of treatment must also be considered clinically plausible by the DSMB.

P-values will be adjusted to maintain an overall one-sided P-value of 2.5% using the alpha-spending approach of Lan and Demets (Lan K K G and DeMets D L (1983) Discrete sequential boundaries for clinical trials. Biometrika 70: 659-663).

The DSMB will review at each meeting the number of patients lost to follow-up. If the number of patient lost to follow-up is high enough to compromise the objectives of the study the DSMB may either recommend terminating the study on the grounds that it will not effectively address its objective or alternatively resizing the study to permit the objective of the study to be appropriately address.

The DSMB may also recommend stopping the trial early if presented with evidence of futility. At each interim analysis the conditional power will be calculated. If, taking into account the whole clinical context, the DSMB considers the prospect of achieving a statistically significant result within a reasonable sample size to be unacceptably low, then the DSMB may recommend stopping the trial.

The methodology for study re-sizing will follow that of Li, Shih, Xie and Lu (Li G, Shih W J, Xie T and Lu J (2002) A sample size adjustment procedure for clinical trials based on conditional power. Biostatistics 3: 277-287).

Statistical Analyses

Unless otherwise stated, all statistical tests will be performed using 2-sided tests at the 5% significance level. Baseline is defined as the last observation before the initiation of the study related treatment. Continuous demographic parameters, such as the patient's age at the time of enrolment, will be summarised for the ITT population using descriptive statistics (N, mean, median, standard deviation, minimum and maximum value, and 95% 2-sided confidence limits) and compared between groups using a 2-sample t-test. Categorical parameters will be summarised as a proportion of the ITT population and compared using a 2-tailed Fisher's Exact test. Co-morbid risk factors will be summarised for the ITT population by treatment assignment and according to the type of variable (categorical, continuous) and compared between groups. Kaplan-Meier estimates for the time to death will be prepared based on the ITT population. Event rates at 12- and 24-months will be derived from the Kaplan-Meier estimates. The number and proportion of patients alive after each treatment cycle will be tabulated and summarised using 95% confidence intervals. Separate tables containing patient counts, percentages, and 95% binomial confidence intervals will be prepared based on risk factors. No data will be imputed for patients who withdrew prematurely from the study, or have missing values for specific parameters.

Univariate analyses will be prepared for each laboratory parameter and compared between groups using a 2-sample t-test. The proportion of patients found to have abnormal values considered clinically significant will be compared between treatment groups using a 2-tailed Fisher's Exact test. Laboratory shift tables containing patient counts and percentages will be prepared by treatment assignment, laboratory parameter, and time.

Demography

Patient demographic data will be summarised by type of variable; categorical data by counts and percentages and continuous variable by means, standard deviations, medians, minimum, maximum and numbers of patients.

Analysis of Efficacy Data

The standard covariates for the efficacy analyses are:

Geographical region (three groups: USA, European Union, Eastern Europe excluding European Union).

First line of standard care (three groups: IL-2, interferon-α, sunitinib)

Prognostic index (Motzer score). (Motzer score classifies patients into three prognostic groups: “favorable”, “intermediate” and “poor” based on an algorithm which considers pre-treatment performance status, LDH, haemoglobin, and corrected serum calcium. The inclusion and exclusion criteria preclude enrolment of the “poor” prognostic group. All eligible patients will be covered in the remaining two groups)

Primary

The primary endpoint is time to death. Time to death will be analyzed in the ITT population using a Cox Proportional Hazards regression model with terms for treatment and the standard efficacy covariates.

Secondary

The secondary efficacy endpoints will be analysed following the statistical procedures presented below.

Endpoint: The proportion of patients with progression free survival at 26 weeks (+/−1 week) based on radiological data in the ITT population.

The proportion of patients with progression free survival at 26 weeks (+/−1 week) relative to baseline will be analysed using a logistic regression model with terms for treatment and the standard efficacy covariates. Data will be analysed using the ITT population and adjudicated (blinded peer review).

Endpoint: Tumour response rates based on RECIST according to the investigator's reported interpretation of the radiological reports observed in the ITT population.

Both the rate and duration of tumour response will be compared between treatment groups. Response rates will be compared between treatment groups and analysed using a logistic regression model with terms for treatment and the standard efficacy covariates. The duration of response will be analysed using a Cox Proportional Hazards regression model with terms for treatment and the standard efficacy covariates.

Endpoint: The survival event rate ratio in the TroVax® arm versus the placebo arm in the MITT population, based on the log of the hazard ratio.

Time to death will be analysed using a Cox Proportional Hazards regression model with terms for treatment and the standard efficacy covariates. Survival curves for the proportion of patients remaining event-free will be estimated using the Kaplan-Meier method

Endpoint: Anti-5T4 serum antibody levels (additional measures of immune response including specific measures of cellular response will be investigated at some centres).

Qualitative antibody response to 5T4 within the active treatment group will be analysed as a main effect using a logistic regression model with terms for the standard efficacy covariates.

The analysis of the Quality of Life (QOL) parameters is discussed below.

Analysis of Adverse Event Data

Safety will be assessed using the Intent to Treat population. Adverse events will be coded using the MedDRA classification to give a preferred term and organ class for each event. Proportions of patients with adverse events will be presented. Tables of adverse events will be presented by organ class and also by organ class and preferred term. These tables will also include overall totals for adverse events within each body system and organ class. The number of patients with an event in each classification of severity and relationship to treatment within each treatment group will be tabulated. Serious adverse events and adverse events leading to withdrawal will be listed separately.

Treatment emergent and non-emergent events will be presented separately. Treatment emergent adverse events are defined as adverse events that had an onset day on or after the day of the first dose of study medication. Adverse events that have missing onset dates will be considered to be treatment emergent.

Adverse events will be listed by patient within groups showing time of onset, period of event, severity, relationship to disease and outcome.

QOL Parameters

Results from the QOL questionnaire (EuroQoL and QLQ30) will be presented for the ITT and Per-Protocol populations. Results from the QOL questionnaire will be analyzed using a generalised linear modelling approach based on maximum likelihood, treating patients as a random effect in the model. Terms will be included for the standard efficacy covariates.

Concomitant Medication

Concomitant medication will be listed by patient, treatment assignment, and study visit.

Vital Signs

Vital signs to be collected throughout the course of the study include systolic and diastolic blood pressures (mmHg), heart rate (bpm), body temperature (° C./° F.), and weight (kg). Vital signs will be summarised using univariate statistics (N, arithmetic average, standard deviation, median, and range) for each clinical assessment and presented for the cohort of patients who have data at the initial baseline visit and at least one the specific follow-up visits. In addition to the univariate statistics, the changes from baseline to each follow-up assessment visit will be analysed using a paired-difference t-test for the within-group mean change from baseline. Additionally, 95% confidence interval limits for the mean change from baseline will also be reported.

The incidence rates of clinically notable vital sign changes, including the criteria for clinically notable, will be summarized and presented in a Patient Data Listing. Vital signs and body weight abnormalities of potential clinical significance will be defined as follows:
Systolic blood pressure change 20 mmHg and a systolic blood pressure value that was ≧180 or ≦90 mmHg
Diastolic blood pressure change ≧15 mmHg and a diastolic blood pressure value that was ≧105 or ≦50 mmHg
Pulse change of ≧15 bpm and a pulse value that was ≧120 or ≦50 bpm
Temperature change of 1° C./2° F. and a temperature value that was ≧38° C./101° F.
Body weight decrease 5%
Clinically significant abnormal vital signs will be flagged and presented using counts by study visit.

An additional listing will be provided for those patients who have clinically significant vital sign abnormalities.

Other Safety Parameters

All other safety parameters will be listed by patient, treatment assignment, and study treatment period.

Laboratory Parameters

Haematology, biochemistry and other laboratory data will be listed at each time point by treatment group and, for appropriate values, will be flagged using the signed laboratory ranges as High/Low/Within laboratory normal range (H, L).

Changes from baseline will also be listed and abnormal changes from baseline will be flagged.

An additional listing will be provided for those patients who have laboratory values that are abnormal and considered to be clinically significant.

Withdrawals

The number (%) of patients who withdraw from the study over time, along with their reasons for withdrawal, will be tabulated.

Deaths

All deaths occurring during the treatment period of study and its follow up period will be listed.

Determination of Treatment Group Comparability

Patient demographics and disease histories will be summarised for each treatment group and compared between treatment groups.

Treatment Assignment

Patients will be randomised using a stratified central randomisation scheme. Given the initial target enrolment and the proposed number of clinical sites, attempting to balance the enrolment on an intra-centre basis was not considered feasible using a deterministic randomisation scheme. For example, if patients were to be randomised intra-centre using randomised blocks of 4, and 50% of the sites failed to fill a complete block, an enrolment imbalance could develop between the 2 groups resulting in a loss of statistical power. To eliminate this potential imbalance, a central randomisation scheme will be used, balancing on blocks of 4 within geographical areas (usually countries) involving multiple sites.

Stratification

Patients will be stratified by selected standard of care, prognostic indicator (Motzer score), geographical area, and institution. The stratification will be performed by IVRS.

Following the announced ending of the trial, immunological and clinical response data were un-blinded. An exploratory analysis was undertaken with the primary aim of identifying potential correlates between parameters and enhanced patient survival.

### Example 3

### Details of Phase II Survival Analysis (CRC) Patients—Effect of factors on Patient Survival

The results of the trials described in the following papers were analysed: Vaccination of colorectal cancer patients with modified vaccinia Ankara delivering the tumour antigen 5T4 (TroVax) induces immune responses which correlate with disease control: a phase I/II trial. Harrop R, Connolly N, Redchenko I, Valle J, Saunders M, Ryan M G, Myers K A, Drury N, Kingsman S M, Hawkins R E, Carroll M W. Clin Cancer Res. 2006 Jun. 1; 12(11 Pt 1):3416-24.

An MVA-based vaccine targeting the oncofetal antigen 5T4 in patients undergoing surgical resection of colorectal cancer liver metastases. Elkord E, Dangoor A, Drury N L, Harrop R, Burt D J, Drijfhout J W, Hamer C, Andrews D, Naylor S, Sherlock D, Hawkins R E, Stern P L. J Immunother. 2008 November-December; 31(9):820-9.

Vaccination of colorectal cancer patients with TroVax given alongside chemotherapy (5-fluorouracil, leukovorin and irinotecan) is safe and induces potent immune responses. Harrop R, Drury N, Shingler W, Chikoti P, Redchenko I, Carroll M W, Kingsman S M, Naylor S, Griffiths R, Steven N, Hawkins R E. Cancer Immunol Immunother. 2008 July; 57(7):977-86.

Vaccination of colorectal cancer patients with modified vaccinia ankara encoding the tumour antigen 5T4 (TroVax) given alongside chemotherapy induces potent immune responses. Harrop R, Drury N, Shingler W, Chikoti P, Redchenko I, Carroll M W, Kingsman S M, Naylor S, Melcher A, Nicholls J, Wassan H, Habib N, Anthoney A. Clin Cancer Res. 2007 Aug. 1; 13(15 Pt 1):4487-94.

An exploratory analysis was undertaken with the primary aim of identifying potential correlates between parameters and enhanced patient survival.

Two RRC trials, namely:
Clinical trial phase II (single centre study) A prelimiary study of the safety, immunogencity and clinical efficacy of TroVax® given in conjunction with interleukin 2 (IL-2) in the treatment of stage IV renal cell cancer, IND Number 11433; and
A phase I/II feasibility trial to assess the safety, immunological activity and efficacy of TroVax® plus interferon α (IFN-α) in patients with advanced or metastatic renal cell cancer. EudraCT Number: 2006-000753-22. GTAC Number: 117.

Details of these trials can be found on www.clinicaltrials.gov.

### Example 4

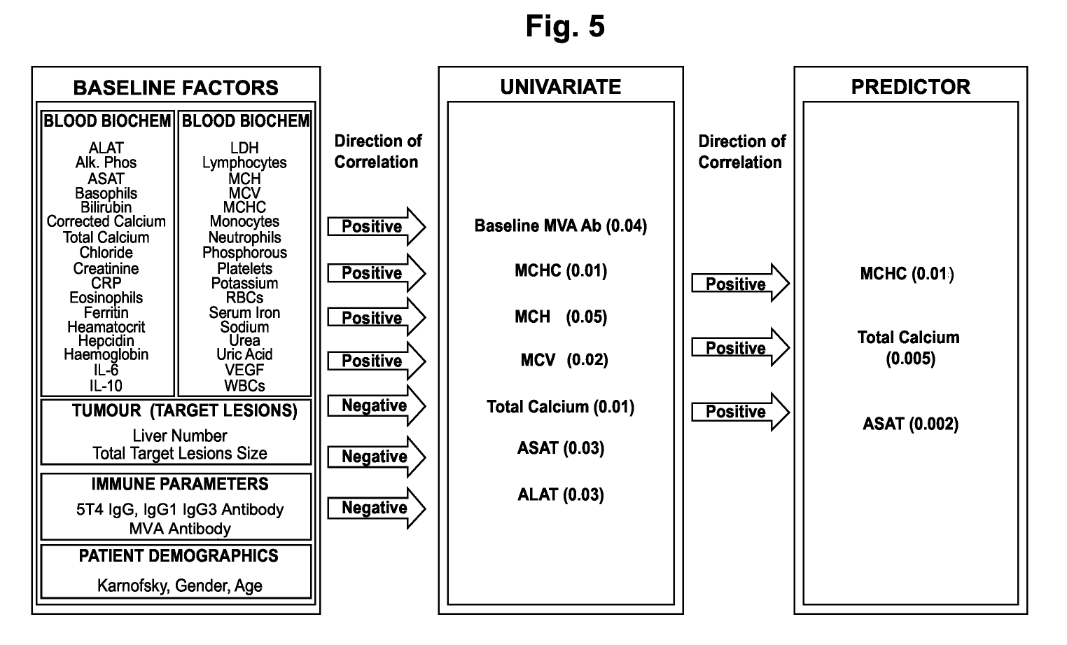

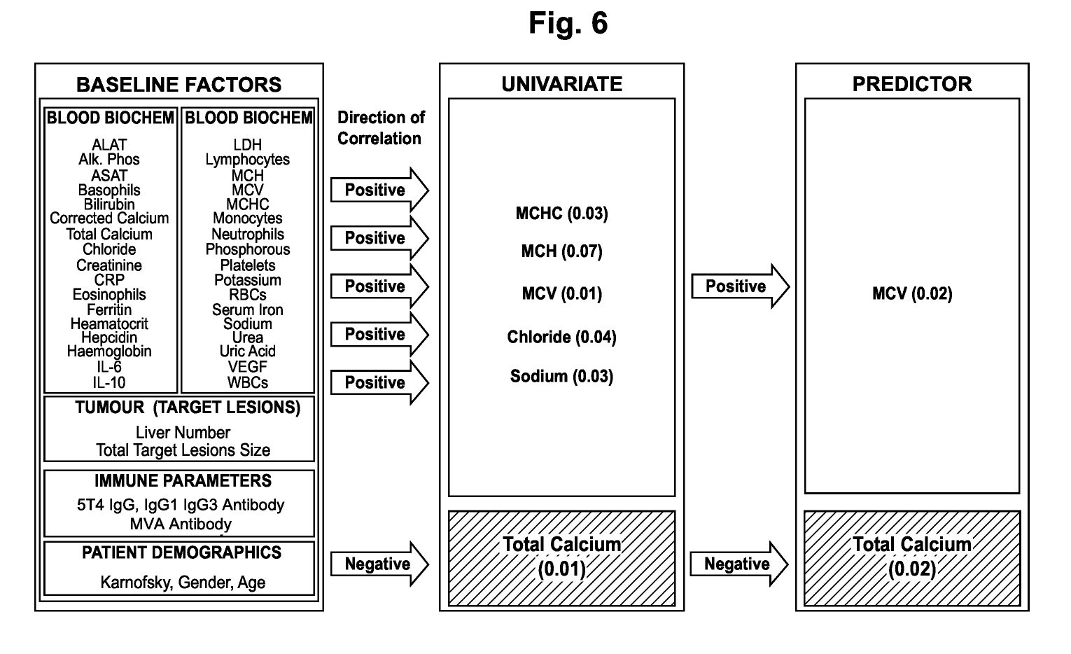

The association of a factor with treatment benefit was ascertained by fitting models of the effect on survival in the active and placebo groups in the TRIST study and evaluating whether or not the effect of the factor on survival differs between the two groups. The results are illustrated in FIGS. 5 and 6.

Various other preferred features and embodiments of the present invention will now be described with reference to the following numbered paragraphs (paras.):
1. A method for determining a prognosis for benefit for a cancer patient receiving immunotherapy treatment involving (a) measuring a baseline level of an antibody to a tumour associated antigen, haemoglobin and haematocrit in a sample from the cancer patient, and (b) comparing the baseline levels of the tumour associated antibody, haemoglobin and haematocrit in the sample to respective reference levels of tumour associated antibody, haemoglobin and haematocrit, wherein a lower baseline level of tumour associated antigen antibody, and a higher level of haemoglobin and a lower level of haematocrit in the sample correlates with increased benefit to the patient from immunotherapy treatment.
2. A method for determining a prognosis for benefit for a cancer patient receiving immunotherapy treatment involving (a) measuring a baseline level of an antibody to a tumour associated antigen, and at least one factor selected from the group consisting of: iron, ferritin, transferrin saturation, transferrin receptor, mean corpuscular volume, mean corpuscular haemoglobin concentration, zinc protoporphyrin, reticulocyte haemoglobin, bone marrow iron and red blood cells in a sample from the cancer patient immunotherapy treatment, and (b) comparing the levels of tumour associated antigen antibody, and said at least one factor to respective reference levels, wherein a lower level of tumour associated antibody, and a higher level of iron, ferritin, transferrin saturation mean corpuscular volume, mean corpuscular haemoglobin concentration, reticulocyte haemoglobin, bone marrow iron or red blood cells or a lower level of transferrin receptor or zinc protoporphyrin in the sample correlates with increased benefit to the patient from immunotherapy treatment.
3. A method for determining a prognosis for benefit for a cancer patient receiving immunotherapy treatment involving measuring the following factor in a sample from the cancer patient:

$$Factor = - 0.292 \times ( natural log of baseline level of an antibody to a tumour associated antigen ) + 0.0224 \times ( haemoglobin level in g \text{/} L ) + - 6.16 \times ( haematocrit as a fraction )$$

wherein a higher factor correlates with increased benefit to the patient from immunotherapy treatment.
4. A method for determining a prognosis for benefit for a cancer patient receiving immunotherapy treatment involving (a) measuring a factor as defined in para. 3 in a sample from the cancer patient, and (b) classifying the patient as belonging to either a first or second group of patients, wherein the first group of patients having a higher level of the factor is classified as having an increased likelihood of benefit than the second group of patients having a lower level of the factor.
4. The method of any one of paras. 1 to 4 wherein the method is for determining a prognosis for benefit for a cancer patient prior to receiving immunotherapy.
5. The method of any preceding para. wherein the measurement takes place prior to immunotherapy treatment.
6. The method of any preceding para. wherein a measurement or measurements is taken from one or more samples.
7. A method of predicting the responsiveness of a patient or patient population with cancer to treatment with immunotherapy, or for selecting patients or patient populations that will respond to immunotherapy, comprising comparing the differential levels of the factor as defined in para. 3.
8. A method according to any preceding para. wherein the tumour associated antibody is selected from the group consisting of: 5T4, HOM-MEL-40, HOM-MEL-55, HOM-MD-21, NY-COL-2, HOM-MD-397, HOM-NSCLC-22, HOM-MEL-2.4, MELAN-A/MART-1, MAGE-A1, MAGE-A2, MAGE-A3, MAGE-A4, MAGE-A6, MAGE-A8, MAGE-A10, MAGE-Al2, MAGE-B1-MAGE-B24, MAGE-C1/CT7, MAGE-CT10, GAGE-1, GAGE-8, PAGE-1, PAGE-4, XAGE-1, XAGE-3, LAGE-1a(1S), LAGE-1b(1L), NY-ESO-1, SSX-1-SSX-5, BAGE, SCP-1, TRP-2, CEA, PSA, MUC-1, tyrosinase, Her 2, survivin, and TERT.
9. The method according to any preceding para. wherein the cancer is invasive carcinoma of the Ampulla of Vater, breast, colon, endometrium, pancreas, or stomach; a squamous carcinoma of the bladder, cervix, lung or oesophagus; a tubulovillous adenoma of the colon; a malignant mixed Mullerian tumour of the endometrium; a clear cell carcinoma of the kidney; a lung cancer (large cell undifferentiated, giant cell carcinoma, broncho-alveolar carcinoma, metastatic leiomyosarcoma); an ovarian cancer (a Brenner tumour, cystadenocarcinoma, solid teratoma); a cancer of the testis (seminoma, mature cystic teratoma); a soft tissue fibrosarcoma; a teratoma (anaplastic germ cell tumours); or a trophoblast cancer (choriocarcimoma (e.g. in uterus, lung or brain), tumour of placental site, hydatidiform mole).
10. The method according to para. 9 wherein the cancer is renal, prostate, breast, ovarian or colorectal cancer.
11. The method according to any preceding para. wherein the immunotherapy comprises use of a poxvirus vector.
12. The method according to any preceding para. wherein the immunotherapy comprises use of 5T4 tumour associated antigen.
13. The method according to any one of paras. 1 to 4 or paras. referring thereto wherein the baseline level of antibody to a tumour associated antigen is the baseline level of 5T4 antibody.
14. The method according to any preceding para. wherein the immunotherapy comprises use of a Modified Vaccinia Ankara viral vector expressing the 5T4 tumour associated antigen.

### REFERENCES

1. Jemal A, Siegel R, Ward E, Murray T, Xu J, Thun M J. Cancer statistics, 2007. CA Cancer J Clin. 2007; 57:43-66.
2. Costa L J, Drabkin H A. Renal cell carcinoma: new developments in molecular biology and potential for targeted therapies. Oncologist. 2007; 12:1404-15.
3. Hole N, Stern P L. A 72 kD trophoblast glycoprotein defined by a monoclonal antibody. Br J Cancer. 1988; 57:239-46.
4. Southall P J, Boxer G M, Bagshawe K D, Hole N, Bromley M, Stern P L. Immunohistological distribution of 5T4 antigen in normal and malignant tissues. Br J Cancer. 1990; 61:89-95.
5. Starzynska T, Marsh P J, Schofield P F, Roberts S A, Myers K A, Stern P L. Prognostic significance of 5T4 oncofetal antigen expression in colorectal carcinoma. Br J Cancer. 1994; 69:899-902.
6. Harrop R, Shingler W, Kelleher M, De Belin J, Treasure P. Cross-Trial Analysis of Immunological and Clinical Data Resulting from Phase I and II Trials of MVA-5T4 (TroVax®) in Colorectal, Renal and Prostate Cancer Patients. J Immunother. 2010:accepted for publication.
7. Amato R J, Drury N, Naylor S, Jac J, Saxena S, Cao A, et al. Vaccination of Prostate Cancer Patients With Modified Vaccinia Ankara Delivering the Tumour Antigen 5T4 (TroVax): A Phase 2 Trial. J Immunother. 2008.
8. Amato R J, Shingler W, Goonewardena M, de Belin J, Naylor S, Jac J, et al. Vaccination of renal cell cancer patients with modified vaccinia Ankara delivering the tumour antigen 5T4 (TroVax) alone or administered in combination with interferon-alpha (IFN-alpha): a phase 2 trial. J Immunother. 2009; 32:765-72.
9. Amato R J, Shingler W, Naylor S, Jac J, Willis J, Saxena S, et al. Vaccination of renal cell cancer patients with modified vaccinia ankara delivering tumour antigen 5T4 (TroVax) administered with interleukin 2: a phase II trial. Clin Cancer Res. 2008; 14:7504-10.
10. Elkord E, Dangoor A, Drury N L, Harrop R, Burt D J, Drijfhout J W, et al. An MVA-based vaccine targeting the oncofetal antigen 5T4 in patients undergoing surgical resection of colorectal cancer liver metastases. J Immunother. 2008; 31:820-9.
11. Harrop R, Connolly N, Redchenko I, Valle J, Saunders M, Ryan M G, et al. Vaccination of colorectal cancer patients with modified vaccinia Ankara delivering the tumour antigen 5T4 (TroVax) induces immune responses which correlate with disease control: a phase I/II trial. Clin Cancer Res. 2006; 12:3416-24.
12. Harrop R, Drury N, Shingler W, Chikoti P, Redchenko I, Carroll M W, et al. Vaccination of colorectal cancer patients with modified vaccinia ankara encoding the tumour antigen 5T4 (TroVax) given alongside chemotherapy induces potent immune responses. Clin Cancer Res. 2007; 13:4487-94.
13. Kaufman H L, Taback B, Sherman W, Kim D W, Shingler W H, Moroziewicz D, et al. Phase II trial of Modified Vaccinia Ankara (MVA) virus expressing 5T4 and high dose Interleukin-2 (IL-2) in patients with metastatic renal cell carcinoma. J Transl Med. 2009; 7:2.
14. Harrop R, Drury N, Shingler W, Chikoti P, Redchenko I, Carroll M W, et al. Vaccination of colorectal cancer patients with TroVax given alongside chemotherapy (5-fluorouracil, leukovorin and irinotecan) is safe and induces potent immune responses. Cancer Immunol Immunother. 2007.
15. Hawkins R E, Macdermott C, Shablak A, Hamer C, Thistlethwaite F, Drury N L, et al. Vaccination of patients with metastatic renal cancer with modified vaccinia Ankara encoding the tumour antigen 5T4 (TroVax) given alongside interferon-alpha. J Immunother. 2009; 32:424-9.
16. Dallman P R. Iron deficiency and the immune response. Am J Clin Nutr. 1987; 46:329-34.
17. Ekiz C, Agaoglu L, Karakas Z, Gurel N, Yalcin I. The effect of iron deficiency anaemia on the function of the immune system. Hematol J. 2005; 5:579-83.
18. Kramer J L, Baltathakis I, Alcantara O S, Boldt D H. Differentiation of functional dendritic cells and macrophages from human peripheral blood monocyte precursors is dependent on expression of p21 (WAF1/CIP1) and requires iron. Br J Haematol. 2002; 117:727-34.
19. Zhu J, Martinez J, Huang X, Yang Y. Innate immunity against vaccinia virus is mediated by TLR2 and requires TLR-independent production of IFN-beta. Blood. 2007; 109:619-25.
20. Hutchens M A, Luker K E, Sonstein J, Nunez G, Curtis J L, Luker G D. Protective effect of Toll-like receptor 4 in pulmonary vaccinia infection. PLoS Pathog. 2008; 4:e1000153.
21. Lin T, Kwak Y H, Sammy F, He P, Thundivalappil S, Sun G, et al. Synergistic Inflammation Is Induced by Blood Degradation Products with Microbial Toll-Like Receptor Agonists and Is Blocked by Hemopexin. J Infect Dis. 2010; 202:624-32.
22. Amato, R., Hawkins, R., Kaufman, H. L., Thompson, J. A., Tomczak, P., Szczylik, C., McDonald, M., Eastty, S., Shingler, W., de Belin, J., Goonewardena, M., Naylor, S., and Harrop, R. “Vaccination of Metastatic Renal Cancer Patients with MVA-5T4: A Randomized, Double Blind, Placebo Controlled Phase III Study” Clin Cancer Res. 2010; 16(22): 5539-47.

1. A method for determining a prognosis for benefit for a cancer patient receiving immunotherapy treatment comprising
(a) measuring a level of haemoglobin and haematocrit in a sample from the cancer patient, and determining prognosis for the cancer patient benefiting from the immunotherapy by comparing the levels of haemoglobin and haematocrit in the sample to respective reference levels of haemoglobin and haematocrit, wherein a higher level of haemoglobin and a lower level of haematocrit in the sample correlates with increased benefit to the patient from immunotherapy treatment; or
(b) measuring a level of mean corpuscular haemoglobin concentration (MCHC) in a sample from the cancer patient, and determining prognosis for the cancer patient benefiting from the immunotherapy by comparing the level of MCHC in the sample to a reference level of MCHC, wherein a higher level of MCHC in the sample correlates with increased benefit to the patient from immunotherapy treatment; or
(c) measuring a level of mean corpuscular volume (MCV) in a sample from the cancer patient, and determining prognosis for the cancer patient benefiting from the immunotherapy by comparing the level of MCV in the sample to a reference level of MCV, wherein a higher level of MCV in the sample correlates with increased benefit to the patient from immunotherapy treatment; or
(d) measuring a level of mean cell haemoglobin (MCH) in a sample from the cancer patient, and determining prognosis for the cancer patient benefiting from the immunotherapy by comparing the level of MCH in the sample to a reference level of MCH, wherein a higher level of MCH in the sample correlates with increased benefit to the patient from immunotherapy treatment.

2. (canceled)

3. The method according to claim 1 said method further comprising measuring a level of red blood cell number in a sample from a cancer patient, and determining prognosis for the cancer patient benefiting from the immunotherapy by comparing the level of red blood cell number in a sample to a reference level of red blood cell number, wherein a high level of red blood cell number correlates with increased benefit to the patient from immunotherapy treatment

4-5. (canceled)

6. The method according to claim 1 said method further comprising measuring a level of total calcium in a sample from the cancer patient, and determining prognosis for the cancer patient benefiting from the immunotherapy by comparing the level of total calcium in the sample to a reference level of total calcium, wherein a lower level of total calcium in the sample correlates with increased benefit to the patient from immunotherapy treatment.

7. The method according to claim 1 said method further comprising measuring a level of aspartamine transaminase (ASAT) in a sample from the cancer patient, and determining prognosis for the cancer patient benefiting from the immunotherapy by comparing the level of ASAT in the sample to a reference level of ASAT, wherein a lower level of ASAT in the sample correlates with increased benefit to the patient from immunotherapy treatment.

8. The method according to claim 1 said method further comprising measuring a level of chloride in a sample from the cancer patient, and determining prognosis for the cancer patient benefiting from the immunotherapy by comparing the level of chloride in the sample to a reference level of chloride, wherein a higher level of chloride in the sample correlates with increased benefit to the patient from immunotherapy treatment.

9. The method according to claim 1 said method further comprising measuring a level of sodium in a sample from the cancer patient, and determining prognosis for the cancer patient benefiting from the immunotherapy by comparing the level of sodium in the sample to a reference level of sodium, wherein a higher level of sodium in the sample correlates with increased benefit to the patient from immunotherapy treatment.

10. The method according to claim 1 said method further comprising measuring a level of alanine transaminase (ALAT) in a sample from the cancer patient, and determining prognosis for the cancer patient benefiting from the immunotherapy by comparing the level of ALAT in the sample to a reference level of ALAT, wherein a lower level of ALAT in the sample correlates with increased benefit to the patient from immunotherapy treatment.

11. The method according to claim 1 said method further comprising measuring a baseline level of an antibody to a tumor associated antigen in a sample from the cancer patient, and determining prognosis for the cancer patient benefiting from the immunotherapy by comparing the baseline level of the an antibody to a tumor associated antigen in the sample to a reference level of antibody to a tumor associated antigen, wherein a lower baseline level of antibody to a tumor associated antigen in the sample correlates with increased benefit to the patient from immunotherapy treatment.

12. The method according to claim 1 said method further comprising measuring a level of at least one factor selected from the group consisting of: iron, ferritin, transferrin saturation, soluble transferrin receptor, total iron binding capacity, transferrin, zinc protoporphyrin, reticulocyte haemoglobin, bone marrow iron, hepcidin, C-reactive protein, interleukin 6, interleukin 10, vascular endothelial growth factor, interleukin 1, tumour necrosis factor alpha, and interferon gamma in a sample from the cancer patient receiving immunotherapy treatment, and determining prognosis for the cancer patient benefiting from the immunotherapy by comparing the levels of said at least one factor to respective reference levels, wherein a higher level of iron, transferrin saturation, reticulocyte haemoglobin, or bone marrow iron or a lower level of ferritin, soluble transferrin receptor, zinc protoporphyrin, hepcidin, C-reactive protein, interleukin 6, interleukin 10, vascular endothelial growth factor, interleukin 1, tumour necrosis factor alpha, or interferon gamma or normal levels of total iron binding capacity or transferrin in the sample correlates with increased benefit to the patient from immunotherapy treatment.

13. A method for determining a prognosis for benefit for a cancer patient receiving immunotherapy treatment comprising measuring the following factor in a sample from the cancer patient:
$$Factor = - 0.292 \times ( natural log of baseline level of an antibody to a tumor associated antigen ) + 0.0224 \times ( haemoglobin level in g \text{/} L ) + - 6.16 \times ( haematocrit as a fraction )$$
wherein a higher factor correlates with increased benefit to the patient from immunotherapy treatment.

14. A method for determining a prognosis for benefit for a cancer patient receiving immunotherapy treatment comprising
(a) measuring a factor as defined in claim 13 in a sample from the cancer patient, and
(b) classifying the patient as belonging to either a first or second group of patients, wherein the first group of patients having a higher level of the factor is classified as having an increased likelihood of benefit than the second group of patients having a lower level of the factor.

15. The method of claim 1, wherein the measuring is performed prior to the cancer patient to receiving immunotherapy.

16. (canceled)

17. A method for selecting patients or patient populations that will respond to immunotherapy, comprising comparing the differential levels of the factors defined in claim 1.

18. The method according to claim 11 wherein the tumor associated antigen is selected from the group consisting of: 5T4, WT1, MUC1, LMP2, HPV E6 E7, EGFR vIII, Her-2/neu, Idiotype, MAGE A3, p53, NY-ESO-1, PSMA, GD2, CEA, MelanA.MART1, Ras mutant, gp100, Proteinase 3, bcr-abl, Tyrosinase, Survivin, PSA, hTERT, EphA2, PAP, ML-IAP, AFP, EpCAM, ERG (TMPRSS2 ETS) fusion, NA17, PAX3, ALK, Androgen receptor, Cyclin B1, Polysialic acid, MYCN, RhoC, TRP-2, GD3, Fucosyl GM1, Mesothelin, PSCA, MAGE A1, sLE, CYP1B1, PLAC1, GM3, BORIS, Tn, GloboH, ETV6-AML, NY-BR-1, RGS5, SART3, STn, Carbonic Anhydrase IX, PAX5, OY-TES1, Sperm protein 17, LCK, HMWMAA, AKAP-4, SSX2, XAGE1, B7H3, Legumain, Tie2, Page4, VEGFR2, MAD-CT-1, FAP, PDGFR-β, MAD-CT-2, and Fos-related antigen 1.

19. The method according to claim 1 wherein the cancer is bladder cancer, bone cancer, brain cancer, breast cancer, cervical cancer, colorectal cancer, eye cancer, gallbladder cancer, Hodgkin's lymphoma, Kaposi's sarcoma, renal cancer, cancer of the larynx, leukemia, liver cancer, lung cancer, melanoma, mesothelioma, multiple myeloma, nasopharyngeal cancer, non-Hodgkin lymphoma, cancer of the esophagous, oral cancer, ovarian cancer, pancreatic cancer, cancer of the penis, prostate cancer, cancer of the salivary glands, cancer of the small intestine, stomach cancer, cancer of the testis, thyroid cancer, uterine cancer, cancer of the vagina or cancer of the vulva.

20. The method according to claim 19 wherein the cancer is renal cancer, prostate cancer, breast cancer, ovarian cancer, colorectal cancer or mesothelioma.

21. The method according to claim 1, wherein the immunotherapy comprises administering a poxvirus vector to the patient.

22. The method according to claim 1, wherein the immunotherapy comprises administering a 5T4 tumour associated antigen to the patient.

23. The method according to claim 11 wherein the baseline level of antibody to a tumor associated antigen is the baseline level of 5T4 antibody.

24. The method according to claim 1 wherein the immunotherapy comprises administering to the patient a Modified Vaccinia Ankara viral vector expressing the 5T4 tumour associated antigen.

25. The method according to claim 24 wherein the the human 5T4 tumour associated antigen gene in the vector is under the regulatory control of a modified mH5 promoter.

26. The method of claim 1, further comprising administering immunotherapy to a patient determined to have a prognosis of benefitting from the immunotherapy.
